(19) United States
(12) Patent Application Publication
(10) Pub. No.: US 2026/0256717 A1
(43) Pub. Date: Sep. 3, 2026

(54) THERAPEUTIC COMBINATIONS OF DRUGS, COMPANION DIAGNOSTICS AND METHODS FOR DOSAGING AND USING THEM

(71) Applicant: Bayesian Biotech, LLC, Basking Ridge, NJ (US)

(72) Inventors: John MAKI, Mendham, NJ (US); Michael NELSON, Lake Geneva, WI (US); Nieko PUNT, Maastricht (NL)

(21) Appl. No.: 18/879,952

(22) PCT Filed: Jul. 13, 2023
(86) PCT No.: PCT/US2023/027662

§ 371 Date: Dec. 30, 2024

Related U.S. Application Data
(60) Provisional application No. 63/388,969, filed on Jul. 13, 2022.
(60) Provisional application No. 63/390,553, filed on Jul. 19, 2022.
(60) Provisional application No. 63/422,366, filed on Nov. 3, 2022.
(60) Provisional application No. 63/442,581, filed on Feb. 1, 2023.
(60) Provisional application No. 63/466,792, filed on May 16, 2023.

(51) Int. Cl. A61K 31/138; A61K 31/4245; A61K 31/439; A61K 31/44; A61K 31/58; A61P 9/12; A61P 21/00; A61P 25/08; A61P 35/00; G01N 33/94

(52) U.S. Cl. CPC A61K 31/138; A61K 31/4245; A61K 31/439; A61K 31/44; A61K 31/58; A61P 9/12; A61P 21/00; A61P 25/08; A61P 35/00; G01N 33/94; G01N 2800/52

(57) ABSTRACT

In alternative embodiments, provided are drugs and companion diagnostics, or therapeutic drug combinations and a companion diagnostic comprising a dried blood spot or a volumetric dried blood spot, or an at-home high-volume liquid blood, collection device, including products of manufacture and kits, and methods, for using them. In alternative embodiments, provided are novel methods for dosaging therapeutic drug compositions. In alternative embodiments, provided are artificial intelligence (AI)-enabled therapeutic drug compositions (bMEDs™), including bMEDs™ amifampridine for Lambert Eaton Myasthenic Syndrome (LEMS), bMEDs™ deflazacort to Duchenne Muscular Dystrophy (DMD), bMEDs™ propranolol for Infantile Hemangioma, bMEDs™ serabelisib for PIK3CA-Related Overgrowth Spectrum (PROS), bMEDs™ sirolimus for Kaposiform Hemangioendothelioma with Kasabach-Merritt Phenomenon (“KHE w/KMP”), bMED™ everolimus for TSC-associated epilepsy, TSC-associated SEGA, TSC-associated renal angiomyolipoma, breast cancer, pNET, renal cancer, or allograph organ rejection prophylaxis, and bMEDs™ for rare diseases as provided herein.

### TECHNICAL FIELD

This invention generally relates to pharmacology and medicine. In alternative embodiments, provided are drugs and companion diagnostics, or therapeutic drug combinations and a companion diagnostic comprising a dried blood spot or a volumetric dried blood spot, or an at-home high-volume liquid blood, collection device, optionally with a time stamp, including products of manufacture and kits, and methods, for using them. In alternative embodiments, provided are novel methods for dosaging therapeutic drug compositions. In alternative embodiments, provided are methods comprising use of rolling or moving averages of administered drug blood concentrations to optimize further drug dosage administration. In alternative embodiments, artificial intelligence (AI)-enabled therapeutic drug compositions are provided herein that comprise individualized doses of a Narrow Therapeutic Index (“NTI”) drugs and artificial intelligence (AI)-enabled companion diagnostics for individualized pharmacokinetics (PK)-guided dosing; thus, using these methods enables patients to benefit from NTI drugs with optimally safe and effective drug concentrations and without the risks associated with high PK variability.

### BACKGROUND

Sirolimus (or rapamycin) is an mTOR1 (mammalian target of rapamycin 1 or or mechanistic target of rapamycin complex 1) inhibitor is used as an immunosuppressive drug intended to prevent organ rejection in renal transplantation, in adult patients with lymphangioleiomyomatosis and in pediatric patients for the treatment of various rare diseases such as vascular anomalies. Because sirolimus has a narrow therapeutic index, high interpatient pharmacokinetic variability and non-optimized dosing can lead to death or severe morbidity, particularly in pediatric patients, new and more effective methods of determining safe and efficacious dosaging are needed.

### SUMMARY

In alternative embodiments, provided are a therapeutic drug or drugs (or a therapeutic combination of drugs) and a companion diagnostic combination,
wherein the companion diagnostic comprises a dried blood spot, a volumetric dried blood spot, a volumetric absorptive microsampling (VAMS), or an at-home high-volume liquid blood, collection device, optionally with a time stamp device operably connected to or associated with the dried blood spot, a volumetric dried blood spot, or an at-home high-volume liquid blood collection device,
and optionally the dried blood spot or volumetric dried blood spot collection device comprises a filter paper card for the collection of one or more blood samples (or blood drops, optionally the blood drop is generated by a user or patient using a lancet or a fingerstick), and optionally the filter paper comprises one, two or more designated spaces for a blood drop to be placed, and optionally each filter paper and/or each designated space has a unique bar code or QR code, and optionally the dried blood spot, the volumetric dried blood spot, or the at-home high-volume liquid blood collection device comprises one or a plurality of lancets or fingersticks (or fingerprick), and optionally the filter paper is stored and optionally mailed in a gas-impermeable container (optionally a zipper bag), optionally containing one or more desiccant sachets to protect the blood specimens on the filter paper from moisture contamination,
and optionally the volumetric dried blood spot, volumetric absorptive microsampling (VAMS) or the at-home high-volume liquid blood collection device comprises a hand-held device comprising a plurality of microneedles or volumetric needles, wherein the plurality of microneedles or volumetric needle are capable of capturing a predefined volummetric blood sample, for example, a blood sample of between about 5 μl and 500 μl, or between about 10 μl and 100 μl, or about 5 μl, 10 μl, 15 μl, 20 μl, 25 μl, 30 μl or 35 μl, and optionally the volumetric dried blood spot, volumetric absorptive microsampling (VAMS) or the at-home high-volume liquid blood collection device comprises a MITRA™ device (by NEOTERYX™), or a bladeless microneedle array as in a TAPMICRO™ or HALO™ device (yourbio, Medford, MA),
and optionally the therapeutic drug or drugs and the companion diagnostic combination are packaged or contained in a package or container having a container or receptacle for each drug, optionally the therapeutic drug or drugs are packaged in a kit, a sachet, a blister pack, a packet, a lidded blister, a blister card, a clamshell, a tray or a shrink wrap,
and optionally the package or container, or the kit, sachet, blister pack, packet, lidded blister, blister card, clamshell, tray or shrink wrap further comprises a time stamp device for each drug container or receptacle, or a time stamp device operably connected to or associated with each drug container or receptacle,
and optionally the time stamp device records, transmits to a remote device, or displays to a user as a readable output data comprising when (the time) each of the separate containers is opened, and optionally data comprising how often each of the separate containers is opened, and optionally the time stamp device data is transmitted to the use, or a patient, a caregiver, a technician, a nurse and/or a physician, or optionally to a central database or to a remote computer or device,
and optionally each (separate) drug container or receptable comprises a unique Quick Response (QR) code, or unique two-dimensional matrix barcode, to identify each drug container or receptable and each drug or drugs contained in each drug container or receptable,
the therapeutic drug or drugs (or a therapeutic combination of drugs) comprising or having contained therein at least one drug or a combination of drugs, wherein the at least one drug or the combination of drugs is in a group selected from (and in alternative embodiments, for the phrase Narrow Therapeutic Index (“NTI”) drugs as used herein all of the following drugs are considered NTI drugs):
(a) a beta adrenergic blocker (or beta blocker), or a salt, hydrate, solvate, tautomer, stereoisomer or deuterated isoform thereof, wherein optionally the beta adrenergic blocker comprises propranolol (or INDERAL™), atenolol (or TENORMIN™), metoprolol (or LOPRESOR™), a carvedilol (or COREG™), a Bisoprolol (or ZEBETA™), nadolol (or CORGARD™), a nebivolol (or BISTOLIC™), timolol (or TIMOL™) or a combination thereof,
(b) a glucocorticoid, or a salt, hydrate, solvate, tautomer, stereoisomer or deuterated isoform thereof, wherein optionally the glucocorticoid comprises deflazacort (or EMFLAZA™, CALCORT™), vamorolone (or 17α,21-Dihydroxy-16α-methylpregna-1,4,9(11)-triene-3,20-dione), prednisone (or DELTASONE™ LIQUID PRED™, ORASONE™), prednisolone (or ORAPRED™, PEDIAPRED™ MILLIPRED™) or a combination thereof.
(c) an mTOR (mammalian target of rapamycin) inhibitor, or a salt, hydrate, solvate, tautomer, stereoisomer or deuterated isoform thereof, wherein optionally the mTOR inhibitor comprises everolimus (or AFINITOR™, ZORTRESS™), sirolimus (also known as rapamycin) (or RAPAMUNE™, FYARRO™) or a combination thereof,
and optionally in alternative embodiments, everolimus is formulated for oral administration, and optionally supplied on a blister card that time-stamps the use of the single dose tablet, capsule, or pre-filled syringe of everolimus solution or the blood collection device,
and optionally in alternative embodiments, everolimus is formulated in tablets or capsules or pre-filled syringe for administration, optionally to provide 0.5 mg, 0.6 mg, 0.7 mg, 0.85 mg, 1.05 mg, 1.25 mg, 1.5 mg, 1.8 mg, 2.1 mg, 2.5 mg, 3.0 mg, 3.6 mg, 4.3 mg, 5.0 mg, 6.0 mg, 7 mg, 8.5 mg, 10 mg, 12 mg, 15 mg, 18 mg, 21 mg, 25 mg, 30 mg, 35 mg or 40 mg of everolimus;
(d) a phosphatidylinositol-4,5-bisphosphate 3-kinase, catalytic subunit alpha inhibitor (or PIK3Ca inhibitor, or p110α protein), or a salt, hydrate, solvate, tautomer, stereoisomer or deuterated isoform thereof, wherein optionally the PIK3Ca inhibitor comprise alpelisib (or PIQRAY™, VIJOICE™), inavolisib (or GDC-0077 (Genentech)), serabelisib (or [6-(2-amino-1,3-benzoxazol-5-yl)imidazo[1,2-a]pyridin-3-yl]-morpholin-4-ylmethanone), CYH-33, MN1611 or a combination thereof; and
(e) a voltage-gated potassium channel blocker, or a salt, hydrate, solvate, tautomer, stereoisomer or deuterated isoform thereof, wherein optionally the voltage-gated potassium channel blocker comprises amifampridine (or for example FIRDAPSE™, RUZURGI™), dalfampridine (or 4-aminopyridine, or pyridin-4-amine);
(f) a mineralocorticoid receptor antagonist, or a salt, hydrate, solvate, tautomer, stereoisomer or deuterated isoform thereof, wherein optionally the mineralocorticoid receptor antagonist comprises spironolactone (for example, ALDACTONE™, SPIRACTIN™, VEROSPIRON™) or eplerenone (for example, INSPRA™, EPNONE™, DOSTEREP™);
(g) a 4-phenylbutyric acid, or a salt, hydroate, solvate, tutomer, steroisomer or deuterated isoform thereof, wherein optional the 4-phenylbutyric acid is a glycerol phenylbutyrate (for example, RAVICTI™), sodium phenylbutyrate (for example, BUPHENYL™);
(h) a protein kinase inhibitor, or a salt, hydrate, solvate, tautomer, stereoisomer or deuterated isoform thereof, wherein optionally the protein kinase inhibitor is an abemaciclib (or VERZENIO™, VERZENIOS™), an acalabrutinib (or CALQUENCE™), an afatinib, an alectinib, a avapritinib, an axitinib, a binimetinib, a baricitinib, a binimetinib, a bosutinib, a brigatinib, a cabozantinib, a ceritinib, a capmatinib, a cobimetinib, a copanlisib, a crizotinib, a dabrafenib, a dacomitinib, a dasatinib, a duvelisib, an erdafinib, an encorafenib, an erlotinib, a fasudil, fedratinib, a filgotinib, a fostamatinib, a gefitinib, a gilteritinib, an ibrutinib, am idelalisib, an imatinib, an infigratinib, a lapatinib, a larotrectinib, a lenvatinib, a lestaurtinib, a lorlatinib, a masitinib, a midostaurin, a momelotinib, a neratinib, a netarsudil, a nilotinib, a nintedanib, an oclacitinib, an osimertinib, a pacritinib, a palbociclib, a pazopanib, a peficitinib, a pexidartinb, a ponatinib, a regorafenib, a ribociclib, a ruxolitinib, a selumetinib, a sorafenib, a sunitinib, a tepotinib, a tivozanib, a tofacitinib, a trametinib, a tucatinib, a upadacitinib, a vandetanib, a vemurafenib, a zanubrutinib, or any combination thereof;
(i) an immunomodulatory drugs (iMiDs) or a salt, hydrate, solvate, tautomer, stereoisomer or deuterated isoform thereof, wherein optionally the immune-modulatory drug comprises: a lenalidomide, a pomalidomide, a thalidomide, or any combination thereof;
(j) a sphingosine-1-phosphate receptor modulator or a salt, hydrate, solvate, tautomer, stereoisomer or deuterated isoform thereof, wherein optionally the sphingosine-1-phosphate receptor modulators is a fingolimod, an ozanimod, a ponesimod, or any combination thereof;
(k) a siponimod, a calcineurin inhibitor, or a salt, hydrate, solvate, tautomer, stereoisomer or deuterated isoform thereof, wherein optionally the calcineurin inhibitor comprises tacrolimus, cyclosporin, or any combination thereof;
(l) an inosine-5′-monophosphate dehydrogenase inhibitor, or a salt, hydrate, solvate, tautomer, stereoisomer or deuterated isoform thereof, wherein optionally the inosine-5′-monophosphate dehydrogenase inhibitor comprises a mycophenolate mofetile;
(m) cyclooxygenase-2 selective non-steroidal anti-inflammatory drugs or a salt, hydrate, solvate, tautomer, stereoisomer or deuterated isoform thereof, wherein optionally the cyclooxygenase-2 selective non-steroidal anti-inflammatory drugs is a celecoxib, an etodolac, an etoricoxib, a meloxicam, a rofecoxib, a valdecoxib, or any combination thereof,
(n) lithium drug or a salt, hydrate, solvate, tautomer, stereoisomer or a deuterated isoform thereof, wherein optionally the lithium salt comprises a lithium carbonate, lithium acetate, lithium sulfate, lithium citrate, lithium orotate, lithium gluconate or any combination thereof,
(o) an angiotensin converting enzyme inhibitor, an enalapril (or VASCOTECH™), a lisinopril (or PRINIVIL™ or ZESTRIL™), a perindopril (or ACEON™)
(p) an angiotensin receptor blocker, a losartan (or COZAAR™)
(q) an antiepileptic drug, alprazolam (or XANAX™), cannabidiol (or EPIDIOLEX™), carbamazepine (or TEGRETOL™, CARBATROL™), clobazam (or FRISIUM™, ONFI™), Clonazepam (or KLONOPIN™, RIVOTRIL™), diazepam (or VALIUM™), ethosuximide (or ZARONTIN™), fenfluramine (or FINTEPLA™), Flurpirtine (or KATADOLON™), gabapentin (or NEURONTIN™ GRALISE™), Ganaxolone (or GANAXONE™, ZTALMY™), Lacosamide (or VIMIPAT™), Lorazepam (or ATIVAN™), Levetiracetam (or KEPPRA™ ROWEEPRA™), Lamotrigine (or LAMICTAL™), Memantine (or NAMENDA™) Methsuximide (or CELONTIN™), Oxcarbazepine (or TRILEPTAL™, OXTILAR XR™), Perampanel (or FYCOMPA™), Pyridoxine (or vitamin B6), Phenobarbital (or LUMINAL™), Phenytoin (or DILANTIN™, EPTOIN™), Retigabine (or POTIGA™, TROBALT™), Rufinamide (or BANZEL™, INOVELON™), Stiripentol (or DIACOMIT™), Topiramate (or TOPAMAX™, TROKENDI XR™), Valproic Acid (or DEPAKENE™, DEPAKOTE™), Vigabatrin (or SABRIL™), Vinpocetine (or CAVINTON™, INTELECTOL™), Zonisamide (or ZONEGRAN™) or any combination thereof,
(r) an oxadiazole capable of making a ribosome less sensitive to premature stop codons (an effect referred to as “read-through”) by promoting insertion of near-cognate tRNA at nonsense codons, or ataluren or 3-[5-(2-Fluorophenyl)-1,2,4-oxadiazol-3-yl]benzoic acid, or TRANSLARNA™, and/or
(s) a colchicine (or COLCRYS™, GL OPERBA™, MITIGARE™).

In alternative embodiments the therapeutic combination of drugs comprises a combination of a drug or drugs as set forth in:
(c) and (r), or everolimus or sirolimus (also known as rapamycin) and ataluren;
(c) and (a), or everolimus or sirolimus and a beta adrenergic blocker (or beta blocker) or propranolol;
(c) and (b), or everolimus or sirolimus and a glucocorticoid, or deflazacort;
(c) and (d), or everolimus or sirolimus and a phosphatidylinositol-4,5-bisphosphate 3-kinase, catalytic subunit alpha inhibitor or alpelisib, inavolisib or serabelisib;
(c) and (e), or everolimus or sirolimus and a voltage-gated potassium channel blocker, or amifampridine or dalfampridine;
(c) and (f), or everolimus or sirolimus and a mineralocorticoid receptor antagonist, or spironolactone or eplerenone;
(c) and (g), or everolimus or sirolimus and a 4-phenylbutyric acid, or glycerol phenylbutyrate or sodium phenylbutyrate;
(c) and (h), or everolimus or sirolimus and a protein kinase inhibitor, or abemaciclib;
(c) and (i), or everolimus or sirolimus and at least one immunomodulatory drug (iMiDs), or lenalidomide, pomalidomide or thalidomide;
(c) and (j), or everolimus or sirolimus and a sphingosine-1-phosphate receptor modulator, or fingolimod, ozanimod or ponesimod;
(c) and (k), or everolimus or sirolimus and siponimod, a calcineurin inhibitor, tacrolimus or cyclosporin;
(c) and (l), or everolimus or sirolimus and an inosine-5′-monophosphate dehydrogenase inhibitor, or mycophenolate mofetile;
(c) and (m), or everolimus or sirolimus and a cyclooxygenase-2 selective non-steroidal anti-inflammatory drug, or celecoxib, etodolac, etoricoxib, meloxicam, rofecoxib or valdecoxib;
(c) and (n), or everolimus or sirolimus and a lithium drug;
(c) and (o), or everolimus or sirolimus and an angiotensin converting enzyme inhibitor, or enalapril, lisinopril or perindopril;
(c) and (p), or everolimus or sirolimus and an angiotensin receptor blocker or losartan;
(c) and (q), or everolimus or sirolimus and an antiepileptic drug, or cannabidiol, or ganaxolone, or valproic acid, or vigabatrin, or diazepam or gabapentin;
(c) and (s), or everolimus or sirolimus and a colchicine;
(r) and (b), or ataluren and a glucocorticoid, or deflazacort;
(r) and (a), or ataluren and a beta adrenergic blocker (or beta blocker) or propranolol;
(r) and (d), or ataluren and a phosphatidylinositol-4,5-bisphosphate 3-kinase, catalytic subunit alpha inhibitor or alpelisib, inavolisib or serabelisib;
(r) and (e), or ataluren and a voltage-gated potassium channel blocker, or amifampridine or dalfampridine;
(r) and (f), or ataluren and a mineralocorticoid receptor antagonist, or spironolactone or eplerenone;
(r) and (g), or ataluren and a 4-phenylbutyric acid, or glycerol phenylbutyrate or sodium phenylbutyrate;
(r) and (h), or ataluren and a protein kinase inhibitor, or abemaciclib;
(r) and (i), or ataluren and an immunomodulatory drug (iMiDs), or lenalidomide, pomalidomide or thalidomide;
(r) and (j), or ataluren and a sphingosine-1-phosphate receptor modulator, or fingolimod, ozanimod or ponesimod;
(r) and (k), or ataluren and siponimod, a calcineurin inhibitor, tacrolimus or cyclosporin;
(r) and (l), or ataluren and an inosine-5′-monophosphate dehydrogenase inhibitor, or mycophenolate mofetile;
(r) and (m), or ataluren and a cyclooxygenase-2 selective non-steroidal anti-inflammatory drug, or celecoxib, etodolac, etoricoxib, meloxicam, rofecoxib or valdecoxib;
(r) and (n), ataluren and a lithium drug;
(r) and (o), or ataluren and an angiotensin converting enzyme inhibitor, or enalapril, lisinopril or perindopril;
(r) and (p), or ataluren and an angiotensin receptor blocker or losartan;
(r) and (q), or ataluren and an antiepileptic drug, or diazepam or gabapentin; and/or
(r) and (s), or ataluren and colchicine.

In alternative embodiment of the drug or therapeutic combination of drugs and the companion diagnostic:
the therapeutic combination of drugs comprises at least two drugs comprising:
   (a) a beta adrenergic blocker and an mTOR inhibitor;
   (b) a glucocorticoid and an mTOR inhibitor;
   (c) a glucocorticoid and a voltage-gated potassium channel blocker;
   (d) an mTOR inhibitor and a PIK3Ca inhibitor; and/or
   (e) a glucocorticoid and a mineralocorticoid receptor antagonist;
the drug or the therapeutic combination of drugs further comprises:
   (a) casimersen (or AMONDYS 45™);
   (b) viltolarsen (or VIL,TEPSO™, or VYONSIA 53™)
   (c)
   PF-06939926™ (Pfizer),
   (d) SRP-9001™ (Sarepta Therapeutics, Roche),
   (e) SPR-5051™ (Sarepta Therapeutics),
   (f) DS-5141b™ (Daiichi-Sankyo); or
   (g) any combination of (a) to (f);
two or more of the drugs are formulated as separate compositions, or two or more of the drugs are formulated into one composition or drug formulation (two or more drugs are formulated together);
one or two or more of the drugs are packaged individually, or are packaged together, or packaged in any combination, in a single package, a plurality of packages or packettes, or a blister packet, a sachet, a lidded blister or blister card or packets, or a shrink wrap;
one or two or more or all of the drugs are formulated or manufactured as a parenteral formulation, an aqueous solution, a liposome, an injectable solution, a tablet, a sachet, a pill, a lozenge, a capsule, a caplet, a spray, a sachet, an inhalant, a powder, a freeze-dried powder, an inhalant, a patch, a gel, a geltab, a nanosuspension, a nanoparticle, a nanoliposome, a microgel, a pellet, a suppository or any combination thereof,
and optionally the drug delivery device or product of manufacture is or comprises an implant;
the one or two or more or all of the drugs are formulated or manufactured together in one parenteral formulation, one aqueous solution, one liposome, one injectable solution, one freeze-dried powder, one feed, one food, one food supplement, one pellet, one lozenge, one liquid, one elixir, one aerosol, one inhalant, one adhesive, one spray, one powder, one freeze-dried powder, one patch, one tablet, one pill, one capsule, one gel, one geltab, one lozenge, one caplet, one sachet, one nanosuspension, one nanoparticle, one nanoliposome, one microgel or one suppository;
the one or two or more or all of the drugs are packaged in dosages that match a chrono-dosing regimen to match an optimal dose for the time of day; and/or
the one or two or more or all of the drugs are formulated in a unit dosage amount ranging from between about 0.1 mg, 0.25 mg, 0.5 mg, 1 mg, 2.5 mg, 5 mg, 10 mg, 25 mg, 50 mg, or 100 mg to about 0.5 gram or 1 gram or 2 gram or 4 gram (gm), and optionally the one or two or more or all of the drugs are formulated as immediate release formulations or controlled release formulations.

In alternative embodiments, provided are methods for treating, ameliorating, preventing, slowing the advance of, or lessoning the symptoms of:
a vascular anomaly,
   and optionally the vascular anomaly comprises infantile hemangioma, Arteriovenous Malformation (AVM), Cerebral Cavernous Malformation (CCM), Hereditary Hemorrhagic Telangiectasia (HHT), Von Hippel-Lindau Disease (VHL), angiosarcoma, Kaposiform Hemangioendothelioma (KHE), Kaposiform Lymphangioendothelioma (KLA). Microcystic Lymphatic Malformations or a PIKCA-Related Overgrowth Spectrum (PROS),
   and optionally the PROS is: Fibro-adipose vascular anomaly Hemihyperplasia—multiple lipomatosis syndrome, CLOVES (congenital lipomatous (fatty) overgrowth, vascular malformations, epidermal nevi and scoliosis/skeletal/spinal anomalies) syndrome Macrodactyly, Facial infiltrating lipomatosis, Macrocephaly-capillary malformation, Dysplastic megalencephaly or Klippel-Trenaunay syndrome;
a Muscular Dystrophy (MD),
   and optionally the MD is Duchenne Muscular Dystrophy (DMD), Beckers Muscular Dystrophy, Congenital Muscular Dystrophy, Facioscapulohumeral Dystrophy, Limb-Girdle Muscular Dystrophy (LGMD), Myotonic Dystrophy Type 1, Myotonic Dystrophy Type 2, or Lambert Eaton Myasthenic Syndrome (LEMS);
a nervous system disorder,
   and optionally the nervous system disorder is Huntington's Disease, Tuberous Sclerosis Complex (TSC), or subependymal giant cell astrocytoma (SEGA) associated with TSC, or a developmental and Epileptic Encephalopathies, a Dravet Syndrome, a Epilepsy in Infancy with Migrating Focal Seizures. a Landau Kleffner Syndrome, a Lennox-Gastaut Syndrome (LGS), a Myoclonic Atonic Epilepsy (Doose Syndrome), a Rett Syndrome, a West Syndrome, or, Epilepsies Associated with Specific Genetic or Metabolic Disorders: a GLUT1 Deficiency Syndrome, a Neuronal Ceroid Lipofuscinosis (NCL), a Protocadherin 19 Female-Limited Epilepsy (PCDH19 Epilepsy), Pyridoxamine 5′-Phosphate Oxidase Deficiency, Trisomy 21 and Epileptic Spasms, TSC associated seizures or other rare or refractory epilepsies including Epilepsy with Continuous Spike-Wave during Sleep (CSWS): Febrile Infection-Related Epilepsy Syndrome (FIRES) and medically intractable epilepsies, a complex partial seizures, infantile spasms, transitional focal epilepsy in infancy, a status epilepticus, partial seizures, catamenial epilepsies, early myoclonic encephalopathy, persistent spike epilepsy in slow wave sleep (except Landau-Kleffner syndrome), hypothalamic epilepsy, dose syndrome (myoclonic-standing disability epilepsy), myoclonic states of non-progressive encephalopathy, tawnian syndrome or early epileptic encephalopathy in infancy, glycine encephalopathy, 15q repeat syndrome (Dup 15 q), epilepsy associated with CHD2, cyclin-dependent kinase-like 5 (CDKL 5), SCN1A, SCN2A, SCN8A, ARX, KCNA1, KCNA2, KCNT1, KCNQ2, HCN1, PCDH19, GRIN1, GRIN2A and GRIN2B mutations, a Smith-Magenis Syndrome, 1p36 deletion syndrome, 16p11.2 deletion syndrome, fragile X syndrome, trisomy 21, Angelman's Syndrome, Schaff-Yang Syndrome, Albright Hereditary Osteodystroph, Silver-Russell Syndrome, Alstrom Syndrome, Mental Retardation or WAGR or WAGRO Syndrome; Gillespie syndrome or cerebellar ataxia, Coffin-Lowry Syndrome, hypersomnia, excessive daytime sleepiness (EDS), excessive daytime sleepiness associated with central or obstructive sleep apnea, idiopathic hypersomnia, sleep related breathing disorders, obstructive sleep apnea, central sleep apnea, Sleep-Related Hypoventilation Disorders, Sleep-Related Hypoxemia disorder, Rapid eye movement (REM) sleep behavior disorder, restless leg syndrome, nightmare disorder, dream anxiety disorder, Alpers Disease, Alpers Syndrome, Kearns-Sayre syndrome (KSS). Leber hereditary optic neuropathy (LHON), Leigh disease, Leigh syndrome, Mitochondrial encephalopathy lactic acidosis and stroke-like episodes (MELAS), Myoclonic epilepsy and ragged-red fiber disease syndrome (MERRF), Pontocerebellar Hypoplasia Type 6 (PCH6), Autism or Autism Spectrum Disorder (ASD), Asperger spectrum, or a Pervasive Developmental Disorder (PDD), hypersomnia, excessive daytime sleepiness (EDS), excessive daytime sleepiness associated with central or obstructive sleep apnea, idiopathic hypersomnia, narcolepsy, narcolepsy type 1, sleep related breathing disorders, obstructive sleep apnea, central sleep apnea, Sleep-Related Hypoventilation Disorders, Sleep-Related Hypoxemia Disorder, Rapid Eye Movement (REM) sleep behavior disorder, restless leg syndrome (RLS), nightmare disorder, dream anxiety disorder or a chronic fatigue syndrome, Parkinson's Disease and Alzheimer's Disease;
a neuropsychiatric disorder,
   and optionally the neuropsychiatric disorder is TSC-Associated Neuropsychiatric Disorder (TAND), General Anxiety, Social Anxiety Disorder (Social Phobia), attention deficit hyperactivity disorder (ADHD) disruptive mood dysregulation disorder (DMDD), intermittent explosive disorder (IED), major depressive disorder (MDD), treatment resistant depression (TRD), obsessive-compulsive disorder (OCD), negative symptoms of schizophrenia, Parkinson's Disease, MDD Associated with Parkinson's Disease, oppositional defiant disorder (ODD);
a retinopathy,
   and optionally the retinopathy is a retinopathy of prematurity (ROP), Norrie disease retinopathy, Familial Exudative Vitreoretinopathy (FEVR), Coats' disease retinopathy or diabetic retinopathy;
a cardiomyopathy,
   and optionally the cardiomyopathy is infantile hypertrophic cardiomyopathy, infantile dilated cardiomyopathy, infantile restricted cardiomyopathy, Barth's syndrome, mitochondrial cardiomyopathy;
   a renal disease, and optionally, the renal disease is nephrotic syndrome;
a peripheral nervous system disorder,
   and optionally the peripheral nervous system disorder is Hereditary Neuropathy with liability to Pressure Palsies (HNPP), Neurofibromatosis Type 1 (NF1), Neurofibromatosis Type 2 (NF2), Schwannoma;
a neuroendocrine system disorder,
   and optionally the neuroendocrine system disorder is Pheochromocytomas (PHEO), paragangliomas (PGL), pheochromocytomas and paragangliomas (PPGL), Hereditary paraganglioma-pheochromocytoma (PGL/PCC),
allograft renal transplants, allograft liver transplants, allograft heart transplant;
a benign tumor, and wherein optionally the benign tumor is tuberous sclerosis complex (TSC)-associated subependymal giant cell astrocytoma (SEGA), cardiac rhabdomyoma, renal angiomyolipoma, PTEN hamartoma tumor syndrome (PHTS), Bannayan-Riley Ruvalcaba Syndrome (BRRS), Birt-Hogg-Dube Syndrome, Cowden's Syndrome, Proteus Syndrome or PTEN-related Proteus Syndrome;
a malignant neoplasm or cancer, wherein optionally the malignant neoplasm is a breast cancer, a hormone receptor positive breast tumor, a pancreatic neuroendocrine (pNET) or a renal cell carcinoma (RCC);
an autoimmune disorder, wherein optionally the autoimmune disorder is Crohn's Disease, Diabetes Type 1, Multiple Sclerosis, Myasthenia Gravis, Rheumatoid Arthritis, Lupus, Scleroderma and/or Psoriasis,
a dermatological disorder, for example, psoriasis, urticaria or angioedema;
a pulmonary disease, wherein optionally the pulmonary disease is a lymphangioleiomyomatosis (LAM); and/or is pulmonary arterial hypertension (PAH); and/or
a polyposis, wherein optionally the polyposis is familial adenomatous polyposis (FAP) or hereditary flat adenoma syndrome or a Juvenile polyposis syndrome (JPS),
comprising administering to an individual in need thereof a therapeutic amount of:
(a) a drug or a therapeutic drug combination and companion diagnostic as provided herein; or
(b) at least one drug, drug combination or formulation comprising:
   (1) a beta adrenergic blocker (or beta blocker), or a salt, hydrate, solvate, tautomer, stereoisomer or deuterated isoform thereof, wherein optionally the beta adrenergic blocker comprises propranolol (or INDERAL™), atenolol (or TENORMIN™), metoprolol (or LOPRESOR™), nadolol (or CORGARD™), timolol (or TIMOL™) or a combination thereof,
   (2) a glucocorticoid, or a salt, hydrate, solvate, tautomer, stereoisomer or deuterated isoform thereof, wherein optionally the glucocorticoid comprises deflazacort (or EMFLAZA™, CALCORT™) vamorolone (or 17α,21-Dihydroxy-16α-methylpregna-1,4,9(11)-triene-3,20-dione), prednisone (or DELTASONE™, LIQUID PRED™, ORASONE™), prednisolone (or ORAPRED™ PEDIAPRED™, MILLIPRED™) or a combination thereof.
   (3) an mTOR (mammalian target of rapamycin) inhibitor, or a salt, hydrate, solvate, tautomer, stereoisomer or deuterated isoform thereof, wherein optionally the mTOR inhibitor comprises everolimus (or AFINITOR™, ZORTRESS™), sirolimus (or rapamycin, or RAPAMUNE™, FYARRO™, ERAPA™, DRGT18-2) or a combination thereof,
   (4) a phosphatidylinositol-4,5-bisphosphate 3-kinase, catalytic subunit alpha inhibitor (or PIK3Ca inhibitor, or p110a protein), or a salt, hydrate, solvate, tautomer, stereoisomer or deuterated isoform thereof, wherein optionally the PIK3Ca inhibitor comprise alpelisib (or PIQRAY™, VIJOICE™), inavolisib (or GDC-0077 (Genentech)), serabelisib (or [6-(2-amino-1,3-benzoxazol-5-yl)imidazo[1,2-a]pyridin-3-yl]-morpholin-4-ylmethanone), CYH-33, MN1611 or a combination thereof,
   (5) a voltage-gated potassium channel blocker, or a salt, hydrate, solvate, tautomer, stereoisomer or deuterated isoform thereof, wherein optionally the voltage-gated potassium channel blocker comprises amifampridine (or FIRDAPSE™, RUZURGI™), dalfampridine (or 4-aminopyridine, or pyridin-4-amine); and/or
   (6) a potassium channel opener or a selective KCNQ (Kv7) potassium channel opener, or a salt or a salt, hydrate, solvate, tautomer, stereoisomer or deuterated isoform thereof, wherein optionally the selective potassium channel opener comprises flurpirtine (or KATADOLON™), or retigabine (or POTIGA™).

In alternative embodiments of methods and uses as provided herein:
the dosage of the administered drug or drug combination is determined or calculated (either by the individual administering or prescribing the drug or drug combination, or the calculated dosage is provided to the individual administering or prescribing the drug or drug combination) using a dose calculator that calculates an adjusted dose amount equal to the target trough or blood (or plasma) concentration times the moving average (or weighted average) of the current dose/actual trough or Area Under the Curve (AUC) blood (or plasma) drug concentration and prior doses/trough or AUC blood (or plasma) drug concentrations from the previous one to twelve weeks (or months), from two to six weeks (or month) or from three weeks (or months) five weeks (or months), or four weeks (or months);
and optionally the dose calculator further comprises determining the closest available dose amount of the drug or drug combination to the adjusted dose amount, and recommends the available dose amount,
and optionally the dose calculator calculates an adjusted dose amount of the drug or drug combination equal to the target trough or AUC blood (or plasma) concentration times the moving average (or weighted average) of the current dose/actual trough or AUC blood (or plasma) drug concentration and prior doses/trough or AUC blood (or plasma) drug concentrations from the previous one to twelve weeks (or months), from two to six weeks (or months) or from three weeks (or months), and, optionally, excluding the prior trough or AUC drug concentration measurements associated with missed doses and/or transient changes in blood (or plasma) drug concentrations, or before a persistent change in dose amount/trough or AUC blood (or plasma) drug concentration;
the dosage of the administered drug or drug combination is determined or calculated (either by the individual administering or prescribing the drug or drug combination, or the calculated dosage is provided to the individual administering or prescribing the drug or drug combination) using a dose calculator that determines or calculates an adjusted dose amount equal to the target trough or AUCblood (or plasma) concentration times the moving average (or weighted average) of the current dose/actual trough or AUC blood (or plasma) drug concentration, prior doses/trough or AUC blood (or plasma) drug (from zero to twelve previous weeks (or months)), and the starting (or initial) dose/estimated steady state trough blood drug (or plasma) concentration (C_ss, trough) using the following instructions and pharmacokinetic formula: C_ss, trough=(Test Dose/AUC_initial)*τ/(1−e\hat{ }−kτ), where the Test Dose is a no adverse events observed dose amount, a minimal adverse events observed dose amount, a safe dose amount, a safe dose amount based on body surface area, body weight, age, or correlate of a minimal adverse events dose amount, τ=the dosing interval, and k=the elimination rate constant and calculated by (ln(C_last−1)−Ln(C_last))/(T_last−T_last−1) or, optionally, from the slope of the regression line of blood (or plasma) drug concentrations measurements during the terminal elimination phase. Clearance equals the Test Dose/AUC_initial where AUC_initial is calculated as the sum of AUC_0-Last−1, AUC_Last−1-Last and AUC_Last−∞, using the linear trapezoidal rule, log-linear trapezoidal rule and C_n/k, respectively. The Test Dose is administered and 3 to 12 blood samples, 5 to 9 blood samples or 7 blood samples are obtained. The samples are collected at times of about 1 hour, 2 hours, 4 hours, 8 hours, 12 hours, 24 hours, 36 hours, 48 hours, 72 hours, and 96 hours, or about 1 hour, 4 hours, 12 hours, 36 hours and 48 hours, or about 1 hour, 2 hours, 4 hours and 6 hours or about 1 hour, 4 hours and 12 hours or about 1 hour, 2 hours, 4, hours, 12 hours, 24 hours, 48 hours and 72 hours (or at any time therebetween);
the dosage of the administered drug or drug combination is determined or calculated (either by the individual administering or prescribing the drug or drug combination, or the calculated dosage is provided to the individual administering or prescribing the drug or drug combination) using a dose calculator that determines or calculates individualized Starting Dose amount equal to a target trough blood (or plasma) concentration*(times) the Test Dose amount/Test Dose steady-state, and, optionally, a loading dose (or doses) amount equal to a target blood (or plasma) concentration/bioavailability*(times) individual CL/k where bioavailability is determined from the population mean, median, or mode of the percentage bioavailability or, optionally, where the mean can also be the geometric mean. Alternatively, the bioavailability can be described as ranging between about 0.5 to 3.0 standard deviations, or specifically between 1.0 to 2.0 standard deviations, or about 1.5 standard deviations above the average (or geometric mean) bioavailability;
and optionally the dose calculator further comprises determining the closest available dose amount of the drug or drug combination to the adjusted dose amount and recommends the available dose amount,
and optionally the dose calculator further comprises calculating an adjusted dose amount of the drug or drug combination and, optionally, excluding doses and trough or AUC drug concentration measurements associated with missed doses and/or transient change in blood (or plasma) drug concentrations, or before a persistent change in the dose amount/trough or AUC blood (or plasma) drug concentration;
the one drug, or the two or more or all of the drugs in the therapeutic combination, are formulated in a unit dosage amount ranging from between about 0.1 mg, 0.25 mg, 0.5 mg, 1 mg, 2.5 mg, 5 mg, 10 mg, 25 mg, 50 mg, or 100 mg to about 0.5 gram or 1 gram or 2 gram or 4 gram (gm), and optionally the one or two or more or all of the drugs are formulated as an immediate release formulation or a controlled release formulation;
the individual in need thereof is being treated for DMD and the method further comprises administration of a gene therapy comprising use of casimersen (or AMONDYS 45™);
the individual in need thereof is being treated for DMD and the method further comprises administration of a gene therapy comprising use of eteplirsen (or EXONDYS 51™),
the individual in need thereof is being treated for DMD and the method further comprises administration of a gene therapy comprising use of viltolarsen (or VILTEPSO™, or VYONSIA 53™),
the individual in need thereof is being treated for DMD and the method further comprises administration of a gene therapy comprising use of ataluren (or TRANSLARNA™),
the individual in need thereof is being treated for DMD and the method further comprises administration of a gene therapy comprising use of PF-06939926™ (Pfizer),
the individual in need thereof is being treated for DMD and the method further comprises administration of a gene therapy comprising use of SRP-9001™ (Sarepta Therapeutics, Roche),
the individual in need thereof is being treated for DMD and the method further comprises administration of a gene therapy comprising use of SPR-5051™ (Sarepta Therapeutics), or
the individual in need thereof is being treated for DMD and the method further comprises administration of a gene therapy comprising use of DS-5141b™ (Daiichi-Sankyo); and/or
the dosaging of
(a) amifampridine is to attain a predicted blood or plasma Area Under the Curve (AUC) AUC₀₋₄ₕ; target for amifampridine of between about 20 to 300 ng/mL*h, between about 50 to 250 ng/mL*h, between about 100 to 200 ng/mL*h, or 150 ng/mL*h, and, optionally, is to obtain a predicted blood or plasma AUC target precent coefficient of variation between about 5% to 35%, between about 5% to 25, between about 5% to 15%, or between about 5% to 10%;
(b deflazacort (or EMFLAZA™, CALCORT) is to attain a blood or plasma AUC0-4 h target or AUC0-8 h target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or 250 ng/mL*h8; deflazacort is to attain a blood or plasma AUC0-infinity target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or 250 ng/mL*h and, optionally, is to obtain a predicted blood or plasma AUC target precent coefficient of variation between about 5% to 35%, between about 5% to 25, between about 5% to 15% or between about 5% to 10%;
(c) propranolol is to attain a predicted blood or plasma AUC0-9n or AUC0-9h target for propranolol of between about 100 to 1,000 ng/mL*h, between about 200 to 800 ng/mL*h, between about 350 to 600 ng/mL*h or 500 ng/mL*h; or C9h of between about 10 to 100 ng/mL, between about 20 to 80 ng/mL*h, between about 35 to 60 ng/mL*h or 50 ng/mL*h, and, optionally, is to obtain a predicted blood or plasma AUC target precent coefficient of variation between about 5% to 35%, between about 5% to 25, between about 5% to 15%, or between about 5% to 10%;
(d) serabelisib is to attain a predicted blood or plasma AUC0-24 h target for serabelisib of between about 1000 to 25,000 ng/mL*h, between about 3,000 to 20,000 ng/mL*h, between about 5,000 to 15,000 ng/mL*h or 10,000 ng/mL*h, and, optionally, is to obtain a predicted blood or plasma AUC target precent coefficient of variation between about 5% to 35%, between about 5% to 25, between about 5% to 15% or between about 5% to 10%;
(e) sirolimus (also known as rapamycin) is to attain a predicted blood C24h target for sirolimus of between about 1 to 20 ng/mL, between about 5 to 15 ng/ml, between about 10 to 15 ng/mL or 12 ng/mL and, optionally, is to obtain a predicted blood or plasma trough or AUC target precent coefficient of variation between about 5% to 35%, between about 5% to 25, between about 5% to 15%, or between about 5% to 10%;
(f) vamorolone is to attain a predicted blood or plasma AUC0-24 h target for vamorolone of between about 200 to 4000 ng/mL*h, between about 400 to 2,000 ng/mL*h, between about 600 to 1,500 ng/mL*h, between about 800 to 1,200 ng/mL*h or 1,000 ng/mL*h, and, optionally, is to obtain a predicted blood or plasma AUC target precent coefficient of variation between about 5% to 35%, between about 5% to 25, between about 5% to 15%, or between about 5% to 10%;
(g) ataluren is to attain a predicted blood or plasma C6 h target or a trough blood or plasma concentration target prior to the evening dose for ataluren of between about 1 to 20 μg/mL, between about 2 to 15 μg/mL, between about 5 to 10 μg/mL or 7 μg/mL, or to attain a predicted blood AUC₀₋₆ target for ataluren of between about 10 to 1,000 μg/mL*h, between about 20 to 500 μg/mL*h, between about 300 to 300 μg/mL*h or between about 40 to 200 μg/mL*h or 100 μg/mL*h, and, optionally, is to obtain a predicted blood or plasma trough or AUC target precent coefficient of variation between about 5% to 35%, between about 5% to 25, between about 5% to 15%, or between about 5% to 10%; or,
(h) everolimus is to attain a predicted blood C₁₂ₕ or C₂₄ₕ target for everolimus of between about 0.5 to 40 ng/mL, between about 1 to 20 ng/mL, between about 5 to 15 ng/ml, between about 10 to 15 ng/mL or 12 ng/mL, or to attain a predicted blood AUC₀₋₁₂ or AUC₀₋₂₄ target for everolimus of between about 50 to 3,000 ng*/mL*h, between about 100 to 1,500 ng/mL*h, between about 200 to 1,200 ng/mL*h or between about 300 to 1000 ng/mL*h or 500 ng/mL*h, and, optionally, is to obtain a predicted blood or plasma trough or AUC target precent coefficient of variation between about 5% to 50%, between about 5% to 35%, between about 5% to 25, between about 5% to 15% or between about 5% to 10%,
wherein optionally a dosaging calculation for any of (a) to (f) is provided to an individual who is administering the one drug, or the two or more or all of the drugs in the therapeutic combination.

In alternative embodiments, provide are uses of:
(a) a drug or a therapeutic combination of drugs and a companion diagnostic as provided herein; or
(b) at least one drug comprising:
(1) a beta adrenergic blocker (or beta blocker), or a salt, hydrate, solvate, tautomer, stereoisomer or deuterated isoform thereof, wherein optionally the beta adrenergic blocker comprises propranolol (or INDERAL™) atenolol (or TENORMIN™), metoprolol (or LOPRESOR™), a carvedilol (or COREG™), a Bisoprolol (or ZEBETA™), nadolol (or CORGARD™), a nebivolol (or BISTOLIC™), timolol (or TIMOL™) or a combination thereof;
(2) a glucocorticoid, or a salt, hydrate, solvate, tautomer, stereoisomer or deuterated isoform thereof, wherein optionally the glucocorticoid comprises deflazacort (or EMFLAZA™, CALCORT™), vamorolone (or 17α,21-Dihydroxy-16α-methylpregna-1,4,9(11)-triene-3,20-dione), prednisone (or DELTASONE™, LIQUID PRED™, ORASONE™) prednisolone (or ORAPRED™, PEDIAPRED™, MILLIPRED™) or a combination thereof.
(3) an mTOR (mammalian target of rapamycin) inhibitor, or a salt, hydrate, solvate, tautomer, stereoisomer or deuterated isoform thereof, wherein optionally the mTOR inhibitor comprises everolimus (or AFINITOR™ ZORTRESS™), sirolimus (also known as rapamycin) (or RAPAMUNE™ FYARRO™) or a combination thereof,
(4) a phosphatidylinositol-4,5-bisphosphate 3-kinase, catalytic subunit alpha inhibitor (or PIK3Ca inhibitor, or p110α protein), or a salt, hydrate, solvate, tautomer, stereoisomer or deuterated isoform thereof, wherein optionally the PIK3Ca inhibitor comprise alpelisib (or PIQRAY™ VIJOICE™), inavolisib (or GDC-0077 (Genentech)), serabelisib (or [6-(2-amino-1,3-benzoxazol-5-yl)imidazo[1,2-a]pyridin-3-yl]-morpholin-4-ylmethanone), CYH-33, MN1611 or a combination thereof, and
(5) a voltage-gated potassium channel blocker, or a salt, hydrate, solvate, tautomer, stereoisomer or deuterated isoform thereof, wherein optionally the voltage-gated potassium channel blocker comprises amifampridine (or for example FIRDAPSE™, RUZURGI™), dalfampridine (or 4-aminopyridine, or pyridin-4-amine);
(6) a mineralocorticoid receptor antagonist, or a salt, hydrate, solvate, tautomer, stereoisomer or deuterated isoform thereof, wherein optionally the mineralocorticoid receptor antagonist comprises spironolactone (for example, ALDACTONE™, SPIRACTIN™, VEROSPIRON™) or eplerenone (for example, INSPRA™, EPNONE™, DOSTEREP™);
(7) a 4-phenylbutyric acid, or a salt, hydroate, solvate, tutomer, steroisomer or deuterated isoform thereof, wherein optional the 4-phenylbutyric acid is a glycerol phenylbutyrate (for example, RAVICTI™), sodium phenylbutyrate (for example, BUPHENYL™);
(8) a protein kinase inhibitor, or a salt, hydrate, solvate, tautomer, stereoisomer or deuterated isoform thereof, wherein optionally the protein kinase inhibitor is an abemaciclib (or VERZENIO™, VERZENIOS™), an acalabrutinib (or CALQUENCE™), an afatinib, an alectinib, a avapritinib, an axitinib, a binimetinib, a baricitinib, a binimetinib, a bosutinib, a brigatinib, a cabozantinib, a ceritinib, a capmatinib, a cobimetinib, a copanlisib, a crizotinib, a dabrafenib, a dacomitinib, a dasatinib, a duvelisib, an erdafinib, an encorafenib, an erlotinib, a fasudil, fedratinib, a filgotinib, a fostamatinib, a gefitinib, a gilteritinib, an ibrutinib, am idelalisib, an imatinib, an infigratinib, a lapatinib, a larotrectinib, a lenvatinib, a lestaurtinib, a lorlatinib, a masitinib, a midostaurin, a momelotinib, a neratinib, a netarsudil, a nilotinib, a nintedanib, an oclacitinib, an osimertinib, a pacritinib, a palbociclib, a pazopanib, a peficitinib, a pexidartinb, a ponatinib, a regorafenib, a ribociclib, a ruxolitinib, a selumetinib, a sorafenib, a sunitinib, a tepotinib, a tivozanib, a tofacitinib, a trametinib, a tucatinib, a upadacitinib, a vandetanib, a vemurafenib, a zanubrutinib, or any combination thereof;
(9) an immunomodulatory drugs (iMiDs) or a salt, hydrate, solvate, tautomer, stereoisomer or deuterated isoform thereof, wherein optionally the immune-modulatory drug comprises: a lenalidomide, a pomalidomide, a thalidomide, or any combination thereof;
(10) a sphingosine-1-phosphate receptor modulator or a salt, hydrate, solvate, tautomer, stereoisomer or deuterated isoform thereof, wherein optionally the sphingosine-1-phosphate receptor modulators is a fingolimod, an ozanimod, a ponesimod, or any combination thereof;
(11) a siponimod, a calcineurin inhibitor, or a salt, hydrate, solvate, tautomer, stereoisomer or deuterated isoform thereof, wherein optionally the calcineurin inhibitor comprises tacrolimus, cyclosporin, or any combination thereof;
(12) an inosine-5′-monophosphate dehydrogenase inhibitor, or a salt, hydrate, solvate, tautomer, stereoisomer or deuterated isoform thereof, wherein optionally the inosine-5′-monophosphate dehydrogenase inhibitor comprises a mycophenolate mofetile;
(13) cyclooxygenase-2 selective non-steroidal anti-inflammatory drugs or a salt, hydrate, solvate, tautomer, stereoisomer or deuterated isoform thereof, wherein optionally the cyclooxygenase-2 selective non-steroidal anti-inflammatory drugs is a celecoxib, an etodolac, an etoricoxib, a meloxicam, a rofecoxib, a valdecoxib, or any combination thereof,
(14) lithium drug or a salt, hydrate, solvate, tautomer, stereoisomer or a deuterated isoform thereof, wherein optionally the lithium salt comprises a lithium carbonate, lithium acetate, lithium sulfate, lithium citrate, lithium orotate, lithium gluconate or any combination thereof,
(15) an angiotensin converting enzyme inhibitor, an enalapril (or VASCOTECH™), a lisinopril (or PRINIVIL™ or ZESTRIL™), a perindopril (or ACEON™),
(16) an angiotensin receptor blocker, a losartan (or COZAAR™)
(17) an antiepileptic drug, alprazolam (or XANAX™), cannabidiol (or EPIDIOLEX™), carbamazepine (or TEGRETOL™, CARBATROL™) clobazam (or FRISIUM™, ONFI™), Clonazepam (or KLONOPIN™ RIVOTRIL™), diazepam (or VALIUM™), ethosuximide (or ZARONTIN™), fenfluramine (or FINTEPLA™), Flurpirtine (or KATADOLON™), gabapentin (or NEURONTIN™, GRALISE™) Ganaxolone (or GANAXONE™, ZTALMY™), Lacosamide (or VIMIPAT™), Lorazepam (or ATIVAN™), Levetiracetam (or KEPPRA™ ROWEEPRA™), Lamotrigine (or LAMICTAL™), Memantine (or NAMENDA™), Methsuximide (or CELONTIN™), Oxcarbazepine (or TRILEPTAL™, OXTILAR XR™), Perampanel (or FYCOMPA™), Pyridoxine (or vitamin B6), Phenobarbital (or LUMINAL™), Phenytoin (or DILANTIN™, EPTOIN™), Retigabine (or POTIGA™ TROBALT™), Rufinamide (or BANZEL™, INOVELON™), Stiripentol (or DIACOMIT™), Topiramate (or TOPAMAX™, TROKENDI XR™) Valproic Acid (or DEPAKENE™, DEPAKOTE™), Vigabatrin (or SABRIL™), Vinpocetine (or CAVINTON™, INTELECTOL™) Zonisamide (or ZONEGRAN™) or any combination thereof,
(18) an oxadiazole capable of making a ribosome less sensitive to premature stop codons (an effect referred to as “read-through”) by promoting insertion of near-cognate tRNA at nonsense codons, or ataluren or 3-[5-(2-Fluorophenyl)-1,2,4-oxadiazol-3-yl]benzoic acid, or TRANSLARNA™ and/or
(19) a colchicine (or COLCRYS™, GLOPERBA™, MITIGARE™),
to manufacture a medicament, optionally to manufacture a medicament for treating, ameliorating, preventing, slowing the advance of, or lessoning the symptoms of:
a vascular anomaly,
   and optionally the vascular anomaly comprises infantile hemangioma, Arteriovenous Malformation (AVM), Cerebral Cavernous Malformation (CCM), Hereditary Hemorrhagic Telangiectasia (HHT), Von Hippel-Lindau Disease (VHL), angiosarcoma, Kaposiform Hemangioendothelioma (KHE), Kaposiform Lymphangioendothelioma (KLA), Microcystic Lymphatic Malformations or a PIKCA-Related Overgrowth Spectrum (PROS),
   and optionally the PROS or vascular anomaly is: Fibro-adipose vascular anomaly Hemihyperplasia—multiple lipomatosis syndrome, CLOVES (congenital lipomatous (fatty) overgrowth, vascular malformations, epidermal nevi and scoliosis/skeletal/spinal anomalies) syndrome Macrodactyly, Facial infiltrating lipomatosis, Macrocephaly-capillary malformation, Dysplastic megalencephaly or Klippel-Trénaunay syndrome;
a Muscular Dystrophy (MD),
   and optionally the MD is Duchenne Muscular Dystrophy (DMD), Beckers Muscular Dystrophy, Congenital Muscular Dystrophy, Facioscapulohumeral Dystrophy, Limb-Girdle Muscular Dystrophy (LGMD), Myotonic Dystrophy Type 1, Myotonic Dystrophy Type 2, or Lambert Eaton Myasthenic Syndrome (LEMS);
a nervous system disorder,
   and optionally the nervous system disorder is Huntington's Disease, Tuberous Sclerosis Complex (TSC), or subependymal giant cell astrocytoma (SEGA) associated with TSC, or a developmental Epileptic Encephalopathy, a Dravet Syndrome, an Epilepsy in Infancy with Migrating Focal Seizures, a Landau Kleffner Syndrome, a Lennox-Gastaut Syndrome (LGS), a Myoclonic Atonic Epilepsy (Doose Syndrome), a Rett Syndrome, a West Syndrome, or, Epilepsies Associated with Specific Genetic or Metabolic Disorders: a GLUT1 Deficiency Syndrome, a Neuronal Ceroid Lipofuscinosis (NCL), a Protocadherin 19 Female-Limited Epilepsy (PCDH19 Epilepsy), Pyridoxamine 5′-Phosphate Oxidase Deficiency, Trisomy 21 and Epileptic Spasms, TSC associated seizures or other rare or refractory epilepsies optionally comprising Epilepsy with Continuous Spike-Wave during Sleep (CSWS): Febrile Infection-Related Epilepsy Syndrome (FIRES) and medically intractable epilepsies, a complex partial seizures, infantile spasms, transitional focal epilepsy in infancy, a status epilepticus, partial seizures, catamenial epilepsies, early myoclonic encephalopathy, persistent spike epilepsy in slow wave sleep (except Landau-Kleffner syndrome), hypothalamic epilepsy, dose syndrome (myoclonic-standing disability epilepsy), myoclonic states of non-progressive encephalopathy, tawnian syndrome or early epileptic encephalopathy in infancy, glycine encephalopathy, 15q repeat syndrome (Dup 15 q), epilepsy associated with CHD2, cyclin-dependent kinase-like 5 (CDKL 5), SCN1A, SCN2A, SCN8A, ARX, KCNA1, KCNA2, KCNT1, KCNQ2, HCN1, PCDH19, GRIN1, GRIN2A and GRIN2B mutations, a Smith-Magenis Syndrome, 1p36 deletion syndrome, 16p11.2 deletion syndrome, fragile X syndrome, trisomy 21, Angelman's Syndrome, Schaff-Yang Syndrome, Albright Hereditary Osteodystroph, Silver-Russell Syndrome, Alstrom Syndrome, Mental Retardation or WAGR or WAGRO Syndrome; Gillespie syndrome or cerebellar ataxia, Coffin-Lowry Syndrome, hypersomnia, excessive daytime sleepiness (EDS), excessive daytime sleepiness associated with central or obstructive sleep apnea, idiopathic hypersomnia, sleep related breathing disorders, obstructive sleep apnea, central sleep apnea, Sleep-Related Hypoventilation Disorders, Sleep-Related Hypoxemia disorder, Rapid eye movement (REM) sleep behavior disorder, restless leg syndrome, nightmare disorder, dream anxiety disorder, Alpers Disease, Alpers Syndrome, Kearns-Sayre syndrome (KSS). Leber hereditary optic neuropathy (LHON), Leigh disease, Leigh syndrome, Mitochondrial encephalopathy lactic acidosis and stroke-like episodes (MELAS), Myoclonic epilepsy and ragged-red fiber disease syndrome (MERRF), Pontocerebellar Hypoplasia Type 6 (PCH6), Autism or Autism Spectrum Disorder (ASD), Asperger spectrum, or a Pervasive Developmental Disorder (PDD), hypersomnia, excessive daytime sleepiness (EDS), excessive daytime sleepiness associated with central or obstructive sleep apnea, idiopathic hypersomnia, narcolepsy, narcolepsy type 1, sleep related breathing disorders, obstructive sleep apnea, central sleep apnea, Sleep-Related Hypoventilation Disorders, Sleep-Related Hypoxemia Disorder, Rapid Eye Movement (REM) sleep behavior disorder, restless leg syndrome (RLS), nightmare disorder, dream anxiety disorder or a chronic fatigue syndrome, Parkinson's Disease and Alzheimer's Disease;
A neuropsychiatric disorder,
   and optionally the neuropsychiatric disorder is TSC-Associated Neuropsychiatric Disorder (TAND), General Anxiety, Social Anxiety Disorder (Social Phobia), attention deficit hyperactivity disorder (ADHD) disruptive mood dysregulation disorder (DMDD), intermittent explosive disorder (IED), major depressive disorder (MDD), treatment resistant depression (TRD), obsessive-compulsive disorder (OCD), negative symptoms of schizophrenia, Parkinson's Disease, MDD Associated with Parkinson's Disease, general anxiety, Social Anxiety Disorder (Social Phobia), oppositional defiant disorder (ODD);
a retinopathy,
   and optionally the retinopathy is a retinopathy of prematurity (ROP), Norrie disease retinopathy, Familial Exudative Vitreoretinopathy (FEVR), Coats' disease retinopathy or diabetic retinopathy;
a cardiomyopathy,
   and optionally the cardiomyopathy is infantile hypertrophic cardiomyopathy, infantile dilated cardiomyopathy, infantile restricted cardiomyopathy, Barth's syndrome, mitochondrial cardiomyopathy;
a renal disease, and optionally, the renal disease is nephrotic syndrome;
a peripheral nervous system disorder,
   and optionally the peripheral nervous system disorder is Hereditary Neuropathy with liability to Pressure Palsies (HNPP), Neurofibromatosis Type 1 (NF1), Neurofibromatosis Type 2 (NF2), Schwannoma;
a neuroendocrine system disorder,
   and optionally the neuroendocrine system disorder is Pheochromocytomas (PHEO), paragangliomas (PGL), pheochromocytomas and paragangliomas (PPGL), Hereditary paraganglioma-pheochromocytoma (PGL/PCC).
an allograph organ rejection prophylaxis, and optionally receiving allograft renal transplants, allograft liver transplants, allograft heart transplant;
a benign tumor, and wherein optionally the benign tumor is tuberous sclerosis complex (TSC)-associated subependymal giant cell astrocytoma (SEGA), cardiac rhabdomyoma, renal angiomyolipoma, PTEN hamartoma tumor syndrome (PHTS), Bannayan-Riley Ruvalcaba Syndrome (BRRS), Birt-Hogg-Dube Syndrome, Cowden's Syndrome, Proteus Syndrome or PTEN-related Proteus Syndrome;
a malignant neoplasm or cancer, wherein optionally the malignant neoplasm is a breast cancer, a hormone receptor positive breast tumor, a pancreatic neuroendocrine (pNET) or a renal cell carcinoma (RCC);
an autoimmune disorder, wherein optionally the autoimmune disorder is Crohn's Disease, Diabetes Type 1, Multiple Sclerosis, Myasthenia Gravis, Rheumatoid Arthritis, Lupus, Scleroderma and/or Psoriasis,
a dermatological disorder, for example, psoriasis, urticaria or angioedema;
a pulmonary disease, wherein optionally the pulmonary disease is a lymphangioleiomyomatosis (LAM); and/or is a pulmonary arterial hypertension (PAH); and/or
a polyposis, wherein optionally the polyposis is a familial adenomatous polyposis (FAP) or hereditary flat adenoma syndrome or a Juvenile polyposis syndrome (JPS).

In alternative embodiments, provided are drugs or therapeutic combinations of drugs and a companion diagnostic as provided herein; or, at least one drug comprising:
(1) a beta adrenergic blocker (or beta blocker), or a salt, hydrate, solvate, tautomer, stereoisomer or deuterated isoform thereof, wherein optionally the beta adrenergic blocker comprises propranolol (or INDERAL™) atenolol (or TENORMIN™), metoprolol (or LOPRESOR™), a carvedilol (or COREG™), a Bisoprolol (or ZEBETA™), nadolol (or CORGARD™), a nebivolol (or BISTOLIC™), timolol (or TIMOL™) or a combination thereof;
(2) a glucocorticoid, or a salt, hydrate, solvate, tautomer, stereoisomer or deuterated isoform thereof, wherein optionally the glucocorticoid comprises deflazacort (or EMFLAZA™, CALCORT™), vamorolone (or 17a,21-Dihydroxy-16α-methylpregna-1,4,9(11)-triene-3,20-dione), prednisone (or DELTASONE™, LIQUID PRED™, ORASONE™) prednisolone (or ORAPRED™, PEDIAPRED™, MILLIPRED™) or a combination thereof.
(3) an mTOR (mammalian target of rapamycin) inhibitor, or a salt, hydrate, solvate, tautomer, stereoisomer or deuterated isoform thereof, wherein optionally the mTOR inhibitor comprises everolimus (or AFINITOR™ ZORTRESS™), sirolimus (also known as rapamycin) (or RAPAMUNE™ FYARRO™) or a combination thereof,
(4) a phosphatidylinositol-4,5-bisphosphate 3-kinase, catalytic subunit alpha inhibitor (or PIK3Ca inhibitor, or p110a protein), or a salt, hydrate, solvate, tautomer, stereoisomer or deuterated isoform thereof, wherein optionally the PIK3Ca inhibitor comprise alpelisib (or PIQRAY™ VIJOICE™), inavolisib (or GDC-0077 (Genentech)), serabelisib (or [6-(2-amino-1,3-benzoxazol-5-yl)imidazo[1,2-a]pyridin-3-yl]-morpholin-4-ylmethanone), CYH-33, MN1611 or a combination thereof; and
(5) a voltage-gated potassium channel blocker, or a salt, hydrate, solvate, tautomer, stereoisomer or deuterated isoform thereof, wherein optionally the voltage-gated potassium channel blocker comprises amifampridine (or for example FIRDAPSE™, RUZURGI™), dalfampridine (or 4-aminopyridine, or pyridin-4-amine);
(6) a mineralocorticoid receptor antagonist, or a salt, hydrate, solvate, tautomer, stereoisomer or deuterated isoform thereof, wherein optionally the mineralocorticoid receptor antagonist comprises spironolactone (for example, ALDACTONE™, SPIRACTIN™, VEROSPIRON™) or eplerenone (for example, INSPRA™, EPNONE™, DOSTEREP™);
(7) a 4-phenylbutyric acid, or a salt, hydroate, solvate, tutomer, steroisomer or deuterated isoform thereof, wherein optional the 4-phenylbutyric acid is a glycerol phenylbutyrate (for example, RAVICTI™), sodium phenylbutyrate (for example, BUPHENYL™);
(8) a protein kinase inhibitor, or a salt, hydrate, solvate, tautomer, stereoisomer or deuterated isoform thereof, wherein optionally the protein kinase inhibitor is an abemaciclib (or VERZENIO™, VERZENIOS™), an acalabrutinib (or CALQUENCE™), an afatinib, an alectinib, a avapritinib, an axitinib, a binimetinib, a baricitinib, a binimetinib, a bosutinib, a brigatinib, a cabozantinib, a ceritinib, a capmatinib, a cobimetinib, a copanlisib, a crizotinib, a dabrafenib, a dacomitinib, a dasatinib, a duvelisib, an erdafinib, an encorafenib, an erlotinib, a fasudil, fedratinib, a filgotinib, a fostamatinib, a gefitinib, a gilteritinib, an ibrutinib, am idelalisib, an imatinib, an infigratinib, a lapatinib, a larotrectinib, a lenvatinib, a lestaurtinib, a lorlatinib, a masitinib, a midostaurin, a momelotinib, a neratinib, a netarsudil, a nilotinib, a nintedanib, an oclacitinib, an osimertinib, a pacritinib, a palbociclib, a pazopanib, a peficitinib, a pexidartinb, a ponatinib, a regorafenib, a ribociclib, a ruxolitinib, a selumetinib, a sorafenib, a sunitinib, a tepotinib, a tivozanib, a tofacitinib, a trametinib, a tucatinib, a upadacitinib, a vandetanib, a vemurafenib, a zanubrutinib, or any combination thereof;
(9) an immunomodulatory drugs (iMiDs) or a salt, hydrate, solvate, tautomer, stereoisomer or deuterated isoform thereof, wherein optionally the immune-modulatory drug comprises: a lenalidomide, a pomalidomide, a thalidomide, or any combination thereof;
(10) a sphingosine-1-phosphate receptor modulator or a salt, hydrate, solvate, tautomer, stereoisomer or deuterated isoform thereof, wherein optionally the sphingosine-1-phosphate receptor modulators is a fingolimod, an ozanimod, a ponesimod, or any combination thereof;
(11) a siponimod, a calcineurin inhibitor, or a salt, hydrate, solvate, tautomer, stereoisomer or deuterated isoform thereof, wherein optionally the calcineurin inhibitor comprises tacrolimus, cyclosporin, or any combination thereof;
(12) an inosine-5′-monophosphate dehydrogenase inhibitor, or a salt, hydrate, solvate, tautomer, stereoisomer or deuterated isoform thereof, wherein optionally the inosine-5′-monophosphate dehydrogenase inhibitor comprises a mycophenolate mofetile;
(13) cyclooxygenase-2 selective non-steroidal anti-inflammatory drugs or a salt, hydrate, solvate, tautomer, stereoisomer or deuterated isoform thereof, wherein optionally the cyclooxygenase-2 selective non-steroidal anti-inflammatory drugs is a celecoxib, an etodolac, an etoricoxib, a meloxicam, a rofecoxib, a valdecoxib, or any combination thereof,
(14) lithium drug or a salt, hydrate, solvate, tautomer, stereoisomer or a deuterated isoform thereof, wherein optionally the lithium salt comprises a lithium carbonate, lithium acetate, lithium sulfate, lithium citrate, lithium orotate, lithium gluconate or any combination thereof,
(15) an angiotensin converting enzyme inhibitor, an enalapril (or VASCOTECH™), a lisinopril (or PRINIVIL™ or ZESTRIL™), a perindopril (or ACEON™)
(16) an angiotensin receptor blocker, a losartan (or COZAAR™)
(17) an antiepileptic drug, alprazolam (or XANAX™), cannabidiol (or EPIDIOLEX™), carbamazepine (or TEGRETOL™, CARBATROL™) clobazam (or FRISIUM™, ONFI™), Clonazepam (or KLONOPIN™ RIVOTRIL™), diazepam (or VALIUM™), ethosuximide (or ZARONTIN™), fenfluramine (or FINTEPLA™), Flurpirtine (or KATADOLON™), gabapentin (or NEURONTIN™, GRALISE™) Ganaxolone (or GANAXONE™, ZTALMY™), Lacosamide (or VIMIPAT™), Lorazepam (or ATIVAN™), Levetiracetam (or KEPPRA™ ROWEEPRA™), Lamotrigine (or LAMICTAL™), Memantine (or NAMENDA™), Methsuximide (or CELONTIN™), Oxcarbazepine (or TRILEPTAL™, OXTILAR XR™), Perampanel (or FYCOMPA™), Pyridoxine (or vitamin B6), Phenobarbital (or LUMINAL™), Phenytoin (or DILANTIN™, EPTOIN™), Retigabine (or POTIGA™ TROBALT™), Rufinamide (or BANZEL™, INOVELON™), Stiripentol (or DIACOMIT™), Topiramate (or TOPAMAX™, TROKENDI XR™) Valproic Acid (or DEPAKENE™, DEPAKOTE™), Vigabatrin (or SABRIL™), Vinpocetine (or CAVINTON™, INTELECTOL™) Zonisamide (or ZONEGRAN™) or any combination thereof,
(18) an oxadiazole capable of making a ribosome less sensitive to premature stop codons (an effect referred to as “read-through”) by promoting insertion of near-cognate tRNA at nonsense codons, or ataluren or 3-[5-(2-Fluorophenyl)-1,2,4-oxadiazol-3-yl]benzoic acid, or TRANSLARNA™ and/or
(19) a colchicine (or COLCRYS™, (GLOPERBA™, MITIGARE™),
for use in treating, ameliorating, preventing, slowing the advance of, or lessoning the symptoms of:
a vascular anomaly,
   and optionally the vascular anomaly comprises infantile hemangioma, Arteriovenous Malformation (AVM), Cerebral Cavernous Malformation (CCM), Hereditary Hemorrhagic Telangiectasia (HHT), Von Hippel-Lindau Disease (VHL), angiosarcoma, Kaposiform Hemangioendothelioma (KHE), Kaposiform Lymphangioendothelioma (KLA). Microcystic Lymphatic Malformations or a PIKCA-Related Overgrowth Spectrum (PROS),
   and optionally the PROS is: Fibro-adipose vascular anomaly Hemihyperplasia—multiple lipomatosis syndrome, CLOVES (congenital lipomatous (fatty) overgrowth, vascular malformations, epidermal nevi and scoliosis/skeletal/spinal anomalies) syndrome Macrodactyly, Facial infiltrating lipomatosis, Macrocephaly-capillary malformation, Dysplastic megalencephaly or Klippel-Trenaunay syndrome;
a Muscular Dystrophy (MD),
   and optionally the MD is Duchenne Muscular Dystrophy (DMD), Beckers Muscular Dystrophy, Congenital Muscular Dystrophy, Facioscapulohumeral Dystrophy, Limb-Girdle Muscular Dystrophy (LGMD), Myotonic Dystrophy Type 1, Myotonic Dystrophy Type 2, or Lambert Eaton Myasthenic Syndrome (LEMS);
a nervous system disorder,
   and optionally the nervous system disorder is Huntington's Disease, Tuberous Sclerosis Complex (TSC), or subependymal giant cell astrocytoma (SEGA) associated with TSC, or a developmental and Epileptic Encephalopathies, a Dravet Syndrome, a Epilepsy in Infancy with Migrating Focal Seizures. a Landau Kleffner Syndrome, a Lennox-Gastaut Syndrome (LGS), a Myoclonic Atonic Epilepsy (Doose Syndrome), a Rett Syndrome, a West Syndrome, or, Epilepsies Associated with Specific Genetic or Metabolic Disorders: a GLUT1 Deficiency Syndrome, a Neuronal Ceroid Lipofuscinosis (NCL), a Protocadherin 19 Female-Limited Epilepsy (PCDH19 Epilepsy), Pyridoxamine 5′-Phosphate Oxidase Deficiency, Trisomy 21 and Epileptic Spasms, TSC associated seizures or other rare or refractory epilepsies including Epilepsy with Continuous Spike-Wave during Sleep (CSWS): Febrile Infection-Related Epilepsy Syndrome (FIRES) and medically intractable epilepsies, a complex partial seizures, infantile spasms, transitional focal epilepsy in infancy, a status epilepticus, partial seizures, catamenial epilepsies, early myoclonic encephalopathy, persistent spike epilepsy in slow wave sleep (except Landau-Kleffner syndrome), hypothalamic epilepsy, dose syndrome (myoclonic-standing disability epilepsy), myoclonic states of non-progressive encephalopathy, tawnian syndrome or early epileptic encephalopathy in infancy, glycine encephalopathy, 15q repeat syndrome (Dup 15 q), epilepsy associated with CHD2, cyclin-dependent kinase-like 5 (CDKL 5), SCN1A, SCN2A, SCN8A, ARX, KCNA1, KCNA2, KCNT1, KCNQ2, HCN1, PCDH19, GRIN1, GRIN2A and GRIN2B mutations, a Smith-Magenis Syndrome, 1p36 deletion syndrome, 16p11.2 deletion syndrome, fragile X syndrome, trisomy 21, Angelman's Syndrome, Schaff-Yang Syndrome, Albright Hereditary Osteodystroph, Silver-Russell Syndrome, Alstrom Syndrome, Mental Retardation or WAGR or WAGRO Syndrome; Gillespie syndrome or cerebellar ataxia, Coffin-Lowry Syndrome, hypersomnia, excessive daytime sleepiness (EDS), excessive daytime sleepiness associated with central or obstructive sleep apnea, idiopathic hypersomnia, sleep related breathing disorders, obstructive sleep apnea, central sleep apnea, Sleep-Related Hypoventilation Disorders, Sleep-Related Hypoxemia disorder, Rapid eye movement (REM) sleep behavior disorder, restless leg syndrome, nightmare disorder, dream anxiety disorder, Alpers Disease, Alpers Syndrome, Kearns-Sayre syndrome (KSS). Leber hereditary optic neuropathy (LHON), Leigh disease, Leigh syndrome, Mitochondrial encephalopathy lactic acidosis and stroke-like episodes (MELAS), Myoclonic epilepsy and ragged-red fiber disease syndrome (MERRF), Pontocerebellar Hypoplasia Type 6 (PCH6), Autism or Autism Spectrum Disorder (ASD), Asperger spectrum, or a Pervasive Developmental Disorder (PDD), hypersomnia, excessive daytime sleepiness (EDS), excessive daytime sleepiness associated with central or obstructive sleep apnea, idiopathic hypersomnia, narcolepsy, narcolepsy type 1, sleep related breathing disorders, obstructive sleep apnea, central sleep apnea, Sleep-Related Hypoventilation Disorders, Sleep-Related Hypoxemia Disorder, Rapid Eye Movement (REM) sleep behavior disorder, restless leg syndrome (RLS), nightmare disorder, dream anxiety disorder or a chronic fatigue syndrome, Parkinson's Disease and Alzheimer's Disease;
A neuropsychiatric disorder,
   and optionally the neuropsychiatric disorder is TSC-Associated Neuropsychiatric Disorder (TAND), General Anxiety, Social Anxiety Disorder (Social Phobia), attention deficit hyperactivity disorder (ADHD) disruptive mood dysregulation disorder (DMDD), intermittent explosive disorder (IED), major depressive disorder (MDD), treatment resistant depression (TRD), obsessive-compulsive disorder (OCD), negative symptoms of schizophrenia, Parkinson's Disease, MDD Associated with Parkinson's Disease, oppositional defiant disorder (ODD);
a retinopathy,
   and optionally the retinopathy is a retinopathy of prematurity (ROP), Norrie disease retinopathy, Familial Exudative Vitreoretinopathy (FEVR), Coats' disease retinopathy or diabetic retinopathy;
a cardiomyopathy,
   and optionally the cardiomyopathy is infantile hypertrophic cardiomyopathy, infantile dilated cardiomyopathy, infantile restricted cardiomyopathy, Barth's syndrome, mitochondrial cardiomyopathy;
a renal disease, and optionally, the renal disease is nephrotic syndrome;
a peripheral nervous system disorder,
   and optionally the peripheral nervous system disorder is Hereditary Neuropathy with liability to Pressure Palsies (HNPP), Neurofibromatosis Type 1 (NF1), Neurofibromatosis Type 2 (NF2), Schwannoma;
a neuroendocrine system disorder,
   and optionally the neuroendocrine system disorder is Pheochromocytomas (PHEO), paragangliomas (PGL), pheochromocytomas and paragangliomas (PPGL), Hereditary paraganglioma-pheochromocytoma (PGL/PCC);
an allograph organ rejection prophylaxis, and optionally receiving allograft renal transplants, allograft liver transplants, allograft heart transplant;
a benign tumor, and wherein optionally the benign tumor is tuberous sclerosis complex (TSC)-associated subependymal giant cell astrocytoma (SEGA), cardiac rhabdomyoma, renal angiomyolipoma, PTEN hamartoma tumor syndrome (PHTS), Bannayan-Riley Ruvalcaba Syndrome (BRRS), Birt-Hogg-Dube Syndrome, Cowden's Syndrome, Proteus Syndrome or PTEN-related Proteus Syndrome;
a malignant neoplasm or cancer, wherein optionally the malignant neoplasm is a breast cancer, a hormone receptor positive breast tumor, a pancreatic neuroendocrine (pNET) or a renal cell carcinoma (RCC);
an autoimmune disorder, wherein optionally the autoimmune disorder is Crohn's Disease, Diabetes Type 1, Multiple Sclerosis, Myasthenia Gravis, Rheumatoid Arthritis, Lupus, Scleroderma and/or Psoriasis;
a dermatological disorder, for example, psoriasis, urticaria or angioedema;
a pulmonary disease, wherein optionally the pulmonary disease is a lymphangioleiomyomatosis (LAM); and/or is pulmonary arterial hypertension (PAH); and/or
a polyposis, wherein optionally the polyposis is a familial adenomatous polyposis (FAP) or hereditary flat adenoma syndrome or a Juvenile polyposis syndrome (JPS).

In alternative embodiments, provided are products of manufacture comprising or manufactured or fabricated as: a kit, a plurality of packages or packettes, a sachet, a blister packet, lidded blister or blister card or packets, or a shrink wrap, comprising or having contained or packaged therein a drug or a therapeutic combination of drugs, and companion diagnostic as provided herein, wherein optionally the kit, plurality of packages or packettes, blister packet, lidded blister or blister card or packets, or shrink wrap comprises a separate container for each drug contained in the kit, plurality of packages or packettes, blister packet, lidded blister or blister card or packet, or shrink wrap, and each separate container contains or comprise a unique QR code the identifies the package and the individual drug contained in the separate container, and optionally each separate container is associated with a time stamp that record when each of the separate containers is opened (and dispensed or given to the individual in need thereof, or patient).

In alternative embodiments, the products of manufacture, or kits, sachets, a plurality of packages or packettes, blister packets, lidded blisters, blister cards, packets or a shrink wraps, as provided herein further comprises a time stamp device for each drug container or receptacle, or a time stamp device operably connected to or associated with each drug container or receptacle,
and optionally the time stamp device records, transmits to a remote device, or displays to a user as a readable output data comprising when (the time) each of the separate containers is opened, and optionally data comprising how often each of the separate containers is opened, and optionally the time stamp device data is transmitted to the user, or a patient, a caregiver, a technician, a nurse and/or a physician, or optionally to a central database or to a remote computer or device,
and optionally each (separate) drug container or receptable comprises a unique Quick Response (QR) code, or unique two-dimensional matrix barcode, to identify each drug container or receptable and each drug or drugs contained in each drug container or receptable.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The drawings set forth herein are illustrative of exemplary embodiments provided herein and are not meant to limit the scope of the invention as encompassed by the claims.

FIG. 1 is a flow diagram of an exemplary method for generating a moving average of administered drug blood concentrations or a PK profile of a particular patient in accordance with an example embodiment of the present disclosure, as discussed in further detail, below.

FIG. 2 illustrates an architecture of an exemplary MIPD system; the MIPD business logic is contained in a PKPD-engine, which is implemented as a service that can be called by the main application, as discussed in further detail, below.

FIG. 3 is a flow diagram of an exemplary method for generating a drug dose recommendation of a particular patient in accordance with an example embodiment of the present disclosure, including use of a companion diagnostic such as a dried blood spot, a volumetric dried blood spot, a volumetric absorptive microsampling (VAMS), or an at-home high-volume liquid blood collection device, and shipping the collected blood samples to a lab, after which this data generates a dosage using a Bayesian Model-Informed Precision Dosing (MIPD) model generator, which can be in the cloud, and then transmitting this information to a health care provider, as discussed in further detail, below.

FIG. 4A schematically illustrates an exemplary kit as provided herein comprising syringes for administering a drug or drug combination in liquid form, and a hand-held volumetric dried blood spot or volumetric absorptive microsampling (VAMS) collection device; and

FIG. 4B schematically illustrates components of an exemplary kit, including a hand-held volumetric dried blood spot or volumetric absorptive microsampling (VAMS) collection device and syringes for administering a drug or drug combination in liquid form having a time stamp for recording use; including showing the patient's individualized dosing data as shown on a mobile phone (but can be any device, including a computer).

FIG. 5A FIG. 4B schematically illustrates components of an exemplary kit, including a hand-held volumetric dried blood spot or volumetric absorptive microsampling (VAMS) collection device and dosage units (which can be capsules, pills, tablets, geltabs and the like); and

FIG. 5B schematically illustrates how dosage units (which can be capsules, pills, tablets, geltabs and the like) in the kit are time stamped (the time the dosage unit is taken by the patient is recorded).

FIG. 6 illustrates TSC model simulations of dosing algorithms of 10,000 virtual patients, pre algorithm steady state, including model parameters from a population pharmacokinetic (PK) model, termed “TSC-MODEL”, which was devised based on a previously validated and published PK model and established using a Three-arm, Randomized, Double-blind, Placebo-controlled Study of the Efficacy and Safety of Two Trough-ranges of Everolimus as Adjunctive Therapy in Patients With Tuberous Sclerosis Complex (TSC) Who Have Refractory Partial-onset Seizures (Phase III)-coordination clinical study data, as discussed in further detail, below.

FIG. 7 graphically illustrates the time course of an exemplary patient's measurements in a single period, versus 4 period fluctuations, as discussed in further detail, below.

FIG. 8 graphically illustrates the time course of an exemplary patient's measurements in a single period, versus 14 period fluctuations, as discussed in further detail, below.

FIG. 9 graphically illustrates the 95%-confidence interval of reaching a target trough blood drug concentration using a Monte Carlo simulation of test dose method applied to 10000 samples, as discussed in further detail, below.

FIG. 10 graphically illustrates predicted versus target trough blood drug concentration using Test Dose method to determine starting and loading doses, as discussed in further detail, below.

Like reference symbols in the various drawings indicate like elements.

### DETAILED DESCRIPTION

In alternative embodiments, provided are therapeutic drug compositions, including products of manufacture and kits, and methods, for using them. In alternative embodiments, provided are novel methods for efficacious and safe dosaging of the therapeutic drug compositions. For example, methods for efficacious and safe-dosaging are used to address dosaging problems when administering sirolimus (also known as rapamycin), which has a narrow therapeutic index, high interpatient and within patient pharmacokinetic variability and non-optimized dosing, and if not dosaged properly sirolimus administration can lead to death or severe morbidity.

In alternative embodiments, the dosaging of therapeutic drug compositions as provided herein is determined using a Bayesian Model-Informed Precision Dosing (MIPD) model generator (which can be in a cloud-based program) (see for example: Darwich et al, Annu Rev Pharmacol Toxicol. Jan. 6, 2021; 61:225-245; Kantasiripitak et al Front. Pharmacol., 7 May 2020), which incorporates patient pharmacokinetics (PK)/dosing/timing data to create an individualized (for example, based on age and weight) MIPD model generator and dose recommendations or retesting parameters. In alternative embodiments, the dosage is first determined and then provided to the individual, for example, the health care practitioner, who either administers the drug or prescribes how the drug is to be taken by an individual in need thereof (for example, a patient), for example, for treatment of a rare disease as described herein.

In alternative embodiments, provided are artificial intelligence (AI)-enabled therapeutic drug compositions (bMEDs™, orbbMEDs ADVANCED™), including bMEDs™, orbbMEDs ADVANCED™ amifampridine for Lambert Eaton Myasthenic Syndrome (LEMS), bMEDs™, orbMEDs ADVANCED™ deflazacort to Duchenne Muscular Dystrophy (DMD), bMEDs™, orbMEDs ADVANCED™ propranolol for Infantile Hemangioma, bMEDs™, orbbMEDs ADVANCED™ serabelisib for PIK3CA-Related Overgrowth Spectrum (PROS), bMEDs™, orbMEDs ADVANCED™ sirolimus for Kaposiform Hemangioendothelioma with Kasabach-Merritt Phenomenon (“KHE w/KMP”), bMED™ everolimus for TSC-associated epilepsy, TSC-associated SEGA, TSC-associated renal angiomyolipoma, breast cancer, pNET, renal cancer, or allograph organ rejection prophylaxis, and bMEDs™ orbbMEDs ADVANCED™ for rare diseases and other diseases as provided herein.

In alternative embodiments, artificial intelligence (AI)-enabled therapeutic drug compositions (bMEDs ADVANCED™) as provided herein comprise individualized doses of a Narrow Therapeutic Index (“NTI”) drug or drugs and AI-enabled companion diagnostics for individualized PK-guided dosing. Hence bMEDs ADVANCED™ as provided herein enable patients to benefit from NTI drugs with optimally safe and effective drug concentrations and without the risks associated with high PK variability. In alternative embodiments, artificial intelligence (AI)-enabled therapeutic drug dosages are provided to a health care practitioner and/or to a patient for, optionally, the treatment of a disease or condition as described herein, for example, for treatment of a rare disease as described herein.

In alternative embodiment, bMEDs ADVANCED™ amifampridine comprises a combination product kit comprised of individualized doses of amifampridine (for example, from between about 5 mg to 200 mg) and an AI-enabled companion diagnostic for individualized PK-guided dosing. In alternative embodiments, individualized PK-guided dosing amounts are provided to a health care practitioner and/or to a patient for, optionally, the treatment of a disease or condition as described herein, for example, for treatment of a rare disease as described herein.

In alternative embodiment, bMEDs ADVANCED™ propranolol comprises a combination product kit comprised of individualized doses of propranolol (for example, from between about 0.5 mg to 50 mg) and an AI-enabled companion diagnostic for individualized PK-guided dosing. In alternative embodiments, individualized PK-guided dosing amounts are provided to a health care practitioner and/or to a patient for, optionally, the treatment of a disease or condition as described herein, for example, for treatment of a rare disease as described herein.

In alternative embodiment bMEDs ADVANCED™ deflazacort comprises a combination product kit comprised of individualized doses of deflazacort (for example, from between about 2 mg to 200 mg) and AI-enabled companion diagnostic for individualized PK-guided dosing. In alternative embodiments, individualized PK-guided dosing amounts are provided to a health care practitioner and/or to a patient for, optionally, the treatment of a disease or condition as described herein, for example, for treatment of a rare disease as described herein.

In alternative embodiment bMEDs ADVANCED™ ataluren comprises a combination product kit comprised of individualized doses of ataluren (for example, from between about 25 mg to 4 grams) and AI-enabled companion diagnostic for individualized PK-guided dosing. In alternative embodiments, individualized PK-guided dosing amounts are provided to a health care practitioner and/or to a patient for, optionally, the treatment of a disease or condition as described herein, for example, for treatment of a rare disease as described herein.

In alternative embodiment, bMEDs ADVANCED™ sirolimus comprises a combination product kit comprised of individualized doses of sirolimus (for example, from between about 0.1 mg to 10 mg) and AI-enabled companion diagnostic for individualized PK-guided dosing. In alternative embodiments, individualized PK-guided dosing amounts are provided to a health care practitioner and/or to a patient for, optionally, the treatment of a disease or condition as described herein, for example, for treatment of a rare disease as described herein.

In alternative embodiments, the dosaging of therapeutic drug compositions as provided herein is determined using a Therapeutic Drug Monitoring (TDM) Software as a Service (SaaS) dosing tool (which can be in a cloud-based program), which incorporates actual dose amounts and dose administration timing, actual blood drug concentrations blood drug and blood collection timing and the target drug concentration to create an individualized dose recommendations and retesting parameters. In alternative embodiments, dosing amounts determined using a Therapeutic Drug Monitoring (TDM) Software are provided to a health care practitioner and/or to a patient for, optionally, the treatment of a disease or condition as described herein, for example, for treatment of a rare disease as described herein.

In alternative embodiments, provided are artificial intelligence (AI)-enabled therapeutic drug compositions (bMEDs BASIC™), including bMEDs BASIC™ amifampridine for Lambert Eaton Myasthenic Syndrome (LEMS), bMEDs BASIC™ deflazacort to Duchenne Muscular Dystrophy (DMD), bMEDs BASIC™ ataluren to Duchenne Muscular Dystrophy (DMD), bMEDs BASIC™ propranolol for Infantile Hemangioma, bMEDsBASIC™ serabelisib for PIK3CA-Related Overgrowth Spectrum (PROS), bMEDsBASIC™ sirolimus (also known as rapamycin) for Kaposiform Hemangioendothelioma with Kasabach-Merritt Phenomenon (“KHE w/KMP”), and bMEDs BASIC™ for rare diseases as provided herein.

In alternative embodiments, artificial intelligence (AI)-enabled therapeutic drug compositions (bMED™ or bMEDs ADVANCED™) as provided herein comprise individualized doses of a Narrow Therapeutic Index (“NTI”) drug(s) (Narrow therapeutic index (NTI) drugs are drugs where small differences in dose or blood concentration may lead to serious therapeutic failures and/or adverse drug reactions that are life-threatening or result in persistent or significant disability or incapacity) and AI-enabled companion diagnostics for individualized PK-guided dosing. Hence bMED™ or bMEDs ADVANCED™ as provided herein enable patients to benefit from NTI drugs with optimally safe and effective drug concentrations and without the risks associated with high PK variability.

In alternative embodiment, bMED™ or bMEDs ADVANCED™ amifampridine comprises a combination product kit comprised of individualized doses of amifampridine (for example, from between about 5 mg to 200 mg) and an AI-enabled companion diagnostic for individualized PK-guided dosing.

In alternative embodiment, bMED™ or bMEDs ADVANCED™ propranolol comprises a combination product kit comprised of individualized doses of propranolol (for example, from between about 0.5 mg to 50 mg) and an AI-enabled companion diagnostic for individualized PK-guided dosing.

In alternative embodiment bMED™ or bMEDs ADVANCED™ deflazacort comprises a combination product kit comprised of individualized doses of deflazacort (for example, from between about 2 mg to 200 mg) and AI-enabled companion diagnostic for individualized PK-guided dosing.

In alternative embodiment bMED™ or bMEDs ADVANCED™ ataluren comprises a combination product kit comprised of individualized doses of ataluren (for example, from between about 25 mg to 4 grams) and AI-enabled companion diagnostic for individualized PK-guided dosing.

In alternative embodiment, bMED™ or bMEDs ADVANCED™ sirolimus comprises a combination product kit comprised of individualized doses of sirolimus (for example, from between about 0.1 mg to 10 mg) and AI-enabled companion diagnostic for individualized PK-guided dosing.

In alternative embodiment, bMED™ or bMEDs ADVANCED™ everolimus comprises a combination product kit comprised of individualized doses of everolimus (for example, from between about 0.1 mg to 40 mg) and AI-enabled companion diagnostic for individualized PK-guided dosing.

In alternative embodiments, the dosaging of therapeutic drug compositions as provided herein is determined using a Therapeutic Drug Monitoring (TDM) Software as a Service (SaaS) dosing tool (which can be in a cloud-based program), which incorporates actual dose amounts and dose administration timing, actual blood drug concentrations blood drug and blood collection timing and the target drug concentration to create an individualized dose recommendations and retesting parameters.

In alternative embodiments, provided are artificial intelligence (AI)-enabled therapeutic drug compositions (bMED™ or bMEDs BASIC™), including bMED™ or bMEDs BASIC™ amifampridine for Lambert Eaton Myasthenic Syndrome (LEMS), bMED™ or bMEDs BASIC™ deflazacort to Duchenne Muscular Dystrophy (DMD), bMED™ or bMEDs BASIC™ ataluren to Duchenne Muscular Dystrophy (DMD), bMED™ or bMEDs BASIC™ propranolol for Infantile Hemangioma, bMED™ or bMEDs BASIC™ serabelisib for PIK3CA-Related Overgrowth Spectrum (PROS), bMED™ or bMEDs BASIC™ sirolimus (also known as rapamycin) for Kaposiform Hemangioendothelioma with Kasabach-Merritt Phenomenon (“KHE w/KMP”), bMED™ everolimus for TSC-associated epilepsy, TSC-associated SEGA, TSC-associated renal angiomyolipoma, breast cancer, pNET, renal cancer, or allograph organ rejection prophylaxis, and bMED™ or bMEDs BASIC™ for rare diseases as provided herein.

In alternative embodiments, artificial intelligence (AI)-enabled therapeutic drug compositions (bMED™ or bMEDs BASIC™) as provided herein comprise individualized doses of a Narrow Therapeutic Index (“NTI”) drug(s) and AI-enabled companion diagnostics for individualized blood drug concentration dosing. Hence, bMED™ or bMEDs Basics as provided herein enable patients to benefit from NTI drugs with optimally safe and effective drug concentrations and without the risks associated with high PK variability.

In alternative embodiments, bMED™ or bMEDs BASIC™ amifampridine comprises a combination product kit comprised of individualized doses of amifampridine (for example, from between about 5 mg to 200 mg) and an AI-enabled companion diagnostic for individualized blood drug concentration dosing. In alternative embodiments the dosage of the amifampridine is determined by a method as provided herein and is provided to a health care practitioner or to the patient.

In alternative embodiment, bMED™ or bMEDs BASIC™ propranolol comprises a combination product kit comprised of individualized doses of propranolol (for example, from between about 0.5 mg to 50 mg) and an AI-enabled companion diagnostic for individualized blood drug concentration dosing. In alternative embodiments the dosage of the propranolol is determined by a method as provided herein and is provided to a health care practitioner or to the patient.

In alternative embodiment, bMED™ or bMEDs BASIC™ deflazacort comprises a combination product kit comprised of individualized doses of deflazacort (for example, from between about 2 mg to 200 mg) and AI-enabled companion diagnostic for individualized blood drug concentration dosing. In alternative embodiments the dosage of the deflazacort is determined by a method as provided herein and is provided to a health care practitioner or to the patient.

In alternative embodiment, bMED™ or bMEDs BASIC™ ataluren comprises a combination product kit comprised of individualized doses of ataluren (for example, from between about 25 mg to 4 grams) and AI-enabled companion diagnostic for individualized blood drug concentration dosing. In alternative embodiments the dosage of the ataluren is determined by a method as provided herein and is provided to a health care practitioner or to the patient.

In alternative embodiment, bMED™ or bMEDs BASIC™ sirolimus (also known as rapamycin) comprises a combination product kit comprised of individualized doses of sirolimus (for example, from between about 0.1 mg to 10 mg) and AI-enabled companion diagnostic for individualized blood drug concentration dosing. In alternative embodiments the dosage of the sirolimus is determined by a method as provided herein and is provided to a health care practitioner or to the patient.

In alternative embodiment, bMED™ or or bMEDs BASIC™ everolimus comprises a combination product kit comprised of individualized doses of everolimus (for example, from between about 0.1 mg to 40 mg or 80 mg) and AI-enabled companion diagnostic for individualized blood drug concentration dosing. In alternative embodiments the dosage of the everolimus is determined by a method as provided herein and is provided to a health care practitioner or to the patient.

### Calculation of Drug Amount to Reach Drug Plasma/Blood Target

In alternative embodiments, dosaging (or dosage amounts, or scheduling) is/are first determined then provided to the individual who will be administering the drug or drugs, for example, a health care provider such as a physician, or nurse, or can be prescribed by the health care provider and self-administered by a user or patient.

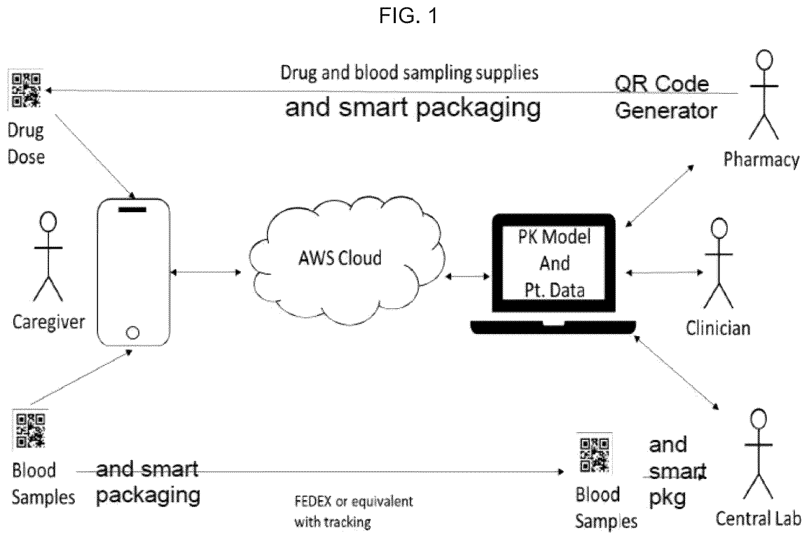

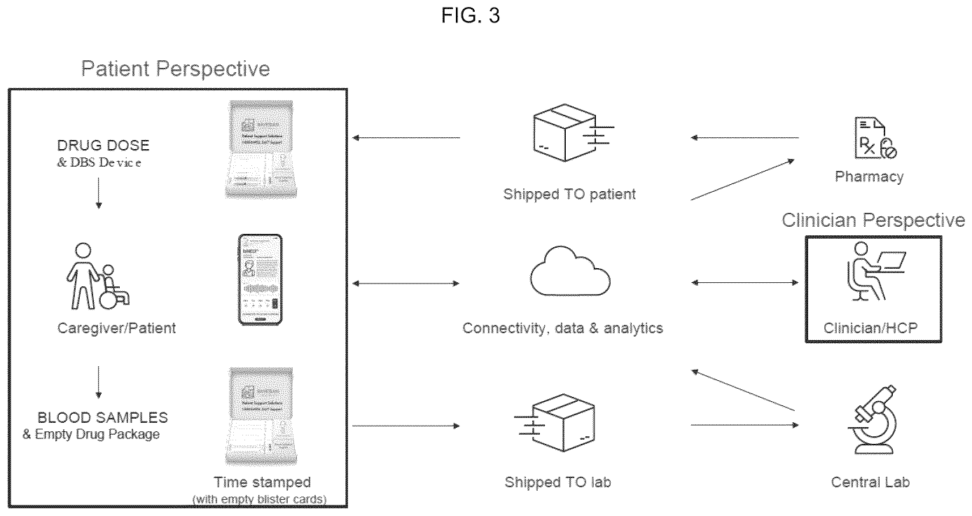

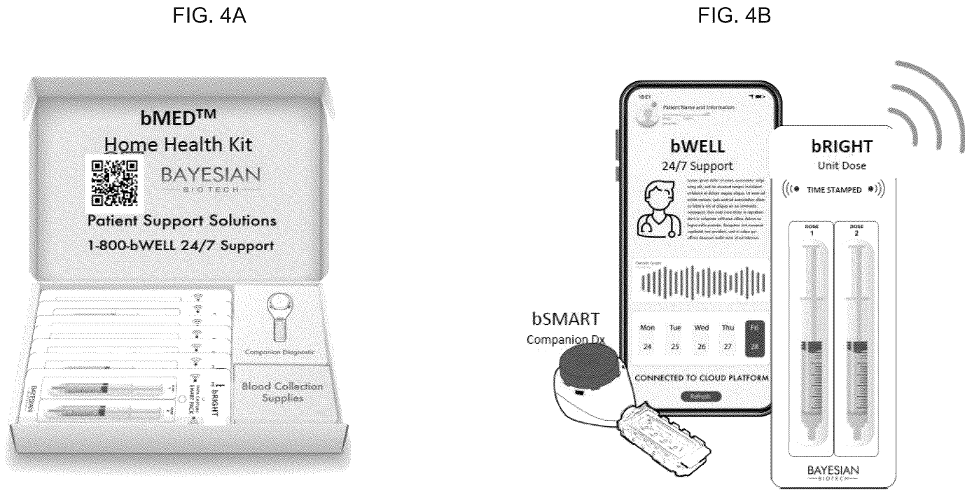

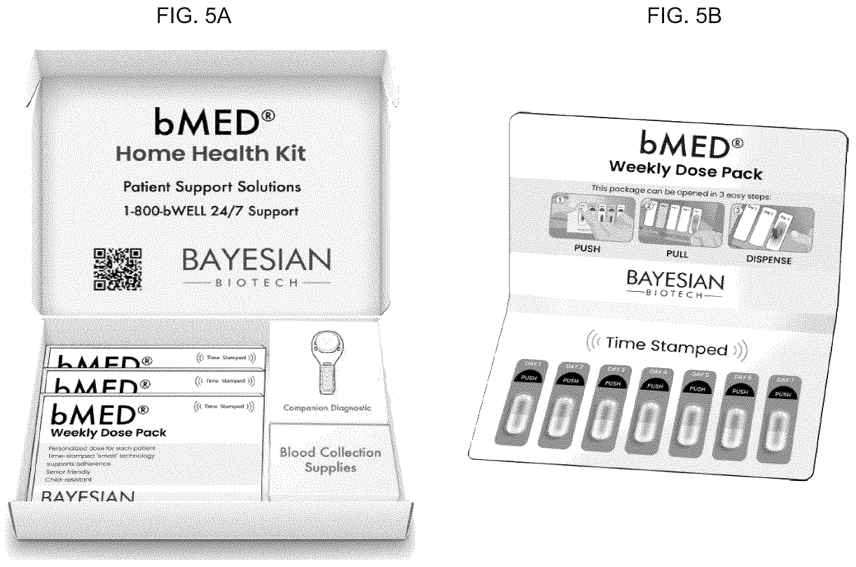

In alternative embodiments, to determine dosaging of therapeutic drug compositions as provided herein a dose calculator or a Bayesian Model Informed Precision Dosing (MIPD) is used with Dried Blood Spot (DBS), a volumetric dried blood spot, volumetric absorptive microsampling (VAMS), or an at-home high-volume liquid blood, sample acquisition kit or device (also called the “companion diagnostic”) and optionally the volumetric dried blood spot, volumetric absorptive microsampling (VAMS) or the at-home high-volume liquid blood collection device comprises a MITRA™ device (by NEOTERYX™), or a bladeless microneedle array as in a TAPMICRO™ or HALO™ device (yourbio, Medford, MA), (see for example, Baillargeon et al., ACS Measurement Science Au (2022) vol 2(1):31-38; Tuaillon et al., Front. Microbiol. 9 Mar. 2020); and the blood sample is sent to a central lab for analysis of the DBS or liquid blood sample to measure and assess drug blood (or plasma) concentration, wherein optionally liquid chromatography-mass spectrometry (LC-MS), or High-performance liquid chromatography (HPLC)-mass spectrometry (MS) (HPLC-MS) is used to determine the amount of drug in the DBS or liquid blood sample and thereby allow determination of drug blood or plasma concentration (and thus allowing for adjustments in drug dosaging, as appropriate—where the dose calculator or MIPD is used to determine optimal dosaging), and smart packaging, where each individual patient and each DBS, a volumetric dried blood spot, volumetric absorptive microsampling (VAMS), or an at-home high-volume liquid blood companion diagnostic and each drug storage pocket, packette or storage container (for example, in a sachet or a blister pack or clamshell) has a unique QR code (an initialism for quick response code) (see for example, FIG. 1, FIG. 3);

In any of the methods disclosed herein, the blood samples can be collected before administration of the drug compound or drug combination and at multiple time points, e.g., at about 5 minutes, about 15 minutes, about 30 minutes, about 45 minutes, about 60 minutes, about 2 hours, about 2.5 hours, about 4 hours, about 6 hours, about 8 hours or about 12 hours or about 24 hours, or anytime between about 1 minute and 12 to 24 hours or 1 minute to 96 hours. Alternatively, the blood samples can be collected at about 0.08 hour, 0.5±0.1 hour, 1.5±0.3 hours, 2±0.4 hours, 4±1 hours, 8±1.5 hours or 12±2 hours, 24±2 hours, 36±2 hours, 48 hours±2 hours, 72 hours±2 hours, or 96 hours±2 hours after the drug compound is administered. In another example, the blood samples can be collected between about: 0.08 to 0.1.9 hour; 0.33 to 0.90 hour; 1.3 to 2.7 hours; 3.6 to 4.0 hours; 7.0 to 9.0 hours; 10 to 12 hours; 20 to 24 hours; 32 to 40 hours; 44 to 52 hours; 52 to 68 hours; 68 to 76 hours, 76 hours to 92 hours, or 92 to 100 hours, after the administration of the drug compound;
and in alternative embodiments one, several or all of the following are used:
a. For starting dose, use lowest effective dose or a test dose;
b. Use individualized dose calculator or Model Model-Informed Precision Dosing (MIPD) model: recommends either a new dose (if actual blood concentrations are consistent with model estimates), additional tests and/or query of sites if actual blood concentrations are inconsistent with model estimates;
c. Draw and analyze multiple (for example 1 to 10) blood samples, for example, a volumetric dried blood spot, volumetric absorptive microsampling (VAMS), dried blood spot blood or at-home high-volume liquid blood draws per month, for example, 1 to 4 dried blood spot blood or at-home high-volume liquid blood draws per month, or 1 to 3 blood draws per month;
d. Use of centralized lab and analysis samples using for example HPLC MS/MS;
e. Use of smart packaging to time stamp use of the drug product and the dried blood spot (DBS), volumetric dried blood spot, volumetric absorptive microsampling (VAMS), or at-home high-volume liquid blood companion diagnostic device and to identify the unique patient and dose amounts, and where the time stamp verifies the patient is taking or is given the correct amounts of drug(s) at the right time(s), and optionally the “companion diagnostic” is a dried blood collection device, such as a TAP® or TAP II® Push-button blood collection device (Tasso Inc., WA, USA), or an M20® push button blood collection device (Tasso Inc., WA, USA), ONEDRAW™ blood collection device (Thorne Health Tech, Inc. SC, USA), or a MITRA® Specimen Collection Kit (Neoteryx Inc., CA, USA), or a TASSOONE™ Plus or at-home high-volume liquid blood collection device (Tasso Inc., WA, USA), that are sent to and analyzed by a central lab using for example HPLC MS/MS;
f. Read the time stamp at the central lab and incorporated into the dose calculator or MIPD model;
g. and optionally add with infant/child body weight growth trajectory and clearance trajectory to individualized MIPD model as described in for example: Emoto et al, Characterizing the Developmental Trajectory of Sirolimus Clearance in Neonates and Infants, CPT Pharmacometrics Syst. Pharmacol. (2016) 5, 411-417; Emoto et al, Ther. Drug Monit. (2016) Risk Assessment of Drug-Drug Interactions of Calcineurin Inhibitors Affecting Sirolimus Pharmacokinetics in Renal Transplant Patients, Oct; 38(5):607-13; Mizuno, Emoto, et al., Model-based precision dosing of sirolimus in pediatric patients with vascular anomalies, Eur J Pharm Sci. (2017) 109:S124-31; Polasek et al., Clinical Pharmacol. Drug Develop. (2019) vol 8(4):418-425).

This means the dose amount will change monthly based on change in the moving average (or weighted average) of the current dose/actual trough or AUC blood (or plasma) drug concentration and prior doses/trough or AUC blood (or plasma) drug concentrations, or optionally, based on changes in blood (or plasma) drug concentration and the developmental trajectory of the Vd, the allometric scaling of clearance and the maturation of P450 enzymes.

In alternative embodiments, an exemplary allocation of systems is used as illustrated in FIG. 1, which illustrates that each individual patient, each DBS (dried blood sample), volumetric dried blood spot or volumetric absorptive microsampling (VAMS) or at-home high-volume liquid blood, companion diagnostic and each drug storage pocket, packette or storage container (for example, in a sachet or a blister pack or clamshell) has a unique QR code (an initialism for quick response code) (generated by a QR code generator) to generate a “smart packaging”, where the patient or care giver (for example, a technician, nurse or physician), after administering all the drugs in a kit or package (such as a sachet or a blister pack or clamshell) sends both the used (drug depleted) kit or package (which has been time-stamped as of the opening of each individual drug container in the sachet or a kit or package) and all the blood-sampled DBSs (dried blood samples, the companion diagnostic), volumetric dried blood spot or volumetric absorptive microsampling (VAMS) or at-home high-volume liquid blood, send to a central facility (for example, a lab) for reading and analysis, where the central lab utilizing the time stamp can verify the times each individual drug was accessed and administered to the patient. In FIG. 1 use of a cell phone by the caregiver is optional, and any cloud service can be used. The data generated and collated (for example, the times recorded by the time stamps) by the central lab is transmitted to the patient, caregiver, technician, nurse and/or physician, and optionally to a central database.

In alternative embodiments, a drug monitoring system as used in methods as provided herein comprises a therapeutic drug or plasma protein dosing regimen apparatus and a drug monitoring tool. In alternative embodiments, the therapeutic drug or plasma protein dosing regimen apparatus comprises a model generator configured to create a Bayesian model of pharmacokinetic (PK) profiles of sampled patients. In alternative embodiments, the Bayesian model comprises:
(i) a therapeutic drug clearance; and
(ii) a volume of distribution relationship for a therapeutic drug based upon at least one of patient age or body weight.

In alternative embodiments, the therapeutic drug dosing regimen apparatus also comprises a PK server, which can be configured to determine an approximate PK profile of a patient based upon the Bayesian model, a half-life of the therapeutic drug within the patient, and at least one of an age of the patient or a weight of the patient. The PK server can be configured to determine the therapeutic drug dosing regimen including a dosage and a therapeutic plasma or blood drug level over a time period based upon the approximate PK profile of the patient, modify the therapeutic drug dosing regimen in response to receiving a dosage to the patient, and/or transmit the modified therapeutic drug dosing regimen to the client device.

In alternative embodiments, the drug monitoring tool comprises a data receiver configured to receive the pharmacokinetic (PK) profile of a patient. In addition, the drug monitoring tool can comprise an interactive user interface configured to display to the patient a time-varying therapeutic drug level of the patient. The time-varying therapeutic drug level can be based on an administered dose of a therapeutic drug and the PK profile of the patient.

In alternative embodiments, the PK server is configured to create patient-specific models using the pharmacokinetic model provided by the model generator to account for the patient-specific pharmacokinetic variance. In this manner, one or more base models are refined or adjusted by the PK server responsive to receiving previous treatment information for a specific patient. The PK server may be configured to store the patient-specific model to the database for subsequent uses by the same healthcare provider or other healthcare providers.

In alternative embodiments, once a PK profile for a patient is generated, the PK server is configured to transmit the PK profile to the drug monitoring tools (physician and caregiver devices). In some embodiments, the PK server can encrypt the data file prior to transmission. The encryption can be specific to a particular patient such that the drug monitoring tool can only open and process a received PK profile if the tool has a patient specific authentication key.

In alternative embodiments, the environment can include an ecosystem monitoring system that is coupled to the network and in communication with both the remote server and drug monitoring tools (physician and caregiver devices). The system can provide notification to a pharmacist to prepare the particular therapeutic drug for purchase by a patient. For example, the system can determine that the patient has a threshold amount of the drug left such that the patient will be in need of the drug in the near future. Similarly, the system can contact a physician to ensure that physician has real-time information associated with the patient. Thus, the physician can take immediate actions to care for the patient if a need arises.

FIG. 1 is a flow diagram of an exemplary method for generating a moving average of administered drug blood concentrations or a PK profile of a particular patient in accordance with an example embodiment of the present disclosure. The method includes collecting patient information, including patient sample, medical history, etc. The exemplary method includes generating a moving average of administered drug blood concentrations or a PK profile for a patient. The PK profile can be generated as described in Mizuno, Emoto, et al., Model-based precision dosing of sirolimus in pediatric patients with vascular anomalies, Eur J Pharm Sci. (2017) 109:S124-31.

In alternative embodiments, methods as provided herein comprise encoding the PK profile. In alternative embodiments, the PK profile can be encoded by the PK Server using known or yet to be known electronic data encoding techniques. In alternative embodiments, the method includes transmitting the PK profile to a drug monitoring tools.

In alternative embodiments, to determine dosaging of a therapeutic drug composition or combination as provided herein a dose calculator or Therapeutic Drug Monitoring (TDM) is used with Dried Blood Spot (DBS) or at-home high-volume liquid blood sample acquisition (the “companion diagnostic”) (see for example, Baillargeon et al., ACS Measurement Science Au (2022) vol 2(1):31-38; Tuaillon et al., Front. Microbiol. 9 Mar. 2020), followed by analysis of the DBS or liquid blood sample to measure and assess drug blood (or plasma) concentration, wherein optionally liquid chromatography-mass spectrometry (LC-MS), or High-performance liquid chromatography (HPLC)-mass spectrometry (MS) (HPLC-MS) is used to determine the amount of drug in the DBS or liquid blood sample and thereby allow determination of drug blood or plasma concentration (and thus allowing for adjustments in drug dosaging, as appropriate, where optionally the dose calculator or TDM is used to determine optimal dosaging), and smart packaging, where each individual patient and each DBS or at-home high-volume liquid blood companion diagnostic and each drug storage pocket, packette or storage container (for example, in a sachet or a blister pack or clamshell) has a unique QR code (an initialism for quick response code) (see for example, FIG. 1);
and in alternative embodiments one, several or all of the following are used:
   a. For starting dose, use lowest effective dose or a test dose;
   b. Use individualized dose calculator or Therapeutic Drug Monitoring (TDM) software: recommends a new dose (if the calculated adjusted dose is different than the current dose and there is evidence of no missed doses or no temporary changes in the patient health or other status), additional tests and/or query of patient if there insufficient evidence that the change in calculated adjusted dose were associated with permanent changes in the patient health and other status;
   c. Draw and analyze 1 to 6 dried blood spot, volumetric dried blood spot, volumetric absorptive microsampling (VAMS) or the at-home high-volume liquid blood collection device blood draws per month, or 2 to 4 or 3 blood draws per month;
   d. Use of centralized lab and analysis samples using for example HPLC MS/MS;
   e. Instruct patient to write the time of the use of the drug product and the dried blood spot (DBS), volumetric dried blood spot, volumetric absorptive microsampling (VAMS) or at-home high-volume liquid blood companion diagnostic device,
   the “companion diagnostic” is a volumetric dried blood spot, volumetric absorptive microsampling (VAMS), or a dried blood collection device, such as a TAP® or TAP II©Push-button blood collection device (Tasso Inc., WA, USA), or an M20® push button blood collection device (Tasso Inc., WA, USA), ONEDRAWBlood Collection Device (Thorne Health Tech, Inc. SC, USA), or a MITRA® Specimen Collection Kit (Neoteryx Inc., CA, USA), that are sent to and analyzed by a central lab using for example HPLC MS/MS;
   f. Read the time of the use of the drug product and the dried blood spot (DB), volumetric dried blood spot, or volumetric absorptive microsampling (VAMS) companion diagnostic device, at the central lab and incorporated into the TDM calculation; and/or
   g. The dose calculator or Therapeutic Drug Monitoring (TDM) Software as a Service (SaaS) dosing tool provided a dose estimated based on new dose equal to the closest available dose amount to an adjusted dose which is equal to moving average of administered drug blood concentrations or the actual dose*target blood (or plasma) drug concentration/measured blood (or plasma) drug concentration.

In alternative embodiments, the blood (or plasma) drug concentration can be a trough concentration prior to the next dose administration, a drug concentration after a specified number of hours following the administration of a dose or an Area Under the Curve (AUC) concentration or for an AUC concentration for specified number of hours following the administration of a dose.

In alternative embodiments, an exemplary allocation of systems is used as illustrated in FIG. 3, which illustrates that each individual patient, each DBS (dried blood sample), volumetric dried blood spot, volumetric absorptive microsampling (VAMS), or a dried blood collection device (or companion diagnostic) and each drug storage pocket, packette or storage container (for example, in a sachet or a blister pack or clamshell) has a unique QR code (an initialism for quick response code) (generated by a QR code generator) to generate a “smart packaging”, where the patient or care giver (for example, a technician, nurse or physician), after administering all the drugs in a kit or package (such as a sachet or a blister pack or clamshell) sends both the used (drug depleted) kit or package (which has been time-stamped as of the opening of each individual drug container in the kit or package) and all the blood-sampled DBSs (dried blood samples, volumetric dried blood spot, volumetric absorptive microsampling (VAMS), or a dried blood collection device, also called the companion diagnostic) to a central facility (for example, a lab) for reading and analysis, where the central lab utilizing the time stamp can verify the times each individual drug was accessed and administered to the patient. In FIG. 1 use of a cell phone by the caregiver is optional, and any cloud service can be used. The data generated and collated (for example, the times recorded by the time stamps) by the central lab is transmitted to the patient, caregiver, technician, nurse and/or physician, and optionally to a central database.

In alternative embodiments, a drug monitoring system as used in methods as provided herein comprises a therapeutic drug or plasma protein dosing regimen apparatus and a drug monitoring tool. In alternative embodiments, the therapeutic drug or plasma protein dosing regimen apparatus comprises dose calculator that provides a recommendation for a dose based on:
(i) Current drug amount
(ii) Timing of the dose administration
(iii) The blood (or plasma) drug concentrations,
(iv) The timing of the blood sampling,
(v) The target blood (or plasma) drug concentration

In alternative embodiments, for dosing with a trough (or AUC) target blood (or plasma) drug concentration, the dose calculator will calculate an adjusted dose amount=current dose amount*target trough (or AUC) blood (or plasma) concentration/actual trough (or AUC) blood (or plasma) drug concentration. There-after, the dose calculator will determine the closest available dose amount to the adjusted dose amount and recommend the available dose amount. In alternative embodiments, the dose calculator will calculate an adjusted dose amount=target trough (or AUC) blood (or plasma) concentration*moving average of the current dose/actual plasma drug concentration and the previous one, two or three or up to twelve months' dose amount/blood (or plasma) drug concentration, or the adjusted dose amount=current dose amount*target trough (or AUC) blood (or plasma) concentration/moving average of the actual trough (or AUC) blood (or plasma) drug concentration of the current and the one, two, or three or up to twelve previous weeks (or months) with constant dose amount. There-after, the dose calculator will determine the closest available dose amount to the adjusted dose amount and recommend the available dose amount.

In alternative embodiments the dose calculator will calculate an adjusted dose amount equal to the target trough (or AUC) blood (or plasma) concentration*moving average of the current dose amount/actual trough (or AUC) blood (or plasma) drug concentration and the previous one, two or three or up to twelve months' dose amounts/trough (or AUC) blood (or plasma) drug concentrations, or the adjusted dose amount=current dose amount*target trough (or AUC) blood (or plasma) concentration/moving average of the actual trough (or AUC) blood (or plasma) drug concentration of the current and one, two, three or four or up to twelve previous weeks (or months) with constant dose amount, excluding the prior trough (or AUC) drug concentration measurements associated with a transient change in blood (or plasma) drug concentrations such as from a missed dose administration or a febrile illness, or before a persistent change in the dose amount/trough or AUC blood (or plasma) drug concentration such as from a drug-drug interaction.

In alternative embodiments, for dosing with an Area Under the Curve (AUC) target blood (or plasma) drug concentration for defined period of hours, the dose calculator will calculate an adjusted dose amount using the linear or logarithmic trapezoidal rule. Therefore the adjusted dose amount is equal to the current dose*AUC target blood (or plasma) drug concentration/actual AUC blood (or plasma) drug concentration using the linear or logarithmic trapezoidal rule for the defined period of time. There-after, the dose calculator will determine the closest available dose amount to the adjusted dose amount and recommend the available dose amount.

In alternative embodiments, the dose calculator will calculate an adjusted dose equal to the target AUC blood (or plasma) concentration*moving average of the current dose amount/actual AUC blood (or plasma) drug concentration and the previous one, two or three or up to twelve months' (or weeks') dose amounts/AUC blood (or plasma) drug concentration, or an adjusted dose amount equal to the current dose*AUC target blood (or plasma) drug concentration/moving average of the actual AUC blood (or plasma) drug concentration of the current and the one, two, or three previous weeks (or months) with constant dose amount. Alternatively, the dose calculator will calculate an adjusted dose equal to the current dose*AUC target blood (or plasma) drug concentration/moving average of the actual AUC blood (or plasma) drug concentration of the current and the one, two, or three or up to twelve previous weeks (or months) with constant dose amount, or an adjusted dose equal to the target AUC blood (or plasma) concentration*moving average of the current dose amount/actual AUC blood (or plasma) drug concentration and the previous one, two or three or up to twelve months' (or weeks') dose amounts/actual AUC blood (or plasma) drug concentration, excluding prior actual AUC drug concentration measurements associated with a transient change in blood (or plasma) drug concentrations, or before a persistent change in the dose amount/trough or AUC blood (or plasma) drug concentration.

For dosing with an AUC target of blood (or plasma) drug concentration to infinity, the dose calculator will first calculate the Actual AUC blood (or plasma) drug concentration using the linear and/or logarithmic trapezoidal rule and AUC-extrapolated. AUC extrapolated can be computed by taking the ratio of the drug concentration at the last drug concentration measurement and the terminal elimination rate constant. The terminal eliminate rate constant, k, must be calculated from the terminal elimination phase of the drug versus the time curve. The elimination rate constant, k, may be calculated using the formula (ln(C_last−1)−ln (C-last))/(T_last-T_last−1) or from the slope of the regression line of blood (or plasma) drug concentrations and times during the terminal elimination phase. Then the dose calculator will calculate the adjusted dose amount=current dose*AUC target blood (or plasma) drug concentration/actual AUC blood (or plasma) to infinity. There-after, the dose calculator will determine the closest available dose amount to the adjusted dose amount and recommend the available dose amount, which, optionally, is then reported to the patient and/or administered to the patient,

In alternative embodiments, the environment can include an ecosystem monitoring system that is coupled to the network and in communication with both the remote server and drug monitoring tools (physician and caregiver devices). The system can provide notification to a pharmacist to prepare the particular therapeutic drug for purchase by a patient. For example, the system can determine that the patient has a threshold amount of the drug left such that the patient will be in need of the drug in the near future. Similarly, the system can contact a physician to ensure that physician has real-time information associated with the patient. Thus, the physician can take immediate actions to care of the patient if a need arises.

FIG. 3 is a flow diagram of an exemplary method for generating a drug dose recommendation of a particular patient in accordance with an example embodiment of the present disclosure.

In alternative embodiments, methods as provided herein comprise encoding the patient and laboratory data. In alternative embodiments, the patient and laboratory data can be encoded by the Server using known or yet to be known electronic data encoding techniques. In alternative embodiments, the method includes transmitting the patient and laboratory data to a drug monitoring tools.

### Pharmaceutical Compositions and Formulations

In alternative embodiments, therapeutic combinations of drugs as provided herein, and drugs used to practice methods as provided herein, are formulated for administration by any or a variety of means including orally, parenterally, by inhalation spray, nasally, topically, intrathecally, intrathecally, intracerebrally, epidurally, intracranially or rectally. Therapeutic combinations of drugs as provided herein, and drugs used to practice methods as provided herein, can further comprise pharmaceutically acceptable carriers, adjuvants and vehicles. In alternative embodiments, therapeutic combinations of drugs as provided herein, and drugs used to practice methods as provided herein, are formulated for parenteral administration, including administration intrathecally, intracerebrally or epidurally (into a intrathecal, intracerebral, epidural space), subcutaneously, intravenously, intramuscularly and/or intraarterially; for example, by injection routes but also including a variety of infusion techniques. Intraarterial, intrathecal, intracranial, epidural, intravenous and other injections as used in some embodiments can include administration through catheters or pumps, for example, an intrathecal pump, or an implantable medical device (which can be an intrathecal pump or catheter).

In alternative embodiments, therapeutic combinations of drugs as provided herein, and drugs used to practice methods as provided herein, are formulated in accordance with a routine procedure(s) adapted for a desired administration route. In alternative embodiments, therapeutic combinations of drugs as provided herein, and drugs used to practice methods as provided herein, are formulated or manufactured as lyophilates, powders, granules, lozenges, liposomes, suspensions, solutions or emulsions in oily or aqueous vehicles, and can contain formulatory agents such as suspending, stabilizing and/or dispersing agents.

In alternative embodiments, therapeutic combinations of drugs as provided herein, and drugs used to practice methods as provided herein, can be formulated as a preparation for implantation or injection. Thus, for example, the compounds can be formulated with suitable polymeric or hydrophobic materials (for example, as an emulsion in an acceptable oil) or ion exchange resins, or as sparingly soluble derivatives (for example, as a sparingly soluble salt). Alternatively, the active ingredient can be in powder form for constitution with a suitable vehicle, for example, sterile pyrogen-free water, before use. Suitable alternative and exemplary formulations for each of these methods of administration can be found, for example, in Remington: The Science and Practice of Pharmacy, A. Gennaro, ed., 20th edition, Lippincott, Williams & Wilkins, Philadelphia, Pa.

In alternative embodiments, therapeutic combinations of drugs as provided herein, and drugs used to practice methods as provided herein, are formulated with sterile water or saline, a polyalkylene glycol such as a polyethylene glycol, an oil of synthetic or vegetable origin, a hydrogenated naphthalene and the like. In alternative embodiments, therapeutic combinations of drugs as provided herein, and drugs used to practice methods as provided herein, can be formulated in or with a biocompatible, biodegradable lactide polymer, a lactide/glycolide copolymer, or polyoxyethylene-polyoxypropylene copolymers can be useful excipients to control the release of active compounds.

In alternative embodiments, therapeutic combinations of drugs as provided herein, and drugs used to practice methods as provided herein, are administered using parenteral delivery systems such as ethylene-vinyl acetate copolymer particles, osmotic pumps, implantable infusion systems, intrathecal catheters, pumps and implants, and/or use of liposomes. Formulations for parenteral administration can also include glycocholate for buccal administration, methoxysalicylate for rectal administration, or citric acid for vaginal administration. Formulations for inhalation administration can contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally.

In alternative embodiments, therapeutic combinations of drugs as provided herein, and drugs used to practice methods as provided herein, are administered intranasally. When given by this route, examples of appropriate dosage forms are a nasal spray or dry powder, as is known to those skilled in the art. For example, a nasal formulation can comprise a conventional surfactant, generally a non-ionic surfactant. When a surfactant is employed in a nasal formulation, the amount present will vary depending on the particular surfactant chosen, the particular mode of administration (for example drop or spray) and the effect desired.

In alternative embodiments, therapeutic combinations of drugs as provided herein, and drugs used to practice methods as provided herein, are in the form of a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In alternative embodiments, sterile fixed oils are conventionally be employed as a solvent or suspending medium. For this purpose any bland fixed oil may be employed including synthetic mono- or diglycerides. In alternative embodiments, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule (ampoule) or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

In alternative embodiments, therapeutic combinations of drugs as provided herein, and drugs used to practice methods as provided herein, further comprise aqueous and non-aqueous sterile injection solutions that can contain (comprise) antioxidants, buffers, bacteriostats, bactericidal antibiotics and solutes that render the formulation isotonic with the bodily fluids of the intended recipient; and/or aqueous and non-aqueous sterile suspensions, which can include suspending agents and thickening agents.

In alternative embodiments, therapeutic combinations of drugs as provided herein, and drugs used to practice methods as provided herein, are formulated for topical administration, for example, in the form of a liquid, lotion, cream or gel. Topical administration can be accomplished by application directly on the treatment area. For example, such application can be accomplished by rubbing the formulation (such as a lotion or gel) onto the skin of the treatment area, or by a spray application of a liquid formulation onto the application or treatment area.

In alternative embodiments, therapeutic combinations of drugs as provided herein, and drugs used to practice methods as provided herein, comprise a bioimplant or a bioimplant material, and also can be coated with a compound of the invention or other compounds so as to improve interaction between cells and the implant.

In alternative embodiments, therapeutic combinations of drugs as provided herein, and drugs used to practice methods as provided herein, comprise minor amounts of wetting or emulsifying agents, or pH buffering agents.

In alternative embodiments, therapeutic combinations of drugs as provided herein, and drugs used to practice methods as provided herein, are formulated as a suppository, with traditional binders and carriers such as triglycerides.

In alternative embodiments, therapeutic combinations of drugs as provided herein, and drugs used to practice methods as provided herein, comprise oral formulations such as tablets, geltabs, capsules, pills, troches, lozenges (see, for example, as described in U.S. Pat. No. 5,780,055), aqueous or oil suspensions, dispersible powders or granules, emulsions, hard or soft capsules or geltabs, gels, jellies, syrups and/or elixirs. Compositions intended for oral use may be prepared according to any method known to the art for the manufacture of pharmaceutical compositions and such compositions may contain one or more agents including sweetening agents, taste-masking agents, flavoring agents, coloring agents and preserving agents, in order to provide a palatable preparation. Oral formulations can include standard carriers such as pharmaceutical grades of mannitol, lactose, starch, magnesium stearate, polyvinyl pyrrolidone, sodium saccharine, cellulose, magnesium carbonate, etc. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In alternative embodiments, formulations for oral use are hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

In alternative embodiments, therapeutic combinations of drugs as provided herein, and drugs used to practice methods as provided herein, comprise aqueous suspensions comprising the active material in admixture with excipients suitable for the manufacture of aqueous suspensions. Exemplary excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (for example, lecithin), a condensation product of an alkylene oxide with a fatty acid (for example, polyoxyethylene stearate), a condensation product of ethylene oxide with a long chain aliphatic alcohol (for example, heptadecaethyleneoxycetanol), a condensation product of ethylene oxide with a partial ester derived from a fatty acid and a hexitol anhydride (for example, polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

In alternative embodiments, therapeutic combinations of drugs as provided herein, and drugs used to practice methods as provided herein, comprise oil suspensions that can be formulated by suspending the active ingredient (for example, a compound of this invention) in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

In alternative embodiments, therapeutic combinations of drugs as provided herein, and drugs used to practice methods as provided herein, include an agent which controls release of the compound, thereby providing a timed or sustained release compound.

In alternative embodiments, therapeutic combinations of drugs as provided herein, and drugs used to practice methods as provided herein, are formulated or made as a multiparticulate and/or a solid dispersion formulation, for example, as described in, for example, U.S. Patent App. Pub. No. 20080118560, for example, comprising a hydrophobic matrix former which is a water-insoluble, non-swelling amphiphilic lipid; and a hydrophilic matrix former which is a meltable, water-soluble excipient. In one embodiment, therapeutic combinations of drugs as provided herein, and drugs used to practice methods as provided herein, are contained in sachets, tablets, pills, capsules, troches, and the like comprising any combination of a binder, for example, as a starch, polyvinyl pyrrolidone, gum tragacanth or gelatin; a filler, such as microcrystalline cellulose or lactose; a disintegrating agent, such as crospovidone, sodium starch glycolate, corn starch, and the like; a lubricant, such as magnesium stearate, stearic acid, glyceryl behenate; a glidant, such as colloidal silicon dioxide and talc; a sweetening agent, such as sucrose or saccharin, aspartame, acesulfame-K; and/or flavoring agent, such as peppermint, methyl salicylate, or orange flavoring. When the dosage unit form is a capsule, it also can comprise a liquid carrier, such as a fatty oil.

In alternative embodiments, therapeutic combinations of drugs as provided herein, and drugs used to practice methods as provided herein, comprise (or are contained or packaged in) unit dosage formulations having a coating, for example, a coat comprising a sugar, shellac, sustained and/or other enteric coating agents, or any pharmaceutically pure and/or nontoxic agents.

In alternative embodiments, therapeutic combinations of drugs as provided herein, and drugs used to practice methods as provided herein, comprise (or are contained or packaged in) unit dosage formulations, wherein each different compound of the composition or product of manufacture is contained in a different layer of a pill, tablet or capsule, for example, as described in U.S. Pat. No. 7,384,653, for example, having an outer base-soluble layer and an inner acid-soluble layer. In alternative embodiments, therapeutic combinations of drugs as provided herein, and drugs used to practice methods as provided herein, comprise (or are contained or packaged in) unit dosage formulations, wherein each different compound of the composition or product of manufacture is contained in a liquid or a gel of different viscosity, for example, described in U.S. Patent App. Pub. No. 20050214223. In alternative embodiments, therapeutic combinations of drugs as provided herein, and drugs used to practice methods as provided herein, comprise (or are contained or packaged in) unit dosage formulations having reduced abuse potential, for example, as described in U.S. Patent App. Pub. No. 20040228802, for example, comprising a bittering agent, a bright deterrent/indicator dye, or a fine insoluble particulate matter.

### Carriers

In alternative embodiments, therapeutic combinations of drugs as provided herein, and drugs used to practice methods as provided herein, comprise or are formulated with or as aqueous or non-aqueous solutions, suspensions, emulsions and solids. Examples of non-aqueous solvents suitable for use as disclosed herein include, but are not limited to, propylene glycol, polyethylene glycol, vegetable oils such as olive oil, and injectable organic esters such as ethyl oleate. In alternative embodiments, aqueous carriers can comprise water, ethanol, alcoholic/aqueous solutions, glycerol, emulsions and/or suspensions, including saline and buffered media. Oral carriers can be elixirs, syrups, capsules, tablets and the like.

In alternative embodiments, liquid carriers are used to manufacture or formulate therapeutic combinations of drugs as provided herein, and drugs used to practice methods as provided herein, including carriers for preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can comprise other suitable pharmaceutical additives such as solubilizers, emulsifiers, buffers, preservatives, sweeteners, flavoring agents, suspending agents, thickening agents, colors, viscosity regulators, stabilizers or osmo-regulators.

In alternative embodiments, liquid carriers used to manufacture or formulate compounds of this invention comprise water (partially containing additives as above, for example cellulose derivatives, alternatively sodium carboxymethyl cellulose solution), alcohols (including monohydric alcohols and polyhydric alcohols, for example glycols) and their derivatives, and oils (for example fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

In alternative embodiments, solid carriers are used to manufacture or formulate therapeutic combinations of drugs as provided herein, and drugs used to practice methods as provided herein, including solid carriers comprising substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (for example, povidone, gelatin, hydroxypropylmethyl cellulose), lubricant, inert diluent, preservative, disintegrant (for example, sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In alternative embodiments, parenteral carriers are used to manufacture or formulate therapeutic combinations of drugs as provided herein, and drugs used to practice methods as provided herein, including parenteral carriers suitable for use as disclosed herein include, but are not limited to, sodium chloride solution, Ringer's dextrose, dextrose and sodium chloride, lactated Ringer's and fixed oils. Intravenous carriers can comprise fluid and nutrient replenishers, electrolyte replenishers such as those based on Ringer's dextrose and the like. Preservatives and other additives can also comprise, for example, antimicrobials, antioxidants, chelating agents, inert gases and the like.

In alternative embodiments, carriers used to manufacture or formulate therapeutic combinations of drugs as provided herein, and drugs used to practice methods as provided herein, can be mixed as needed with disintegrants, diluents, granulating agents, lubricants, binders and the like using conventional techniques known in the art. The carriers can also be sterilized using methods that do not deleteriously react with the compounds, as is generally known in the art.

The invention also provides articles of manufacture and kits containing (comprising) therapeutic combinations of drugs as provided herein, and drugs used to practice methods as provided herein, including pharmaceutical compositions and formulations. By way of example only a kit or article of manufacture can include a container (such as a bottle) with a desired amount of a compound (or pharmaceutical composition of a compound) described herein. Such a kit or article of manufacture can further include instructions for using the compound (or pharmaceutical composition of a compound) described herein. The instructions can be attached to the container, or can be included in a package (such as a box or a plastic or foil bag) holding the container.

The therapeutic combinations of drugs as provided herein, and drugs used to practice methods as provided herein, can be delivered to the body or targeted to a specific tissue or organ (for example, a muscle or a brain) by any method or protocol, for example, including ex vivo “loading of cells” with therapeutic combinations of drugs as provided herein, and drugs used to practice methods as provided herein, where the “loaded cell” is the administered intramuscularly, or intrathecally, intracerebrally, or epidurally into the central nervous system (CNS), for example, as described in U.S. Pat. App. Pub. No. 20050048002.

In alternative embodiments therapeutic combinations of drugs as provided herein, and drugs used to practice methods as provided herein, are first lyophilized and then suspended in a hydrophobic medium, for example, comprising aliphatic, cyclic or aromatic molecules, for example, as described in U.S. Pat. App. Pub. No. 20080159984.

In alternative embodiments, therapeutic combinations of drugs as provided herein, and drugs used to practice methods as provided herein, comprise or are formulated as pharmaceutically acceptable salts. Pharmaceutically acceptable salts can include suitable acid addition or base salts thereof. In alternative embodiments, compounds can be formulated as described in Berge et al, J Pharm Sci, 66, 1-19 (1977).

In alternative embodiments, therapeutic combinations of drugs as provided herein, and drugs used to practice methods as provided herein, are formulated as salts that are formed, for example, with strong inorganic acids such as mineral acids, for example hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkane-carboxylic acids of 1 to 4 carbon atoms which are unsubstituted or substituted (for example, by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Compounds of the invention also encompass salts which are not pharmaceutically acceptable, for example, a salt may still be valuable as an intermediate in a synthetic or analytical process or protocol.

In alternative embodiments, therapeutic combinations of drugs as provided herein, and drugs used to practice methods as provided herein, comprise any acceptable salt for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2 -hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Pharmaceutical compositions as disclosed herein can be prepared in accordance with methods well known and routinely practiced in the art. See, for example, Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978.

In some embodiments, therapeutic combinations of drugs as provided herein, and drugs used to practice methods as provided herein, are provided in the form of pharmaceutically acceptable salts comprising an amine that is basic in nature and can react with an inorganic or organic acid to form a pharmaceutically acceptable acid addition salt; for example, such salts comprise inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids; or optionally such pharmaceutically acceptable salts comprise sulfate, pyrosulfate, bisulfate, sulfite, bisulfite, phosphate, mono-hydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, .beta.-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like salts.

In alternative embodiments, therapeutic combinations of drugs as provided herein, and drugs used to practice methods as provided herein, comprise compositions manufactured under “Good manufacturing practice” or GMP, or “current good manufacturing practices” (cGMP), conditions.

### Formulating Patient-Specific Everolimus Blood Concentration Estimates Utilizing a Bayesian-Informed Pharmacokinetic Model.

Alternative embodiments leverage a pharmacokinetic (PK) model to closely mirror PK profiles of individual patients in a community setting. The illustrative PK model generator delineated herein is capable of devising considerably precise pharmacokinetic models founded on a sample set of patients with diverse body weights, heights, renal and liver functions, ages, genders, pharmacogenetic variations, and concurrent medication regimens.

Various embodiments utilize patient samples derived from one or more patient data sets collected in a community setting, and optionally in medical office, hospital and specialized testing centers. The selected samples are collected from patients prescribed bMED™ everolimus and utilizing the bMED™ kit, inclusive of time-stamped individual dosage amounts, time-stamped blood sample collection apparatus, and sampling methodologies, and optionally, from patient samples collected from patients specifically selected to partake in the blood extraction process to aid in model creation and utilizing the bMED™ kit, inclusive of time-stamped individual dosage amounts, time-stamped blood sample collection apparatus, and sampling methodologies, and optionally, utilizing venous blood sampling.

In differing embodiments, patient samples encompass data for patients with assorted body weights, heights, renal and liver functions, ages, genders, pharmacogenetic variations, and concomitant medication regimens. In some cases, patient data can be divided into brackets based on body weight and renal function, allowing for the creation of individual models for each bracket. The data could be partitioned based on pharmacogenetic variations as an alternative or additional measure.

Certain embodiments entail the collection of blood samples post-everolimus administration from each patient after specified time periods. The collected samples may vary in terms of the time of collection and/or the total number of collected samples. For example, additional samples might be collected post the initial test dosage.

In certain embodiments, the model generator establishes a PK patient model through a Bayesian analysis, incorporating prior knowledge of blood or plasma everolimus concentrations over time in the sampled patients following the administration of a test dose or minimum therapeutic dose. In some cases, the model generator is designed to examine each patient's recorded dosing history in conjunction with pre-administration blood or plasma everolimus concentration levels, thereby eliminating the need for washout data to formulate the PK models. Conversely, in other embodiments, the model generator employs patient washout data alongside post administration blood or plasma everolimus concentration levels to establish one or more pharmacokinetic models.

The illustrative model generator can create one or more PK models using patient sample data. It may amalgamate individual patient samples into one or more population profiles, which then serve as the basis for the respective pharmacokinetic model. It may also segregate the patient samples based on different concomitant medications, weights, renal functions, ages, and/or pharmacogenetic variations into discrete sets. The model generator can then conduct covariate and statistical modeling on the grouped patient samples of each set to create a population pharmacokinetic model for that set.

In the provided exemplary embodiment, the covariate model applied by the model generator establishes relationships between pharmacokinetic parameters and patient characteristics. The model generator might use a statistical model to determine variance in pharmacokinetic parameters among the sampled patients in addition to residual variance due to biological variability between patients, measurement inaccuracies, and errors within the fit of the sampled data to the pharmacokinetic model.

In various embodiments, the illustrative model generator is designed to execute covariate and statistical modeling using the nonlinear mixed effects modeling software NONMEM version 7.3. The first-order conditional estimation method with interaction is implemented for all model runs. Run management is facilitated using Pirana version 2.9.2. Visual predictive checks and bootstrapping is performed using Perl-speaks-NONMEM version 3.7.6. Data manipulation and visualization is executed using the software R version 3.2.0 and RStudio version 0.99.442, with the lattice, xpose4, and ggplot2 packages used for the latter.

In alternative embodiments, a two-compartment model is employed for pharmacokinetic modeling. In the PK analysis, everolimus concentrations are modeled simultaneously. One, or two-compartment models are tested for both the parent and metabolite. The structural model is selected based on an evaluation of the objective function value, precision of parameter estimates, diagnostic plots, and model stability.

In various embodiments, after devising one or more pharmacokinetic models, the illustrative model generator delivers the pharmacokinetic model(s) to the PK server. The transmission can occur over a private or public network. Alternatively, the model generator may store the models in a database accessible by the PK server via one or more interfaces.

In several embodiments, the model generator refines the models for each patient using previously recorded treatment information such as weight, serum creatinine, and dosing level and interval. In this way, the PK server can create patient-specific models accounting for the individual pharmacokinetic variance. Once a PK profile for a patient is generated, it can be transmitted to the drug monitoring tool.

Alternative embodiments implement a drug monitoring tool comprising a data receiver and an interactive user interface, configured to display a time-varying everolimus level of the patient, founded on an administered drug dose and the patient's PK profile.

In alternative embodiments, bMED everolimus is available for oral administration in the following dosage forms and strengths and supplied on a blister card that time-stamps the use of the single dose tablet, capsule, or pre-filled syringe of everolimus solution or the blood collection device.

In alternative embodiments, everolimus is formulated as tablets, geltabs, pills or capsules or pre-filled syringe for administration provide 0.2 mg, 0.25 mg, 0.3 mg, 0.4 mg, 0.5 mg, 0.6 mg, 0.7 mg, 0.85 mg, 1.05 mg, 1.25 mg, 1.5 mg, 1.8 mg, 2.1 mg, 2.5 mg, 3.0 mg, 3.6 mg, 4.3 mg, 5.0 mg, 6.0 mg, 7 mg, 8.5 mg, 10 mg, 12 mg, 15 mg, 18 mg, 21 mg, 25 mg, 30 mg, 35 mg, or 40 mg of everolimus.

### Formulating Patient-Specific Everolimus Blood Concentration Estimates Utilizing a Bayesian-Model Informed Pharmacokinetic Model

Alternative embodiments leverage a pharmacokinetic (PK) model to closely mirror PK profiles of individual patients in a community setting. The illustrative PK model generator delineated herein is capable of devising considerably precise pharmacokinetic models founded on a sample set of patients with diverse body weights, heights, renal and liver functions, ages, genders, pharmacogenetic variations, and concurrent medication regimens.

Various embodiments utilize patient samples derived from one or more patient data sets collected in a community setting, and optionally in medical office, hospital and specialized testing centers. The selected samples are collected from patients prescribed bMED™ everolimus and utilizing the bMED™ kit, inclusive of time-stamped individual dosage amounts, time-stamped blood sample collection apparatus, and sampling methodologies, and optionally, from patient samples collected from patients specifically selected to partake in the blood extraction process to aid in model creation and utilizing the bMED™ kit, inclusive of time-stamped individual dosage amounts, time-stamped blood sample collection apparatus, and sampling methodologies, and optionally, utilizing venous blood sampling.

In differing embodiments, patient samples encompass data for patients with assorted body weights, heights, renal and liver functions, ages, genders, pharmacogenetic variations, and concomitant medication regimens. In some cases, patient data can be divided into brackets based on body weight and renal function, allowing for the creation of individual models for each bracket. The data could be partitioned based on pharmacogenetic variations as an alternative or additional measure.

Certain embodiments entail the collection of blood samples post-everolimus administration from each patient after specified time periods. The collected samples may vary in terms of the time of collection and/or the total number of collected samples. For example, additional samples might be collected post the initial test dosage.

In certain embodiments, the model generator establishes a PK patient model through a Bayesian analysis, incorporating prior knowledge of blood or plasma everolimus concentrations over time in the sampled patients following the administration of a test dose or minimum therapeutic dose. In some cases, the model generator is designed to examine each patient's recorded dosing history in conjunction with pre-administration blood or plasma everolimus concentration levels, thereby eliminating the need for washout data to formulate the PK models. Conversely, in other embodiments, the model generator employs patient washout data alongside post administration blood or plasma everolimus concentration levels to establish one or more pharmacokinetic models.

The illustrative model generator can create one or more PK models using patient sample data. It may amalgamate individual patient samples into one or more population profiles, which then serve as the basis for the respective pharmacokinetic model. It may also segregate the patient samples based on different concomitant medications, weights, renal functions, ages, and/or pharmacogenetic variations into discrete sets. The model generator can then conduct covariate and statistical modeling on the grouped patient samples of each set to create a population pharmacokinetic model for that set.

In the provided exemplary embodiment, the covariate model applied by the model generator establishes relationships between pharmacokinetic parameters and patient characteristics. The model generator might use a statistical model to determine variance in pharmacokinetic parameters among the sampled patients in addition to residual variance due to biological variability between patients, measurement inaccuracies, and errors within the fit of the sampled data to the pharmacokinetic model.

In various embodiments, the illustrative model generator is designed to execute covariate and statistical modeling using the nonlinear mixed effects modeling software NONMEM™ version 7.3™ (University of California, San Francisco (UCSF)). The first-order conditional estimation method with interaction is implemented for all model runs. Run management is facilitated using PIRANA™ version 2.9.2™ (Certara). Visual predictive checks and bootstrapping is performed using Perl-speaks-NONMEM version 3.7.6. Data manipulation and visualization is executed using the software R version 3.2.0 and RSTUDIO™ version 0.99.442™ (Posit), with the lattice, xpose4, and ggplot2 packages used for the latter.

In alternative embodiments, a two-compartment model is employed for pharmacokinetic modeling. In the PK analysis, everolimus concentrations are modeled simultaneously. One, or two-compartment models are tested for both the parent and metabolite. The structural model is selected based on an evaluation of the objective function value, precision of parameter estimates, diagnostic plots, and model stability.

In various embodiments, after devising one or more pharmacokinetic models, the illustrative model generator delivers the pharmacokinetic model(s) to the PK server. The transmission can occur over a private or public network. Alternatively, the model generator may store the models in a database accessible by the PK server via one or more interfaces.

In several embodiments, the model generator refines the models for each patient using previously recorded treatment information such as weight, serum creatinine, and dosing level and interval. In this way, the PK server can create patient-specific models accounting for the individual pharmacokinetic variance. Once a PK profile for a patient is generated, it can be transmitted to the drug monitoring tool.

Alternative embodiments implement a drug monitoring tool comprising a data receiver and an interactive user interface, configured to display a time-varying everolimus level of the patient, founded on an administered drug dose and the patient's PK profile.

### Derivatized and Deuterated Compounds

In alternative embodiments, therapeutic combinations of drugs as provided herein, and drugs used to practice methods as provided herein, are derivatized analogs, for example, metabolically blocked or otherwise altered derivatives, including deuterated, hydroxylated, fluorinated or methylated analogs or derivatives, or any combination thereof.

With regard to deuterated compounds as provided herein, or as used to practice methods as provided herein, it will be recognized that some variation of natural isotopic abundance occurs in a synthesized compound depending upon the origin of chemical materials used in the synthesis. Thus, a preparation of a compound will inherently contain small amounts of deuterated isotopologues. For compounds as provided herein, or as used to practice methods as provided herein, which include pharmaceutical preparations and formulations, when a particular position is designated as having deuterium (“-D”), it is understood that the abundance of deuterium at that position is greater than, or substantially greater than, the natural abundance of deuterium, which is 0.015%. For example, alternative embodiments of the invention comprise analogs of therapeutic combinations of drugs as provided herein, and drugs used to practice methods as provided herein, having greater than 0.02%, or greater than about 0.1% deuterium. In one embodiment, the deuterium substitution, or “enrichment”, occurs at a specific position or positions. In one embodiment, the deuterium enrichment is no less than about 1%, 10%, 20%, 50%, 70%, 80%, 90% or 95% or more or between about 1% and 100%.

In alternative embodiments, the deuterated (or otherwise substituted) compounds as provided herein, or as used to practice methods as provided herein, have a slower rate of metabolism, for example, slower rate of hydroxylation, than a corresponding protonated (non-deuterated, non-substituted) compound.

In alternative embodiments, compounds as provided herein, or as used to practice methods as provided herein are deuterated not to alter their properties (for example, not to alter their solubility, metabolism or pharmacological properties), but rather to distinguish an immediate release versus a delayed release component of a controlled release product, thereby enabling optimization of the immediate and delayed release component separately. Alternatively, drugs can be deuterated to detect and alert the caregiver or physician to any uncontrolled or alternative use of generic or off-label drugs that could undermine the integrity of the artificial intelligence (AI)-enabled dosing system.

### Stereoisomers

In alternative embodiments therapeutic combinations of drugs as provided herein, and drugs used to practice methods as provided herein, exist as (comprise) individual respective stereoisomers that are substantially free from another possible stereoisomer. In alternative embodiments, the term “substantially free of other stereoisomers” as used herein means less than about 15%, 20%, 25%, 30%, 35%, 40%, 50% or 55% of other stereoisomers, or less than about 10% of other stereoisomers, or less than about 5% of other stereoisomers, or less than about 2% of other stereoisomers, or less than about 1% or less of other stereoisomers, or less than “X” % of other stereoisomers (wherein X is a number between 0 and 100, inclusive) are present. Methods of obtaining or synthesizing an individual enantiomer for a given compound are known in the art and may be applied as practicable to final compounds or to starting material or intermediates.

### Methods of Administration

In alternative embodiments, therapeutic combinations of drugs as provided herein, and drugs used to practice methods as provided herein, are administered by any or a variety of means including orally, parenterally, by inhalation spray, nasally, topically, intrathecally, intrathecally, intracerebrally, epidurally, intracranially or rectally. Therapeutic combinations of drugs as provided herein, and drugs used to practice methods as provided herein, can be administered with pharmaceutically acceptable carriers, adjuvants and vehicles. In alternative embodiments, therapeutic combinations of drugs as provided herein, and drugs used to practice methods as provided herein, are administered by injection routes, including a variety of infusion techniques. Intraarterial, intrathecal, intracranial, epidural, intravenous and other injections can include administration through catheters or pumps, for example, an intrathecal pump, or an implantable medical device (which can be an intrathecal pump or catheter).

In alternative embodiments, therapeutic combinations of drugs as provided herein, and drugs used to practice methods as provided herein, are administered by any known method or route, including by intranasal, intramuscular, intravenous, topical or oral, or combinations thereof, routes.

One embodiment comprises a product of manufacture comprising a pharmaceutical composition or a formulation, a sachet or a blister package, a lidded blister or a blister card or packet, a clamshell, a tray or a shrink wrap, or a kit, comprising: therapeutic combinations of drugs, pharmaceutical compositions or preparations as provided herein for oral administration.

In alternative embodiments, although all ingredients can be in one blister package, a sachet, a lidded blister or a blister card or packet, a clamshell, a tray or a shrink wrap, or a kit, separate ingredients can be formulated for example, for topical application, for oral or for topical application. Each ingredient can be either separately packaged, or can be formulated as one unit dose, for example, as one tube (for example, with gel, lotion etc.), ampoule, sachet, blister packette and the like.

### Dosages

In alternative embodiments, therapeutic combinations of drugs as provided herein, and drugs used to practice methods as provided herein, are formulated and administered in a variety of different dosages and treatment regimens, depending on the disease or condition to be ameliorated, the condition of the individual to be treated, the goal of the treatment, and the like, as to be routinely determined by the clinician, see for example, the latest edition of Remington: The Science and Practice of Pharmacy, Mack Publishing Co., supra.

In alternative embodiments, an effective amount of a drug or compound as provided herein, or a composition used to practice the methods as provided herein, including a stereoisomer, salt, hydrate or solvate, is between about 0.1 mg and about 20.0 mg per kg of body weight of the individual or subject (for example, patient). In another variation, the effective amount is between about 0.1 mg and about 10.0 mg per kg of body weight of the individual or subject (for example, patient) or between about 0.1 mg and about 5.0 mg per kg of body weight of the patient. Alternately, the effective amount is between about 0.2 mg and about 2 mg per kg of body weight of the individual or subject (for example, patient).

In alternative embodiments, an effective amount of a therapeutic combination of drugs as provided herein, or a drug used to practice methods as provided herein (for example, as a powder or a solid dosage, such as a pill, tablet or lozenge) is between about 0.05 mg and about 20.0 mg per kg of body weight of said individual, subject or patient; or about 0.1 mg and about 10.0 mg per kg of body weight of said individual, subject or patient; or is between about 0.1 mg and about 2.0 mg per kg of body weight; or is about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, or about 1.0 mg, per kg of body weight; or an effective amount of a drug or compound as provided herein, or a composition used to practice the methods as provided herein, is about 0.1 mg, about 0.15 mg, about 0.2 mg, about 0.25 mg or about 0.3 mg per kg of body weight.

In alternative embodiment, an effective amount (for example, as a solid dosage, such as a pill, tablet or lozenge) of a drug or compound as provided herein, or a composition used to practice the methods as provided herein, is between about 0.25 mg and about 100 mg, between about 0.5 mg and about 200 mg, or between about 1 mg and about 400 mg, between 4 mg and 4 grams; or is a solid dosage form comprising between about is between about 0.25 mg and about 100 mg, between about 0.5 mg and about 200 mg, or between about 1 mg and about 250 mg; or the solid dosage form comprises between about 5 mg and about 150; or the solid dosage form (for example, as a pill, tablet or lozenge) comprises between about 1 mg and about 75; or the solid dosage form comprises about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, or about 75 mg or about 2 grams.

### Dosage Forms and Strengths

In alternative embodiments, bMED deflazacort is available for oral administration in the following dosage forms and strengths and supplied on a blister card that time-stamps the use of the single dose tablet or capsule, or the blood collection device.

In alternative embodiments, deflazacort is formulated as tablets, pills, geltabs or capsules for administration provide either 6 mg, 7 mg, 8 mg, 9 mg, 10 mg, 12 mg, 14 mg, 16 mg, 18 mg, 20 mg, 23 mg, 26 mg, 30 mg, 34 mg, 38 mg, 43 mg, 48 mg, 54 mg, 60 mg, 66 mg, 72 mg or 80 mg of deflazacort.

In alternative embodiments, bMED ataluren is available for oral administration in the following dosage forms and strengths and supplied on a blister card that timestamps the use of the single dose sachet or capsule, or the blood collection device.

In alternative embodiments, ataluren is formulated as a sachet or as pills, tablets, geltabs or capsules for administration provide of ataluren at for example 50 mg, 60 mg, 70 mg, 85 mg, 105 mg, 125 mg, 150 mg, 180 mg, 210 mg, 250 mg, 260 mg, 310 mg, 375 mg, 450 mg, 525 mg, 650 mg, 775 mg, 925 mg, 1000 mg, 1100 mg, 1350 mg, 1600 mg, 1900 mg or 2300 mg of ataluren granules for oral suspension.

In alternative embodiments, for treating TSC-associated partial onset seizures, including methods for determining drug dosages for treating TSC-associated partial onset seizures: doses can be individualized based on achieving a steady state trough blood drug concentration of everolimus and calculation of the everolimus dose using pharmacokinetic formula or the use of a Bayesian Model Informed pharmacokinetic model.

Treatment of children and adults ages two years of age and older with TSC-associated partial-onset seizers. To attain a blood trough target for everolimus of between about 5 to 15 ng/mL or 10 ng/mL:
Starting dose of 5 mg/m² orally once daily,
After 1 or 2 weeks adjust dose to a target of about 10 ng/mL trough blood concentration of everolimus,
Re-adjust dose every 1 or 2 weeks until obtain a target of about 10 ng/mL trough blood concentration for everolimus for two consecutive periods,
Re-adjust the dose monthly to maintain the target trough blood concentration for everolimus.

### Treatment of TSC-Associated Partial-Onset Seizures

In alternative embodiments, treatment of children and adults ages two years of age and older with TSC-associated partial-onset seizures: to attain a blood AUC₀₋₂₄ target for everolimus of between about 50 to 1,000 ng/mL*h or 500 ng/mL*h;
Starting dose for the of 5 mg/m² orally once daily.
After 1 or 2 weeks adjust dose to a target of about 500 ng/mL*h AUC₀₋₂₄ blood concentration of everolimus,
Re-adjust dose every 1 or 2 until obtain a target of about 500 ng/mL*h AUC₀₋₂₄ blood concentration for everolimus for two consecutive periods,
Readjust the dose monthly to maintain the target AUC₀₋₂₄ blood concentration for everolimus.
   Treatment of TSC-Associated Partial-Onset Seizures with Dose Titration Phase to a Tolerable Dose

In alternative embodiments, treatment of children and adults ages two years of age and older with TSC-associated partial-onset seizers: To titrate everolimus to a trough blood drug concentration target of 12 ng/mL or a tolerable trough concentration:
Starting dose of 5.0 mg/m² orally once daily,
After 1 or 2 weeks adjust the dose to a target of 5 ng/mL to 8 ng/trough blood concentration,
After 1 or 2 weeks adjust the dose to a target of about 8 ng/mL to 12 ng/mL trough blood concentration,
After 1 or 2 weeks adjust the dose to a target of about 10 ng/mL to 15 ng/mL trough blood concentration,
Re-adjust dose every 1 or 2 weeks until a target of about 12 ng/mL trough blood concentration for everolimus for two consecutive periods is obtained,
In the event of intolerable side effects, reduce dose to the tolerable trough blood concentration,
Re-adjust the dose monthly to maintain the target 12 ng/mL trough blood concentration or the tolerable trough blood concentration for everolimus.
   Treatment of TSC-Associated Partial-Onset Seizures with Selection of a Safe Starting Dose

In alternative embodiments, treatment of children and adults ages two years of age and older with TSC-associated partial-onset seizers. To select a safe starting dose of everolimus and titrate to a trough blood drug concentration target of 12 ng/mL or a tolerable trough concentration:
Administer a Test Dose of 5.0 mg/m², draw blood samples, and calculate the Test Dose steady-state,
Starting dose equal to the target of about 5 ng/mL to 7 ng/mL*Test Dose amount/Test Dose steady-state,
After 1 or 2 weeks adjust the dose to about 8 ng/mL to 12 ng/mL trough*average of the actual dose/actual blood drug concentration and test dose/Test Dose steady-state,
After 1 or 2 weeks adjust the dose to about 10 ng/mL to 15 ng/mL trough*moving average of the actual doses/actual blood drug concentrations and test dose/Test Dose steady-state,
Re-adjust dose every 1 or 2 weeks until a moving average blood drug concentration of about 12 ng/mL trough blood concentration for everolimus at least two consecutive periods is obtained,
In the event of intolerable side effects, reduce dose to the tolerable trough blood concentration,
Re-adjust the dose monthly or quarterly to maintain the moving average of blood drug concentration of about 12 ng/mL trough blood concentration for everolimus.
   Treatment of TSC-Associated Partial-Onset Seizures with Safe and Accelerated Titration to an Effective and Tolerable Blood Drug Concentration

In alternative embodiments, treatment of children and adults ages two years of age and older with TSC-associated partial-onset seizers. To select a safe starting dose of everolimus and titrate to a trough blood drug concentration target of 12 ng/mL or a tolerable trough concentration:
Administer a Test Dose of 5.0 mg/m², draw blood samples, and calculate Test Dose steady-state,
Starting dose equal to the target of 5 ng/mL to 7 ng/mL*Test Dose amount/Test Dose steady-state, and, optionally, a loading dose,
After 1 or 2 weeks adjust the dose to about 8 ng/mL to 12 ng/mL trough*average of the actual dose/actual blood drug concentration and test dose/Test Dose steady-state, and, optionally, a loading dose,
After 1 or 2 weeks adjust the dose to about 10 ng/mL to 15 ng/mL trough*moving average of the actual doses/actual blood drug concentrations and test dose/Test Dose steady-state, and optionally, a loading dose,
Re-adjust dose every 1 or 2 weeks to obtain a moving average blood drug concentration of about 12 ng/mL trough blood concentration for at least two consecutive periods,
In the event of intolerable side effects, reduce dose to the tolerable trough blood concenitration,
Re-adjust the dose monthly or quarterly to maintain the moving average of blood drug concentration of about 12 ng/mL trough blood concentration for everolimus.

### Dosage Forms and Strengths

In alternative embodiments, bMED everolimus is available for oral administration in the following dosage forms and strengths and supplied on a blister card that time-stamps the use of the single dose tablet, capsule, or pre-filled syringe of everolimus solution or the blood collection device.

In alternative embodiments, everolimus is formulated as tablets, pills, geltabs or capsules or pre-filled syringe for administration provide 0.5 mg, 0.6 mg, 0.7 mg, 0.85 mg, 1.05 mg, 1.25 mg, 1.5 mg, 1.8 mg, 2.1 mg, 2.5 mg, 3.0 mg, 3.6 mg, 4.3 mg, 5.0 mg, 6.0 mg, 7 mg, 8.5 mg, 10 mg, 12 mg, 15 mg, 18 mg, 21 mg, 25 mg, 30 mg, 35 mg or 40 mg of everolimus.

In alternative embodiments, methods for treating TSC-associated renal angiomyolipomas, including methods for determining drug dosages for treating TSC-associated renal angiomyolipomas: doses can individualized based on achieving a steady state trough blood drug concentration of everolimus and calculation of the everolimus dose using pharmacokinetic formula or the use of a Bayesian Model Informed pharmacokinetic model.

Treatment of Adults with renal angiomyolipoma and tuberous sclerosis complex (TSC), not requiring immediate surgery, to attain a trough blood drug concentration of target of about 8 ng/mL or a tolerable dose.
Starting dose of 10 mg orally once daily,
After 1 or 2 weeks adjust dose to a target of about 8 ng/mL trough blood concentration of everolimus,
Re-adjust dose every 1 or 2 weeks until obtain a target of about 8 ng/mL trough blood concentration 2 to 4 week moving average for everolimus for two consecutive periods,
Re-adjust the dose monthly to maintain the quarterly moving trough blood concentration of 8 ng/mL for everolimus.

In alternative embodiments, methods for treating hormone receptor-positive, HER2-negative breast cancer, including methods for determining drug dosages for treating hormone receptor-positive, HER2-negative breast cancer: doses can individualized based on achieving a steady state trough blood drug concentration of everolimus and calculation of the everolimus dose using pharmacokinetic formula or the use of a Bayesian Model Informed pharmacokinetic model.

Treatment of_Postmenopausal women with advanced hormone receptor-positive, HER2-negative breast cancer in combination with exemestane after failure of treatment with letrozole or anastrozole. To attain a blood trough target for everolimus of between about 12 to 18 ng/mL or 15 ng/mL or a tolerable trough blood drug concentration.
Administer a Test Dose of 5.0 mg/m², draw blood samples, and calculate Test Dose steady-state,
Starting dose equal to the target of 5 ng/mL*Test Dose amount/Test Dose steady-state, and, optionally, a loading dose,
After 2 or 3 weeks adjust the dose to about 8 ng/mL to 12 ng/mL trough*average of the actual dose/actual blood drug concentration and test dose/Test Dose steady-state, and, optionally, a loading dose,
After 2 or 3 weeks adjust the dose to about 12 ng/mL to 18 ng/mL trough*6 to 9 week moving average of the actual doses/actual blood drug concentrations and test dose/Test Dose steady-state, and optionally,
Re-adjust dose every 2 or 4 weeks to obtain a 8 to 12 week moving average blood drug concentration of about 15 ng/mL trough blood concentration for at least two consecutive periods,
In the event of intolerable side effects, reduce dose to the tolerable trough blood concentration,
Re-adjust the dose monthly or quarterly to maintain the quarterly moving average of trough blood drug concentration of about 15 ng/mL for everolimus.

In alternative embodiments, methods for treating Juvenile Polyposis, including methods for determining drug dosages for treating Juvenile Polyposis: doses can individualized based on achieving a steady state trough blood drug concentration of everolimus and calculation of the everolimus dose using pharmacokinetic formula or the use of a Bayesian model informed pharmacokinetic model.

Treatment of infant and children ages 6 months and older with Juvenile Polyposis. To attain a blood trough target for everolimus of between about 3 to 7 ng/mL or 5 ng/mL or a tolerable trough blood concentration:
Administer a Test Dose of 5.0 mg/m², draw blood samples, and calculate the Test Dose steady-state,
Starting BID dose equal to the target of about 3 ng/mL to 7 ng/mL*Test Dose amount/Test Dose steady-state,
After 1 or 2 weeks adjust the BID dose to about 5 ng/mL trough*average of the actual dose/actual blood drug concentration and test dose/Test Dose steady-state,
Re-adjust dose every 1 or 2 weeks until a moving average blood drug concentration of about 5 ng/mL trough blood concentration for everolimus at least two consecutive periods is obtained,
In the event of intolerable side effects, reduce dose to the tolerable trough blood concentration,
Re-adjust the dose monthly or quarterly to maintain the quarterly moving average of the trough blood drug concentration of about 5 ng/mL for everolimus.

In alternative embodiments, methods for treating Pulmonary Arterial Hypertension (PAH), including methods for determining drug dosages for treating Pulmonary Arterial Hypertension (PAH): doses can individualized based on achieving a steady state trough blood drug concentration of everolimus and calculation of the everolimus dose using pharmacokinetic formula or the use of a Bayesian Model Informed pharmacokinetic model.

Treatment of adults with Pulmonary Arterial Hypertension. To attain a blood trough target for everolimus of between about 5 to 8 ng/mL or 6 ng/mL or a tolerable trough blood drug concentration:
Starting dose of 2.5 mg/m² orally twice daily (BID),
After 1 or 2 weeks adjust BID dose to a target of about 6 ng/mL trough blood concentration of everolimus,
Re-adjust BID dose every 1 or 2 weeks until obtain a target of about 6 ng/mL trough blood concentration for everolimus for two consecutive periods,
In the event of intolerable side effects, reduce dose to the tolerable trough blood concentration,
Re-adjust the dose monthly or quarterly to maintain the moving average of blood drug concentration of about 6 ng/mL trough blood concentration for everolimus.

### Packaging and Drug Delivery Systems

In alternative embodiments, provided are therapeutic combinations, preparations, formulations and/or kits, comprising combinations of ingredients, as described herein. In one aspect, each member of the combination of ingredients is manufactured in a separate package, kit or container; or, all or a subset of the combinations of ingredients are manufactured in a separate package or container. In alternative aspects, the package, kit or container comprises sachet, a blister package, a clamshell, a tray, a shrink wrap and the like.

In one aspect, the sachet, package, kit or container comprises a “blister package” (also called a blister pack, or bubble pack). In alternative embodiments, provided are therapeutic combinations, preparations, formulations and/or kits manufactured as “blister packages” or as a plurality of packettes, including as sachets, lidded blister packages, lidded blister or blister card or packets or packettes, or a shrink wrap.

In one aspect, the blister package is made up of two separate elements: a transparent or occlusive plastic cavity shaped to the product and its blister foil backing. These two elements are then sealed together into a blister strip of one or more blister with each blister an environmentally (for example moisture, pathogen, light) protected unit dose. One or more blister strips can be further joined with board material which allows the product to be package, handled, hung, displayed or shipped without damaging the blister seal and provided child resistant features. Exemplary types of “blister packages” include: Face seal blister packages, gang run blister packages, mock blister packages, interactive blister packages, slide blister packages.

Blister packs, sachets, clamshells or trays are forms of packaging used for goods; thus, provided are blister packs, clamshells or trays comprising a composition (for example, a (the multi-ingredient combination of drugs as provided herein) combination of active ingredients) as provided herein. Blister packs, sachets, clamshells or trays can be designed to be non-reclosable, so consumers can tell if a package has already opened. They are used to package for sale goods where product tampering is a consideration, such as the pharmaceuticals as provided herein. In one aspect, a blister pack as provided herein comprises a molded PVC base, with raised areas (the “blisters”) to contain the tablets, pills, etc. comprising the combinations as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In alternative embodiments, laminated aluminum foil blister packs are used, for example, for the preparation of drugs designed to dissolve immediately in the mouth of a patient. This exemplary process comprises having the drug combinations, therapeutic combinations and pharmaceutical dosage forms as provided herein prepared as an aqueous solution(s) which are dispensed (for example, by measured dose) into an aluminum (for example, alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system gives tablets an identification mark by embossing a design into the alufoil pocket that is taken up by the tablets when they change from aqueous to solid state. In one aspect, individual ‘push-through’ blister packs/packettes are used, for example, using hard temper aluminum (for example, alufoil) lidding material. In one aspect, hermetically-sealed high barrier aluminum (for example, alufoil) laminates are used. In one aspect, any products of manufacture as provided herein, including kits or blister packs, use foil laminations and strip packs, stick packs, sachets and pouches, peelable and non-peelable laminations combining foil, paper, and film for high barrier packaging.

In alternative embodiments, any products of manufacture as provided herein, including kits sachets, or blister packs, include memory aids to help remind patients when and how to take the drug. This safeguards the drug's efficacy by protecting each pill until it's taken; gives the product or kit portability, makes it easy to take a dose anytime or anywhere.

In alternative embodiments, drug combinations, therapeutic combinations, pharmaceutical dosage forms, drug delivery devices and products of manufacture as provided herein, use child resistant and elderly friendly packaging, for example, packaging compliant to U.S. Government child resistant packaging regulation that requires minimal finger and grip strength. For example, in alternative embodiments foil-only containment of pills is used.

In alternative embodiments, drug combinations, therapeutic combinations, pharmaceutical dosage forms, drug delivery devices and products of manufacture as provided herein, are manufactured or packaged in or as sachets, tablets, capsules, pills or equivalents on a blister card or equivalent to track usage. By tracking usage, the package (for example, sachet or blister card) monitor can remind the patient and/or the primary care-giver to take medication (or that medication has been taken) at the correct time, for example, in AM and/or PM; and can facilitate discussion with health care professionals to identify and overcome barriers to adherence.

In alternative embodiments, patient usage is monitored by use of customized blister cards or equivalents using an Electronic Compliance Monitor (ECM) system (Intelligent Devices SEZC Inc. (IDI), Grand Cayman, Cayman Islands), or equivalents. For example, in alternative embodiments, the blister cards or equivalents comprise an electronic component that detects, records, safeguards and/or transmits medication removal from the blister cards or equivalents. For example, a sensor detects medication removal from the blister cards or equivalents, and this information can be transferred to a remote location for review by for example, the drug provider and/or the primary care institution or individuals. The data transfer can be by hard contact downloading of data to a transmitting and/or storage device, and can be scanned and data downloaded remotely using a radio-frequency identification (RFID) chip, tag or device or equivalent, which can be operatively connected to a computer and/or a mobile phone or other device. Radio-frequency identification uses electromagnetic fields to automatically identify and track tags attached to objects, where the tags contain electronically stored information, which in this embodiment is transmitting whether and/or when medication is removed from each compartment of the blister cards or equivalents, or by Near-Field Communication (NFC) to a NFC-enabled mobile device or mobile phone. The NFC is a set of communication protocols that enable two electronic devices, one of which is usually a portable device such as a smartphone, to establish communication by bringing them within 4 cm (1.6 in) of each other.

In alternative embodiments, multi-drug delivery systems as used in methods as provided herein can comprise use of a box to house or enclose drug delivery devices or packages, sachets, blister packages, clamshells or trays, as provided herein, where in this exemplary delivery system a week of pharmaceutical dosage form (for example, one, two or three or more tablets, pills, capsules, geltabs or equivalents) are stored on four rows, two rows for administration (for opening and self-administering by user, for example, patient) are for morning or breakfast, or AM administration, and two rows are for evening, dinnertime or PM administration; morning or breakfast, or AM administration rows are clearly separated from the evening, dinnertime or PM administration rows, and each day, and the spare dose, are arranged in column form. In alternative embodiments the blister packages, clamshells or trays are physical linked to a storage box, wherein the blister packages, clamshells or trays slide into and out of the storage box, and in alternative embodiments if needed the PM set of rows can be folded over the AM set of rows for reinsertion of the blister packages, clamshells or trays into the storage box. In alternative embodiments, the storage box comprises sensors to detect medication removal from each of the compartments (for example, which compartment is opened and when), and this information can be transferred to a remote location, for example, by Near-Field Communication (NFC) to a NFC-enabled mobile device or mobile phone, for review by for example, the drug provider and/or the primary care institution or individuals.

In any embodiment of methods as provided herein, the blood or plasma concentration for the drug, or therapeutic combination, pharmaceutical dosage form is: (a) the trough level or trough concentration (C_{trough}), or the lowest concentration reached by the drug, or therapeutic combination or pharmaceutical dosage form before a second or next dose is administered, or (b) determined from blood samples taken between about 0.25 hours to 48 hours or 0.5 hours to 24 hours, or 4 to 12 hours, or 1, 2, 3, 4, 5, 6, 7, 8, 9, or 10 or more hours, after the last dose or administration of the drug, or therapeutic combination, pharmaceutical dosage form.

In alternative embodiments, provided are kits comprising:
everolimus or sirolimus (also known as rapamycin) packaged as or in individual dosed units such as capsules, sachets, pills, geltabs, tablets or film products,
and the individual dosed units (or capsules, pills, geltabs, tablets or film products) are contained in a smart blister card; and
and optionally the kits comprise a dried blood spot device, a volumetric dried blood spot, volumetric absorptive microsampling (VAMS) or the at-home high-volume liquid blood collection device, and optionally the blood collection device comprises a hand-held device comprising a plurality of microneedles or volumetric needles, wherein the plurality of microneedles or volumetric needle are capable of capturing a predefined volummetric blood sample, for example, a blood sample of between about 5 μl and 500 μl, or between about 10 μl and 100 μl, or about 5 μl, 10 μl, 15 μl, 20 μl, 25 μl, 30 μl or 35 μl, and optionally the volumetric dried blood spot, volumetric absorptive microsampling (VAMS) or the at-home high-volume liquid blood collection device comprises a MITRA™ device (by NEOTERYX™), or a bladeless microneedle array as in a TAPMICRO™ or HALO™ device (yourbio, Medford, MA),
and optionally the dried blood spot device is contained or fabricated in a smart blister card,
and the everolimus or sirolimus are dosaged for the treatment of breast cancer, a neuroendocrine tumor (NET), renal cell carcinoma (RCC), tuberous sclerosis complex (TSC)-associated renal angiomyolipoma, TSC-Associated subependymal giant cell astrocytoma (SEGA), TSC-associated partial-onset seizures, prophylaxis of organ rejection in renal and liver transplants; lymphangioleiomyomatosis (LAM), Leigh syndrome, pulmonary arterial hypertension (PAH), familial adenomatous polyposis (FAP), juvenile polyposis syndrome (JPS),
and optionally the everolimus is dosaged to attain a predicted blood C₂₄ₕ target for everolimus of between about 1 to 20 ng/mL, between about 5 to 15 ng/ml, between about 10 to 15 ng/mL or 12 ng/mL and to obtain a predicted blood or plasma AUC target precent coefficient of variation between about 5% to 35%, between about 5% to 25, between about 5% to 15% or between about 5% to 10%,
and optionally the sirolimus (also known as rapamycin) is dosaged to attain a predicted blood C24h target for sirolimus of between about 1 to 20 ng/mL, between about 5 to 15 ng/ml, between about 10 to 15 ng/mL or 12 ng/mL and to obtain a predicted blood or plasma AUC target precent coefficient of variation between about 5% to 35%, between about 5% to 25, between about 5% to 15%, or between about 5% to 10%.

In alternative embodiments, provided are kits comprising:
ataluren and deflazacor packaged as or in individual unit dosages such as capsules, sachets, pills, geltabs, tablets or film products, or as a liquid formulation,
and optionally the kits comprise a dried blood spot device, a volumetric dried blood spot, volumetric absorptive microsampling (VAMS) or the at-home high-volume liquid blood collection device,
and optionally the blood collection device comprises a hand-held device comprising a plurality of microneedles or volumetric needles, wherein the plurality of microneedles or volumetric needle are capable of capturing a predefined volummetric blood sample, for example, a blood sample of between about 5 μl and 500 μl, or between about 10 μl and 100 μl, or about 5 μl, 10 μl, 15 μl, 20 μl, 25 μl, 30 μl or 35 μl, and optionally the volumetric dried blood spot, volumetric absorptive microsampling (VAMS) or the at-home high-volume liquid blood collection device comprises a MITRA™ device (by NEOTERYX™), or a bladeless microneedle array as in a TAPMICRO™ or HALO™ device (yourbio, Medford, MA),
and the individual dosed units (or capsules, pills, geltabs, tablets or film products, or liquid formulation) are contained in a smart blister card, sachet or packette, and optionally both the ataluren and deflazacort unit dose are packaged or contained in one or a single container or receptacle; and
a dried blood spot device, and optionally the dried blood spot device is contained or fabricated in a smart blister card,
and the ataluren is dosage for the treatment of Duchenne Muscular Dystrophy,
and optionally the ataluren is dosaged to attain a predicted blood or plasma C6h target or a trough blood or plasma concentration target prior to the evening dose for ataluren of between about 1 to 20 μg/mL, between about 2 to 15 μg/mL, between about 5 to 10 μg/mL or 7 μg/mL, and to obtain a predicted blood or plasma AUC target precent coefficient of variation between about 5% to 35%, between about 5% to 25, between about 5% to 15%, or between about 5% to 10%,
and optionally the deflazacort (or EMFLAZA™, CALCORT) is to attain a blood or plasma AUC0-4 h target or AUC0-8 h target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or 250 ng/mL*h8; deflazacort is to attain a blood or plasma AUC0-infinity target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or 250 ng/mL*h and to obtain a predicted blood or plasma AUC target precent coefficient of variation between about 5% to 35%, between about 5% to 25, between about 5% to 15% or between about 5% to 10%.

### Treating Proliferating Infantile Hemangioma

In alternative embodiments, doses are individualized based on trough blood or plasma propranolol concentration and use of a Bayesian Model Informed pharmacokinetic model.

In alternative embodiments, treatment of Proliferating Infantile Hemangioma (IH), or PIH comprises:
Initiate treatment at ages 5 weeks to 5 months.
Starting dose is 0.15 mL/kg propranolol hydrochloride oral solution (0.6 mg propranolol/kg) twice daily.
After 3 days adjust dose twice daily to a target of between about 20 ng/mL to 30 ng/mL C₉ₕ or trough blood or plasma propranolol concentration after the AM dose or trough blood or plasma prior to the PM dose;
After 1 week increase dose twice daily to a target of 40 ng/mL to 50 ml C₉ₕ or trough blood or plasma propranolol concentration;
After 2 weeks increase dose twice daily to a target of 60 ng/mL to 80 ng/mLC₉ₕ or trough blood or plasma propranolol concentration;
Readjust dose monthly to a target of 60 ng/mL to 80 ng/mL C₉ₕ or trough blood or plasma propranolol concentration;
Monitor heart rate and blood pressure for about 2 hours after the first dose or increasing dose.
   In alternative embodiments, maintenance treatment of Infantile Hemangioma (IH) comprises:
Titrate dose to attain a C₉ₕ or trough blood or plasma propranolol concentration of between about 20 ng/mL to 30 ng/mL;
Readjust dose monthly to a target of 20 ng/mL to 30 ng/mL C₉ₕ or trough blood or plasma propranolol concentration.

Consider tapering bMED propranolol after 6 months of maintenance treatment.
Taper bMED propranolol one C₉ₕ or trough blood or plasma propranolol concentration every 2 weeks (to between about 15 ng/mL to 20 ng/mL to between about 10 ng/mL to 15 ng/mL to between about 5 to 10 ng/mL);
If infantile hemangioma (IH) signs (for example, angiopoietin 2, tumor growth) recur during or after the taper of bMED propranolol, consider retreatment.

In alternative embodiments, evening dose administration is withheld during fever or infection and resumed upon resolution of the fever or infection.

### Dose and Blood Sample Collection Timing for Trough Blood Concentration Determination

In alternative embodiments, calculation of trough blood or plasma drug concentration is dependent on the amount and timing of prior drug administration, the timing of acute illness, the timing of blood sample collection, the blood collection device, the laboratory assay method and an individualized and adaptive pharmacokinetic model of drug clearance.

In alternative embodiments, drug administration occurs immediately after pre-filled syringe is removed from the syringe blister card (that has a seven day supply of pre-filled syringes).

In alternative embodiments, the blood sample collection should occur prior to drug administration of the last syringe on the blister card.

In alternative embodiments, the blood sample should occur immediately after the blood collection device is removed from the blood collection blister card.

In alternative embodiments, the blood sample can be placed in the blood sample pouch.

In alternative embodiments, the pouch, the empty drug blister card and the empty device card can be placed in shipping box and sent to the designated assay laboratory for the determination of blood sirolimus concentrations.

### Calculation of Blood or Plasma Propranolol Calculation Using Bayesian Model Informed Pharmacokinetic Model

In alternative embodiments, a pharmacokinetic (PK) model is used to approximate pharmacokinetic (PK) profiles of patients. For instance, current methods to determine a patient-specific pharmacokinetic profile for infantile hemangioma include performing multiple blood tests. After (test dose, minimum therapeutic dose) of propranolol is administered, five or more blood draws are performed over 9-hour post-administration period. As can be appreciated, such a procedure is especially taxing on a patient, healthcare provider, and lab because of the numerous separate blood draws.

In alternative embodiments, the exemplary model generator as provided herein is configured to generate relatively accurate pharmacokinetic models based upon a sample of patients with varying body weights, postnatal age, postmenstrual age, gestational age, height, renal function, liver function and genders. These models are then used to determine or approximate a pharmacokinetic profile of a patient without having to subject a patient to all of the blood draws and subsequent analysis.

In alternative embodiments, PK models are determined using patient samples selected from one or more sets of patient data. The patient samples may be, for example, selected among patients who have already been subscribed a therapeutic dosing regimen using the above-described blood draw procedure. The patient samples may also include patients specifically selected to go through the blood draw procedure for the purpose of creating the models. The patient samples may include patients from one hospital or medical system and/or patients associated from multiple hospitals, medical systems, geographic regions, etc.

In alternative embodiments, patient samples include data for patients of varying postnatal ages, body weights (or body mass index (“BMI”), medical conditions, clinical laboratory data, and genders. In alternative embodiments, patients' ages vary between postnatal age of 53 to 150 days. In some embodiments, the data for the patients may be separated into age brackets such that a separate model is generated for each bracket. The patient data may additionally or alternatively be partitioned based on weight and/or genders.

In alternative embodiments, post-drug administration (for example, post-propranolol administration) blood samples are collected from each patient after certain durations of time. In alternative embodiments, the blood samples are collected at different times and/or the number of blood samples collected are fewer or greater. For instance, fewer blood samples may be collected from newborns.

In alternative embodiments, the exemplary model generator creates a PK patient model by performing a Bayesian analysis that uses previous knowledge of blood or plasma propranolol concentration in the sampled patients over time after administration of the (test dose, minimum therapeutic dose). In some instances, he model generator is configured to analyze each patient's sampled dosing history in conjunction with pre-administration blood or plasma propranolol concentration levels, so that washout data is not needed to construct the PK models. In other embodiments, the model generator uses patient washout data in conjunction with the post administration blood or plasma propranolol concentration levels to create one or more pharmacokinetic models. Patient washout data corresponds to a baseline where the patient does not include propranolol in their system.

In alternative embodiments, the exemplary model generator creates the one or more PK models using, for example, the patient sample data. The model generator may combine the individual patient samples into one or more population profiles (for example, age sets, weight sets, sex, etc.), which is then used as a basis for the respective pharmacokinetic model. In alternative embodiments, the model generator groups the patient samples for different ages, weights, and/or sex into different sets. In alternative embodiments, the model generator then performs covariate and statistical modeling on the grouped patient samples of each set to create a population pharmacokinetic model for that set, for example, as described by Takechi et al. (2018) “Population Pharmacokinetics and Pharmacodynamics of Oral Propranolol in Pediatric Patients With Infantile Hemangioma”, Clinical Pharmacology, vol 58(10), pg 1361-1370, the entirety of which is incorporated herein by reference. In alternative embodiments the model generator uses a model and sampled data from other Bayesian analysis techniques (for example, a naïve Bayes classifier).

In alternative embodiments, the covariate model used by the model generator determines relationships between pharmacokinetic parameters (for example, how quickly propranolol is metabolized, etc.) and patient characteristics (for example, postnatal age, body weight, clinical laboratory data, gender, etc.). In alternative embodiments, the model generator uses a statistical model to determine variance in pharmacokinetic parameters among the sampled patients in addition to residual variance as a result of biological variability between patients, measurement errors, and errors within the fit of the sampled data to the pharmacokinetic model.

In alternative embodiments, the exemplary model generator is configured to perform the covariate and statistical modeling using the nonparametric bootstrap approach with Perl-speaks-NONMEM (PsN) version 4.4.8™ (see for example, Lindbom et al (2004) Computer Methods Programs in Biomed., vol. 75(2), pg 85-94).

The ability of the final population pharmacokinetic model to describe observed concentration data was evaluated by visual predictive check. A total of 1000 simulations were performed with the final population pharmacokinetic model, and the 90% prediction interval (PI) was calculated and used to overlay the observed data for visual predictive check.

### Clinical Trial Simulations and Modeling

Clinical trial simulations and modeling methodologies were utilized to design innovative titration and maintenance dosing algorithms for Everolimus, a treatment for TSC-associated epilepsy in both adult and pediatric populations. The algorithms were designed to optimize drug blood concentrations, ensuring efficacy and safety. These simulations were also used to compare the novel dosing algorithms to existing protocols.

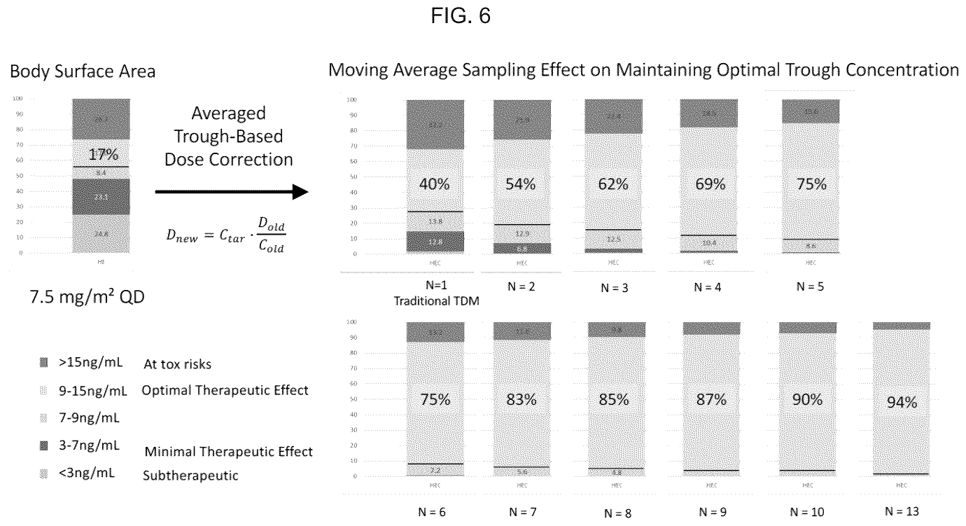

Initially, a population pharmacokinetic (PK) model, termed “TSC-MODEL”, was devised based on a previously validated and published PK model (the “COMBES MODEL”, see Combes et al (2020) Pharmacometrics & Systems Pharmacology, Vol 9(4), pages 230-237), established using EXIST-3 (a Three-arm, Randomized, Double-blind, Placebo-controlled Study of the Efficacy and Safety of Two Trough-ranges of Everolimus as Adjunctive Therapy in Patients With Tuberous Sclerosis Complex (TSC) Who Have Refractory Partial-onset Seizures (Phase III)-coordination, Novartis Pharma) clinical study data. The model parameters are depicted in FIG. 6. The COMBES MODEL illustrated a significant relationship between blood drug concentration exposure and seizure frequency with optimal response at a trough blood drug concentration at 9 ng/mL to 15 ng/mL. It also demonstrated considerable inter-patient and intra-patient variability in blood drug concentration.

Next, the TSC MODEL and COMBES MODEL were compared for validation purposes. The comparison confirmed model equivalence, with population demographics also aligning closely.

Following model validation, a simulation of 10,000 patients was conducted using the TSC MODEL to generate a distribution of blood drug concentrations. The model results were consistent with the COMBES model results, further confirming model robustness.

Subsequently, the TSC MODEL was evaluated against a validated PK model for renal transplant disease states treated with Everolimus (“ZWART MODEL”, see Zwart et al, Clin Pharmacokinet (2021) February; vol 60(2):191-203). Comparison of simulated patient groups, using both models, yielded approximately equivalent blood drug concentration distributions, demonstrating the versatility of the TSC MODEL across TSC-related diseases.

In the next phase, the validated TSC MODEL was employed to simulate 10,000 patients receiving Everolimus based on different dosing algorithms: fixed dosing, body surface area-based dosing, traditional therapeutic drug monitoring, and monitoring using moving averages of various blood drug concentration measurements.

The results, as depicted in EXHIBIT 2 demonstrated the effectiveness of the moving average algorithm in maintaining optimal blood drug concentrations with significantly reduced risk of subtherapeutic or potentially toxic doses, compared to label and published recommendations.

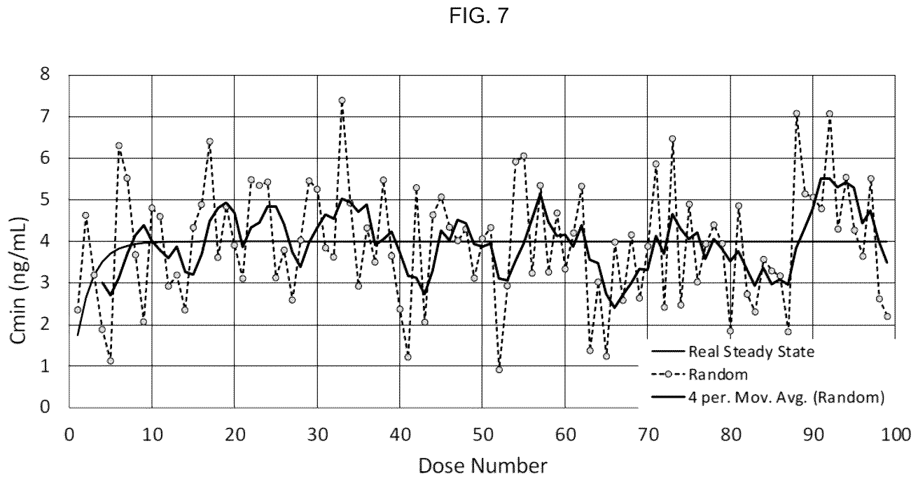

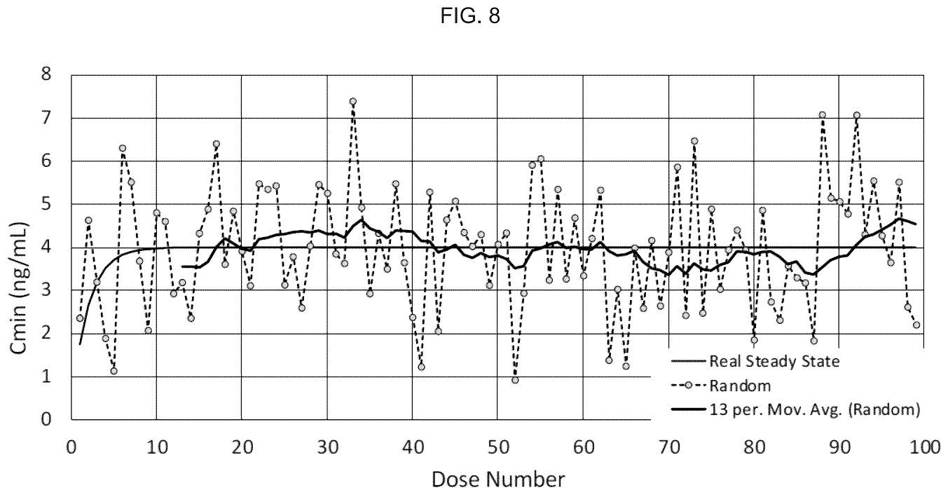

FIG. 7 and FIG. 8 further detail the time course of an exemplary patient's measurements, illustrating how the 4-period and 13-period moving averages dampen random fluctuation of single measurement blood-drug concentration, facilitating accurate dose adjustment and minimizing unnecessary dosage alterations.

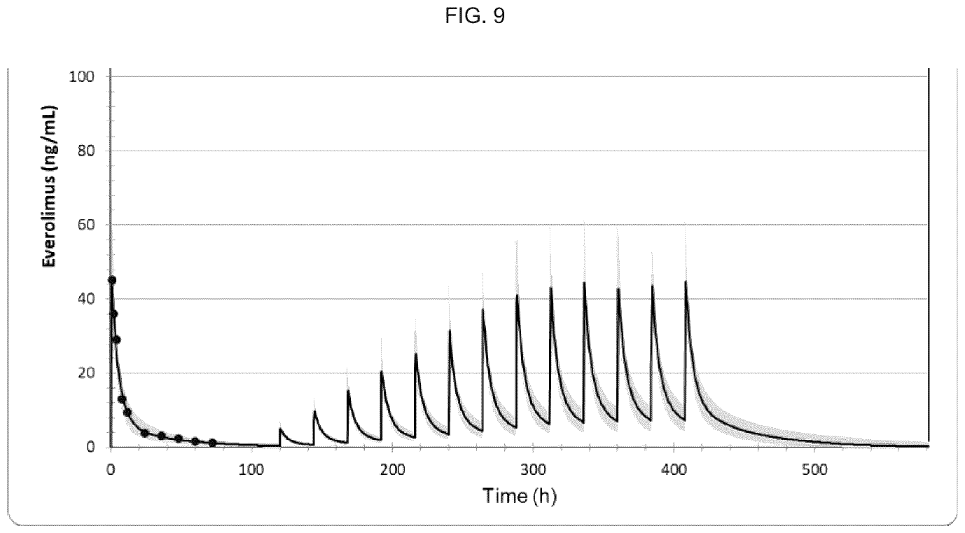

FIG. 9 depicts the 95% Confidence Interval of Predicted Blood Drug Concentration Using Test Dose Method. The Simulation of 10,000 Samples shows the test dose method enables intermediate metabolizing patient to reach the target trough blood concertation within 10 days.

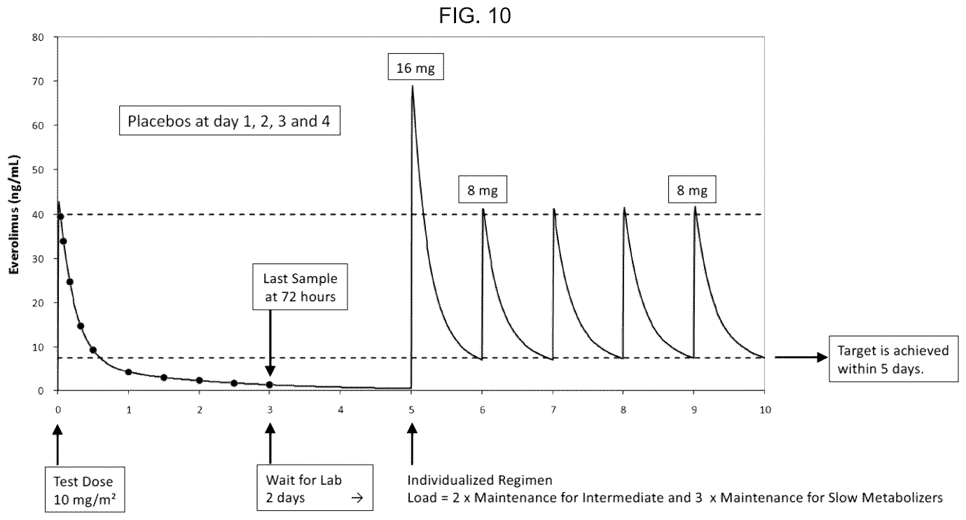

FIG. 10 depicts the Use of The Test Dose Method Combined with a Loading Dose enabling intermediate and slow metabolizing patients to reach the target trough blood concentration of 5 ng/mL after first day of receiving the maintenance plus loading dose.

In alternative embodiments, a one-compartment model is used for pharmacokinetic modeling. The first-order absorption rate constant (kₐ) is fixed at the value calculated according to the equation tₘₐₓ=Ln(kₐ/kₑₗ)/(kₐ−kₑₗ), using previously reported pharmacokinetic parameters because of a lack of sampling points. Interindividual variability on each parameter assumed a log-normal distribution and was estimated with an exponential error model. An additive error model, a proportional error model, and a combination of additive and proportional error models were investigated to describe the residual variability. A basic model was selected using the Akaike information criterion. Body weight, postnatal age, postmenstrual age, height, serum creatinine, aspartate aminotransferase, alanine aminotransferase, and sex were examined as candidates for pharmacokinetic covariates. As a structural covariate, a model using fixed theoretical exponents (0.75 for CL/F and 1 for apparent central volume of distribution [V/F]) with body weight was included in the basic model to account for the dose calculation (in mg/kg) and growth and development of the infants. The selection of covariates to be included in the model was conducted using stepwise forward selection (P<0.05), followed by backward elimination (P<0.01), based on changes in objective function value (OFV). Continuous covariates were modeled with a power function centered on a median value. A categorical covariate was incorporated as a discrete indicator variable. The adequacy of the constructed population pharmacokinetic model was assessed using goodness-of-fit plots. Goodness-of-fit was investigated using plots of observations versus population prediction (PRED) and individual prediction (IPRED), conditional weighted residuals versus time after the first administration and time after the last administration, conditional weighted residuals versus PRED and absolute individual weighted residuals versus IPRED. Shrinkage in random effects was computed to guide the appropriateness of using ETA (I, empirical Bayes estimate of the interindividual random effect) and IPRED values in the goodness-of-fit assessment.

In alternative embodiments, responsive to creating one or more pharmacokinetic models, the exemplary model generator provides the pharmacokinetic model(s) to the PK server. The transmission may be over a private network, such as a local area network, or over a public network, such as an Internet. The model generator may also store the models to the database, which is also accessible by the PK server via one or more interfaces. In other instances, the model generator may be integrated with the PK server.

In alternative embodiments, the exemplary model generator refines the models for each patient. For instance, the PK server may receive patient specific information including, weight, age, gender, and dosing level for previous treatments. The model generator uses the previous treatment information (for example, dosing amounts, intervals, etc.) to refine or adjust the model such that dosing recommendations and a pharmacokinetic profile are more aligned to the specific patient but still account for potential patient variance. The model generator transmits the patient-specific model to the PK server.

In alternative embodiments, the PK server is configured to create patient-specific models using the pharmacokinetic model provided by the model generator to account for the patient-specific pharmacokinetic variance. In this manner, one or more base models are refined or adjusted by the PK server responsive to receiving previous treatment information for a specific patient. The PK server may be configured to store the patient-specific model to the database for subsequent uses by the same healthcare provider or other healthcare providers.

In alternative embodiments, once a PK profile for a patient is generated, the PK server is configured to transmit the PK profile to the drug monitoring tool. In some embodiments, the PK server can encrypt the data file prior to transmission. The encryption can be specific to a particular patient such that the drug monitoring tool can only open and process a received PK profile if the tool has a patient specific authentication key.

In alternative embodiments, methods as provided herein use a drug monitoring tool as described by U.S. Pat. No. 10,896,749, which comprises a data receiver and an interactive user interface, where the data receiver is configured to receive a pharmacokinetic (PK) profile of a patient, and an interactive user interface is configured to display a time-varying therapeutic drug or plasma protein level of the patient. The time-varying therapeutic drug or plasma protein level is based on an administered dose of a drug, and the PK profile of the patient.

In alternative embodiments, the drug monitoring tool used in methods as provided herein comprises: a data receiver configured to receive, from a secured server, a patient pharmacokinetic (PK) profile of a patient, wherein the secured server comprises: (1) a Bayesian model of pharmacokinetic (PK) profiles of sampled patients, the Bayesian model including (i) a blood or plasma drug clearance and (ii) a volume of distribution relationship for a plasma protein or drug based upon at least one of patient age or body weight, and (2) a PK server configured to determine the patient PK profile based upon the Bayesian model, and at least one of a bodyweight, height, and an age of the patient; and an interactive user interface configured to: display to the patient a graphical representation predictive of a time-varying drug level in the patient, the time-varying drug level determined by the drug monitoring tool based on the timing and amount of an administered dose of the drug to the patient and the patient PK profile.

### Dosage Forms and Strengths

In alternative embodiments, a drug, drug combination or drug formulation as provided herein, for example, comprising bMED Propranolol, is available for oral administration in the following dosage forms and strengths and supplied on a blister card that time-stamps the use of the single dose pre-filled syringe or capsule, or the blood collection device.

In alternative embodiments, a drug, drug combination or drug formulation as provided herein, for example, comprising bMED Propranolol, are formulated as or in granules, for example, as granules for oral suspension. Granules for oral suspension in single dose pre-filled 5 mL syringes, when reconstituted, provide either 0.05 mg/mL, 0.075 mg/mL, 0.1 mg/5 mL, 0.15 mg/5 mL, 0.2 mg/5 mL, 0.25 mg/5 mL, 0.3 mg/5 mL, 0.4 mg/5 mL, 0.5 mg/5 mL, 0.6 mg/5 mL, 0.8 mg/5 mL, 1.0 mg/5 mL, 1.25 mg/5 mL, 1.5 mg/5 mL, 2 mg/5 mL, 2.5 mg/5 mL, 3 mg/5 mL, 3.5 mg/5 mL, 4 mg/5 mL, 4.5 mg/5 mL, 5 mg/5 mL, 5.5 mg/5 mL, 6 mg/5 mL, 6.5 mg/5 mL, 7 mg/5 mL, 7.5 mg/5 mL, 8 mg/5 mL, 8.5 mg/5 mL, 9 mg/5 mL, 9.5 mg/5 mL, 10 mg/5 mL, 11 mg/5 mL, 12 mg/5 mL, 13 mg/5 mL, 14 mg/5 mL, 15 mg/5 mL, 16 mg/5 mL, 17 mg/5 mL, 18 mg/5 mL, 20 mg/5 mL, 22 mg/5 mL, 24 mg/5 mL, 26 mg/5 mL, 28 mg/5 mL or 30 mg/5 mL or 2.0 mg/mL of propranolol. The granules have an off white to cream color and are strawberry flavored.

For P2 study consider use of oral solution at 4.28 mg per 1 mL of propranolol hydrochloride oral solution equivalent to 3.75 mg of propranolol per 1 mL in a pre-filled syringe. The prefilled syringe provides either 1.0 mg, 1.25 mg, 1.5 mg, 2 mg/, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg or 19 mg of propranolol. The inactive ingredients in propranolol hydrochloride oral solution are strawberry/vanilla flavorings, hydroxyethylcellulose, saccharin sodium, citric acid monohydrate, and water.

Alternatively, for P2 study consider use of oral suspension at 0.2 mg per 1 mL in single dose pre-filled syringes.

Pre-filled syringes contain sirolimus in oral solution of 0.05 mg in 0.25 mL solution, 0.08 mg in 0.4 mL, 0.1 mg in 0.5 mL, 0.15 mg in 0.75 mL, 0.2 mg in 1 mL, 0.25 mg in 1.25 mL, 0.3 mg in 1.5 mL, 0.4 mg in 2 mL, 0.5 mg in 2.5 mL, 0.6 mg in 3 mL, 0.8 mg in 4 mL, 1.0 mg in 5 mL. The inactive ingredients in bMED sirlomus oral solution are same as sirlomus oral solution at 20% sirolimus concentration. The inactive ingredients in sirolimus oral solution are PHOSAL 50 PG™ (phosphatidylcholine, propylene glycol, mono- and di-glycerides, ethanol, soy fatty acids, and ascorbyl palmitate) and polysorbate 80. RAPAMUNE™ Oral Solution contains 1.5%-2.5% ethanol.

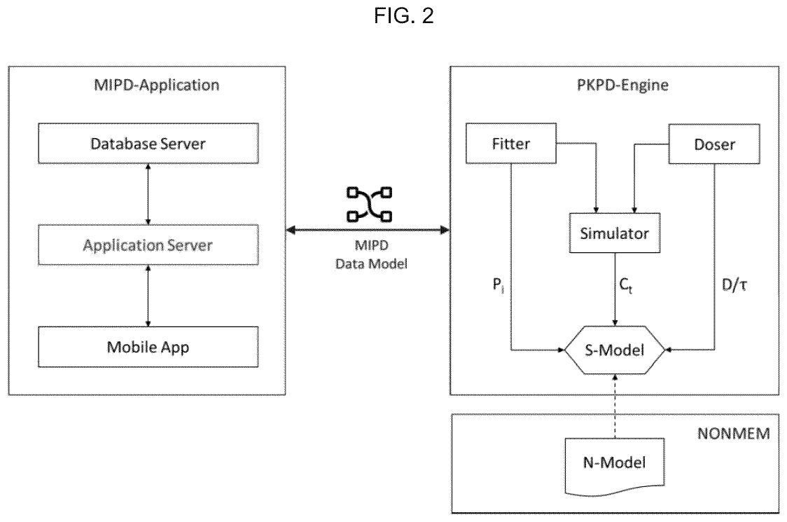

In alternative embodiments, precision dosing (MIPD) used to practice methods as provided herein comprise methods as in FIG. 2, which illustrates an architecture of an exemplary MIPD system. The MIPD business logic is contained in a PKPD-engine, which is implemented as a service that can be called by the main application. The MIPD service and application can communicate by exchanging a specific MIPD data object.

Patients being treated for infantile hemangioma receive a 1 mg/kg oral dose of propranolol, followed by sparse PK sampling with three samples collected at three time points over 4 hours, and optionally at approximately 1, 2 and 4 hours after propranolol dose administration. Propranolol concentrations are incorporated into a population PK model to generate a patient specific PK-model and an optimal, patient-specific starting dose at the predicted minimum starting dose. Following initiation of propranolol at this PK-guided dose, dose escalation occur on weekly interval to a target optimal effective dose. After, each dose escalation, at least one sample is collected and incorporated in the patient-specific model to generate an updated patient specific PK model, a patient-specific next dose to achieve the predicted next target blood or plasma propranolol concentration. This procedure is continued until the patient achieves the predicted optimal target blood or plasma propranolol concentration. In the event of dose limiting toxifies, the dose is reduced to the preceding weeks tolerable dose.

There-after, on a monthly basis, at least one sample is collected and incorporated in the patient-specific model to generate an updated patient specific PK model, and a patient-specific dose to achieve the predicted optimal target blood or plasma propranolol concentration.

### Example 3: Exemplary Method for Treating Lambert Eaton Myasthenic Syndrome

This example describes an exemplary method for treating Lambert Eaton Myasthenic Syndrome.

Doses should be individualized based on Area Under the Curve (AUC) 0 to 4 hours of blood or plasma amifampridine concentration and use of calculation of the amifampridine dose using the trapezoidal rule or use of a Bayesian Model Informed pharmacokinetic model (noncompartmental PK analysis can use estimates of total drug exposure; and total drug exposure can be estimated by area under the curve (AUC) methods, for example, using the trapezoidal rule (numerical integration)). In alternative embodiments, the_Lambert Eaton Myasthenic Syndrome (LEMS) treatment comprise:
Treatment of adult and pediatric patient 5 years of age and older with Lambert Eaton Myasthenic Syndrome (LEMS)
Starting test dose for the dose escalation phase is 0.1 mg/kg three times daily.
After 1 or more days adjust dose three or four times daily to a target of between about 20 ng/mL*h to 30 ng/mL*h AUC₀₋₄ₕ blood or plasma amifampridine after the PM dose;
After 4 or more days can adjust dose three or four times daily to a target of between about 30 ng/mL*h to 40 ng/mL*h AUC₀₋₄ₕ blood or plasma amifampridine after the PM dose;
After 1 or more weeks days can adjust dose three or four times daily to a target of between about 40 ng/mL*h to 50 ng/mL*h AUC₀₋₄ₕ blood or plasma amifampridine after the PM dose;
After 10 or more days can adjust dose three or four times daily to a target of between about 50 ng/mL*h to 60 ng/mL*h AUC₀₋₄ₕ blood or plasma amifampridine after the PM dose;
After 2 or more weeks can adjust dose three or four times daily to a target of between about 60 ng/mL*h to 75 ng/mL*h AUC₀₋₄ₕ blood or plasma amifampridine after the PM dose;
After 2½ or more weeks can adjust dose three or four times daily to a target of between about 75 ng/mL*h to 90 ng/mL*h AUC₀₋₄ₕ blood or plasma amifampridine after the PM dose;
After 3 or more weeks can adjust dose three or four times daily to a target of between about 90 ng/mL*h to 105 ng/mL*h AUC₀₋₄ₕ blood or plasma amifampridine after the PM dose;
After 3½ or more weeks days can adjust dose three or four times daily to a target of between about 105 ng/mL*h to 120 ng/mL*h AUC₀₋₄ₕ blood or plasma amifampridine after the PM dose;
After 4 or more weeks can adjust dose three or four times daily to a target of between about 120 ng/mL*h to 135 ng/mL*h AUC₀₋₄ₕ blood or plasma amifampridine after the PM dose;
After 4½ or more weeks can adjust dose three or four times daily to a target of between about 135 ng/mL*h to 150 ng/mL*h AUC₀₋₄ₕ blood or plasma amifampridine after the PM dose;
After completing dose escalation phase, readjust the dose monthly to the highest of AUC₀₋₄ₕ blood or plasma amifampridine concentration achieved during the during the dose escalation period;
Amifampridine can cause seizures. Consider discontinuation or dose-reduction of amifampridine in patients who have a seizure while on treatment.
   In alternative embodiments, the dose and blood sample collection timing for trough blood concentration determination or for AUC blood concentration determination comprise:

Calculation of AUC₀₋₄ₕ blood or plasma drug concentration is dependent on the amount and timing of prior drug administration, the timing of blood sample collection, the blood collection device, the laboratory assay method and an individualized and adaptive pharmacokinetic model of drug clearance.

After the test dose, three blood sample collections should occur at about one, two about four hours afterward the first test dose administration.

After, each dose escalation, one to three blood samples are collected and incorporated in the patient-specific model to generate an updated patient specific PK model, a patient-specific next dose to achieve the predicted next target blood or plasma amifampridine concentration.

A single sample is collected at about three hours after the PM administration, two samples are collected immediately prior to the PM dose administration and about three hours afterwards, or three samples are collected immediately prior to PM dose administration and about 2 hours and 4 hours afterwards. This procedure is continued until the patient achieves the predicted optimal target blood or plasma amifampridine concentration.

In the event of dose limiting toxifies, the dose is reduced to the preceding tolerable dose.

There-after, on a monthly basis, at least one sample is collected and incorporated in the patient-specific model to generate an updated patient specific PK model, and a patient-specific dose to achieve the predicted optimal target blood or plasma amifampridine concentration.

Each blood sample should occur immediately after the blood collection device is removed from the blood collection blister card.

The blood samples should be placed in the blood sample pouch.

The pouch should be placed in a shipping box and sent to the designated assay laboratory for the determination of blood amifampridine concentrations.

Drug administration should occur immediately after the amifampridine capsule is removed from the capsule blister card (that has a seven-day supply of capsules).

Any missed dose should not be taken, instead, the patient should use the next specified dose on the blister card.

After seven days, the empty card (or partial empty blister card, in the event of a missed dose) should be placed into a shipping package and sent to the designated assay laboratory for determination of dose timing and adherence.

### Calculation of Amifampridine Dose Using the Linear Trapezoidal Rule:

Adjusted dose (mg)=Actual Dose (mg)×Target AUC0-4h (mg/mL*h)/Actual AUC0-4h (mg/mL*h) using the linear trapezoidal rule.

### Calculation of Blood or Plasma Amifampridine Concentration Calculation Using Bayesian Model Informed Pharmacokinetic Model

In alternative embodiments, a pharmacokinetic (PK) model is used to approximate pharmacokinetic (PK) profiles of patients. For instance, current methods to determine a patient-specific pharmacokinetic profile for amifampridine include performing multiple blood tests. After (test dose, minimum therapeutic dose) of amifampridine is administered, six or more blood draws are performed over 4-hour post-administration period. As can be appreciated, such a procedure is especially taxing on a patient, healthcare provider, and lab because of the numerous separate blood draws.

To address this problem, the exemplary model generator as provided herein is configured to generate relatively accurate pharmacokinetic models based upon a sample of patients with varying body weights, height, renal function, liver function, age, genders, pharmacogenetic polymorphism and concomitant medications. These models are then used to determine or approximate a pharmacokinetic profile of a patient without having to subject a patient to all the blood draws and subsequent analysis.

In alternative embodiments, PK models are determined using patient samples selected from one or more sets of patient data. The patient samples may be, for example, selected among patients who have already been subscribed a therapeutic dosing regimen using the above-described blood draw procedure. The patient samples may also include patients specifically selected to go through the blood draw procedure for the purpose of creating the models. The patient samples may include patients from one hospital or medical system and/or patients associated from multiple hospitals, medical systems, geographic regions, etc.

In alternative embodiments, patient samples include data for patients of varying body weights, height, renal function, liver function, age, genders, pharmacogenetic polymorphism and concomitant drug therapies. In some embodiments, the data for the patients may be separated into body weight and renal function brackets such that a separate model is generated for each bracket. The patient data may additionally or alternatively be partitioned based on pharmacogenetic polymorphism.

In alternative embodiments, post-drug administration (for example, post-amifampridine administration) blood samples are collected from each patient after certain durations of time. In alternative embodiments, the blood samples are collected at different times and/or the number of blood samples collected are fewer or greater. For instance, fewer blood samples may be collected after the initial test dose.

In alternative embodiments, the exemplary model generator creates a PK patient model by performing a Bayesian analysis that uses previous knowledge of blood or plasma amifampridine concentration in the sampled patients over time after administration of the (test dose, minimum therapeutic dose). In some instances, the model generator is configured to analyze each patient's sampled dosing history in conjunction with pre-administration blood or plasma amifampridine concentration levels, so that washout data is not needed to construct the PK models. In other embodiments, the model generator uses patient washout data in conjunction with the post administration blood or plasma amifampridine concentration levels to create one or more pharmacokinetic models. Patient washout data corresponds to a baseline where the patient does not include propranolol in their system.

In alternative embodiments, the exemplary model generator creates the one or more PK models using, for example, the patient sample data. The model generator may combine the individual patient samples into one or more population profiles (for example, weight sets, age sets, renal function, liver function, pharmacogenetic polymorphisms, etc.), which is then used as a basis for the respective pharmacokinetic model. In alternative embodiments, the model generator groups the patient samples for different weight, renal function, ages, and/or pharmacogenetic polymorphisms into different sets. In alternative embodiments, the model generator then performs covariate and statistical modeling on the grouped patient samples of each set to create a population pharmacokinetic model for that set, for example, as described by Thakker et al. (2017) “Population Pharmacokinetics/Pharmacodynamics of 3,4-Diaminopyridine Free Base in Patients With Lambert-Eaton Myasthenia”, CPT Pharmacometrics Syst. Pharmacol. 2017, 6, pg 625-634, the entirety of which is incorporated herein by reference. In alternative embodiments the model generator uses a model and sampled data from other Bayesian analysis techniques (for example, a naïve Bayes classifier).

In this exemplary embodiment, the covariate model used by the model generator determines relationships between pharmacokinetic parameters (for example, how quickly amifampridine is metabolized, etc.) and patient characteristics (for example, body weight, clinical laboratory data, age, etc.). In alternative embodiments, the model generator uses a statistical model to determine variance in pharmacokinetic parameters among the sampled patients in addition to residual variance because of biological variability between patients, measurement errors, and errors within the fit of the sampled data to the pharmacokinetic model.

In alternative embodiments, the exemplary model generator is configured to perform the covariate and statistical modeling using the nonlinear mixed effects modeling software NONMEM version 7.3, is used (ICON Development Solutions, Ellicott City, MD). The first-order conditional estimation method with interaction is implemented for all model runs. Run management is performed using Pirana version 2.9.2. Visual predictive checks (VPCs) and bootstrapping is performed using Perl-speaks-NONMEM version 3.7.6. Data manipulation and visualization is performed using the software R version 3.2.0 (R Foundation for Statistical Computing, Vienna, Austria) and RStudio version 0.99.442 (Rstudio, Boston, MA); with the lattice, xpose4, and ggplot2 packages is used for the latter.

The ability of the final population pharmacokinetic model to describe observed concentration data is evaluated by visual predictive check. A total of 1000 simulations is performed with the final population pharmacokinetic model, and the 90% prediction interval (PI) was calculated and used to overlay the observed data for visual predictive check.

In alternative embodiments, a two-compartment model is used for pharmacokinetic modeling. In the PK analysis, 3,4-DAP and 3-Ac DAP concentrations is modeled simultaneously. One and two-compartment models are tested for both the parent and metabolite. The structural model is selected based on an assessment of the objective function value (OFV), precision of parameter estimates, diagnostic plots, and model stability. Given that 3,4-DAP is largely converted to 3-Ac DAP, the fraction of parent converted to metabolite (Fₘ) is fixed to 1 to obtain an identifiable model. Thus, all clearance and volume parameters for 3-Ac DAP are relative to the Fₘ and the bioavailability of 3,4-DAP (F), which is summarized as F_{3ACDAP} (the product of Fₘ and F). Once the PK model met acceptance criteria, the individual PK estimates is fixed.

Covariates are analyzed for inclusion in the PK components of the model. Visual inspection is performed using box and scatter plots (for categorical and continuous covariates, respectively) of the individual deviations from typical population values in the PK against covariates. In the population PK model, the following covariates are explored: total body weight (TBW), serum creatinine (SCR), age, and gender. A forward inclusion (P<0.05 and DOFV >3.8) and backward elimination (P<0.01 and DOFV >6.6) approach are used to evaluate statistical significance for inclusion of covariates in the model. Descriptive statistics of the individual empirical Bayesian estimates (EBEs) is calculated.

The relationship between TBW and PK parameters specifically is evaluated using an allometric relationship for clearance parameters (systemic clearance (CL/F), intercompartmental clearance (Q/F), and metabolite clearance (CLₘ/F_{3ACDAP})) and a linear scale for volume parameters (V/F for one-compartment model; central compartment volume (V_{C}/F) and peripheral compartment volume for 2-compartment model. Fixed exponents of 0.75 and 1 for clearance and volume parameters, respectively, were applied:

$${Wt}_{i} 0 : 75$$

$$CL = F 5 {CL}_{std}$$

where CL_{std} and V_{std} denote the population estimates of CL/F and V/F, respectively, WTₘ denotes the median TBW of the evaluated patients, and Wtᵢ represents the TBW for the ith patient. Other continuous and categorical covariates were tested using a power model and centered using the median covariate value for the sample.

In alternative embodiments, responsive to creating one or more pharmacokinetic models, the exemplary model generator provides the pharmacokinetic model(s) to the PK server. The transmission may be over a private network, such as a local area network, or over a public network, such as an Internet. The model generator may also store the models to the database, which is also accessible by the PK server via one or more interfaces. In other instances, the model generator may be integrated with the PK server.

In alternative embodiments, the exemplary model generator refines the models for each patient. For instance, the PK server may receive patient specific information including, weight, serum creatinine, and dosing level for previous treatments. The model generator uses the previous treatment information (for example, dosing amounts, intervals, etc.) to refine or adjust the model such that dosing recommendations and a pharmacokinetic profile are more aligned to the specific patient but still account for potential patient variance. The model generator transmits the patient-specific model to the PK server.

In alternative embodiments, the PK server is configured to create patient-specific models using the pharmacokinetic model provided by the model generator to account for the patient-specific pharmacokinetic variance. In this manner, one or more base models are refined or adjusted by the PK server responsive to receiving previous treatment information for a specific patient. The PK server may be configured to store the patient-specific model to the database for subsequent uses by the same healthcare provider or other healthcare providers.

In alternative embodiments, once a PK profile for a patient is generated, the PK server is configured to transmit the PK profile to the drug monitoring tool. In some embodiments, the PK server can encrypt the data file prior to transmission. The encryption can be specific to a particular patient such that the drug monitoring tool can only open and process a received PK profile if the tool has a patient specific authentication key.

In alternative embodiments, methods as provided herein use a drug monitoring tool as described by U.S. Pat. No. 10,896,749, which comprises a data receiver and an interactive user interface, where the data receiver is configured to receive a pharmacokinetic (PK) profile of a patient, and an interactive user interface is configured to display a time-varying therapeutic drug or plasma protein level of the patient. The time-varying therapeutic drug or plasma protein level is based on an administered dose of a drug, and the PK profile of the patient.

In alternative embodiments, the drug monitoring tool used in methods as provided herein comprises: a data receiver configured to receive, from a secured server, a patient pharmacokinetic (PK) profile of a patient, wherein the secured server comprises: (1) a Bayesian model of pharmacokinetic (PK) profiles of sampled patients, the Bayesian model including (i) a blood or plasma drug clearance and (ii) a volume of distribution relationship for a plasma protein or drug based upon at least one of patient body weight and rum creatinine, and (2) a PK server configured to determine the patient PK profile based upon the Bayesian model, and at least one of a bodyweight and serum creatinine of the patient; and an interactive user interface configured to: display to the patient a graphical representation predictive of a time-varying drug level in the patient, the time-varying drug level determined by the drug monitoring tool based on the timing and amount of an administered dose of the drug to the patient and the patient PK profile.

### Dosage Forms and Strengths

In alternative embodiments, bMED amifampridine is available for oral administration in the following dosage forms and strengths and supplied on a blister card that time-stamps the use of the single dose pill, tablet or capsule, or the blood collection device.

In alternative embodiments, the sachets, tablets, pills or capsules for administration provide either 2 mg, 2.5 mg, 3 mg, 3.5 mg, 3 mg, 4 mg, 5 mg, 6 mg, 8 mg, 10 mg, 12 mg, 14 mg, 17 mg, 20 mg, 23 mg, 26 mg, 30 mg, 45 mg, 40 mg, 45 mg, or 50 mg of amifampridine.

In alternative embodiments, treating Lambert Eaton Myasthenic Syndrome with an accelerated dose escalation phase comprises use of an accelerated titration schedule:

In alternative embodiments, doses are individualized based on Area Under the Curve (AUC) of blood or plasma amifampridine concentration and use of a Bayesian Model Informed pharmacokinetic model or calculation of the dose using the trapezoidal rule.

In alternative embodiments of an accelerated dose escalation phase:
Treatment of adult and pediatric patient 5 years of age and older with Lambert Eaton Myasthenic Syndrome (LEMS)
Starting test dose for the dose escalation phase is 0.1 mg/kg three times daily
After 1 or more days adjust dose three or four times daily to a target of between about 30 ng/mL*h to 45 ng/mL*h AUC₀₋₄ₕ blood or plasma amifampridine after the PM dose;
After 4 or more days can adjust dose three or four times daily to a target of between about 45 ng/mL*h to 60 ng/mL*h AUC₀₋₄ₕ blood or plasma amifampridine after the PM dose;
After 1 or more weeks days can adjust dose three or four times daily to a target of between about 60 ng/mL*h to 80 ng/mL*h AUC₀₋₄ₕ blood or plasma amifampridine after the PM dose;
After 10 or more days can adjust dose three or four times daily to a target of between about 80 ng/mL*h to 100 μg/mL*h AUC₀₋₄ₕ blood or plasma amifampridine after the PM dose;
After 2 or more weeks can adjust dose three or four times daily to a target of between about 100 ng/mL*h to 125 ng/mL*h AUC₀₋₄ₕ blood or plasma amifampridine after the PM dose;
After 2 or more weeks can adjust dose three or four times daily to a target of between about 125 μg/mL*h to 150 ng/mL*h AUC₀₋₄ₕ blood or plasma amifampridine after the PM dose;
Readjust dose monthly after the dose escalation phase to the highest of AUC₀₋₄ₕ blood or plasma amifampridine concentration obtained during the dose escalation phase;
Amifampridine can cause seizures. Consider discontinuation or dose-reduction of amifampridine in patients who have a seizure while on treatment.

In alternative embodiments, treating Duchennes Muscular Dystrophy, and determining drug dosages for treating Duchennes Muschular Dystrophy, comprises:

In alternative embodiments, doses are individualized based on Area Under the Curve (AUC) 0-8 h to of blood or plasma 21-desDFZ (the active metabolite of deflazacort) concentration and calculation of the deflazacort dose using the trapezoidal rule or the use of a Bayesian Model Informed pharmacokinetic model.

In alternative embodiments, the Duchennes Muscular Dystrophy (DMD) treatment of children and adults ages 2+ comprises:

To attain a blood or plasma AUC₀₋₈ₕ target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or 250 ng/mL*h;
Treatment of adult and pediatric patient 2 years of age and older with Duchennes Muscular Dystrophy (DMD)
Starting test dose for the of 0.9 mg/kg daily.
After 1 or more days adjust dose to a target of about 250 ng/mL*h AUC₀₋₈ₕ blood or plasma concentration for 21-desDFZ;
Re-adjust dose until obtain a target of about 250 ng/mL*h AUC₀₋₈ₕ blood or plasma concentration for 21-desDFZ for two consecutive measurements,
   Readjust the dose monthly to maintain the target AUC₀₋₈ₕ blood or plasma concentration for 21-desDFZ.

### In Alternative Embodiments, Treatments of Duchenne Muscular Dystrophy (DMD) Comprise Use of Dose Titration Phase to a Tolerable Dose:

Treatment of children and adults ages 1+ years.

To attain a tolerable blood or plasma AUC₀₋₈ₕ target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or about 250 ng/mL*h or about 175 ng/mL*h or about 125 ng/mL*h or about 75 ng/mL*h;
Starting test dose of deflazacort for the titration phase is 0.9 mg/kg daily
After 1 or more days adjust dose daily to a target of between about 225 ng/mL*h to 275 ng/mL*h AUC₀₋₈ₕ blood or plasma 21-desDFZ concentration after the AM dose;
After 1 month or more months can adjust dose to a target of between about 150 ng/mL*h to 200 ng/mL*h AUC₀₋₈ₕ blood or plasma 21-desDFZ concentration after the AM dose;
After 2 months or more months can adjust dose daily to a target of between about 100 ng/mL*h to 135 ng/mL*h AUC₀₋₈ₕ blood or plasma 21-desDFZ concentration after the AM dose;
After 3 or more months can adjust dose daily to a target of between about 65 ng/mL*h to 85 μg/mL*h AUC₀₋₈ₕ blood or plasma 21-desDFZ concentation after the AM dose;
Readjust the dose monthly to maintain the tolerable target AUC₀₋₈ₕ blood or plasma concentration for 21-desDFZ;

In alternative embodiments, the treatments of Duchenne Muscular Dystrophy with the combination of deflazacort and ataluren comprise:

In alternative embodiments, doses of deflazacort are individualized based on Area Under the Curve (AUC) 0-8 h to of blood or plasma 21-desDFZ (the active metabolite of deflazacort) concentration and calculation of the deflazacort dose using the trapezoidal rule or the use of a Bayesian Model Informed pharmacokinetic model. Deflazacort should be titrated to a tolerable blood or plasma concentration of 21-desDFZ. After obtaining a tolerable dose of deflazacort, doses of ataluren should be individualized based on C₆ₕ blood or plasma ataluren concentration after the midday dose or trough blood or plasma ataluren concentration prior to the evening dose or use of a Bayesian Model Informed pharmacokinetic model.

In alternative embodiments, a Duchenne Muscular Dystrophy (DMD) treatment comprises:
After obtaining a tolerable dose of deflazacort, initiate treatment of children and adults at ages 1+ years.
Starting test dose of ataluren is 10 mg/kg body weight in the morning, 10 mg/kg body weight midday and 20 mg/kg body weight in the evening (for total daily dose of 40 mg/kg body weight),
After 1 week adjust the dose, proportionally, three times daily to a target of between about 5 μg/mL to 10 μg/mL C₆ₕ blood or plasma ataluren concentration after the midday dose or trough blood or plasma ataluren concentration prior to the evening dose.
Readjust the dose monthly to maintain the target of between about 5 μg/mL to 10 μg/mL C₆ₕ blood or plasma ataluren concentration after the midday dose or trough blood or plasma ataluren concentration prior to the evening dose.
   In alternative embodiments, the dose and blood sample collection timing for AUC Blood Concentration Determination comprises:

Calculation of AUC₀₋₈ₕ blood or plasma 21-desDFZ concentration is dependent on the amount and timing of prior drug administration, the timing of blood sample collection, the blood collection device, the laboratory assay method and an individualized and adaptive pharmacokinetic model of drug clearance.

After the starting dose, three blood sample collections should occur at about one, three about eight hours afterward the starting dose administration. In alternative embodiments, four blood sample collections should occur at about one, two, four and twelve hours afterward the starting dose administration.

Each week or month thereafter, three blood samples are collected and used to calculate the adjusted using the trapezoidal rule the incorporated in thepatient-specific model to generate an updated patient specific PK model, a patient-specific next dose to achieve the predicted next target blood or plasma amifampridine concentration.

In the event of dose limiting toxifies, the dose is reduced by 20% until a tolerable dose is achieved.

There-after, on a monthly basis, at least one sample is collected and incorporated in the patient-specific model to generate an updated patient specific PK model, and a patient-specific dose to achieve the predicted optimal target plasma21-desDFZ concentration.

Each blood sample should occur immediately after the blood collection device is removed from the blood collection blister card.

The blood samples should be placed in the blood sample pouch.

The pouch should be placed in a shipping box and sent to the designated assay laboratory for the determination of blood amifampridine concentrations.

Drug administration should occur immediately after the deflazacort capsule is removed from the capsule blister card (that has a seven-day supply of capsules).

Any missed dose should not be taken, instead, the patient should use the next specified dose on the blister card.

After seven days, the empty card (or partial empty blister card, in the event of a missed dose) should be placed into a shipping package and sent to the designated assay laboratory for determination of dose timing and adherence.

### Calculation of Deflazacort Dose Using the Linear Trapezoidal Rule

In alternative embodiments, the adjusted dose (mg) is calculated using the linear trapezoidal rules as follows:

Adjusted dose (mg) will equal the closest available dose to the Actual Dose (mg)×Target AUC₀₋₄ₕ (mg/mL*h)/Actual AUC₀₋₄ₕ (mg/mL*h) using the linear trapezoidal rule. In alternative embodiments, adjusted dose (mg) will equal the closest available dose to the Actual Dose (mg)×Target AUC₀₋₈ₕ (mg/mL*h)/Actual AUC₀₋₈ₕ (mg/mL*h) using the linear trapezoidal rule.

In alternative embodiments a software tool will calculate the adjusted dose will equal the closest available dose to the Actual Dose (mg)×Target AUC₀₋₄ₕ (mg/mL*h)/Actual AUC₀₋₄ₕ (mg/mL*h) using the linear trapezoidal rule. In alternative embodiments, that adjusted dose (mg)=closest available dose to the Actual Dose (mg)×Target AUC₀₋₈ₕ (mg/mL*h)/average of the Actual AUC₀₋₈ₕ (mg/mL*h) using the linear trapezoidal rule for the current and two previous months.

### Calculation of Deflazacort Dose Using the Logarithmic Trapezoidal Rule

In alternative embodiments, the adjusted dose (mg) is calculated using the logarithmic trapezoidal rules as follows:

Adjusted dose (mg) will equal the closest available dose to the Actual Dose (mg)×Target AUC₀₋₈ₕ (mg/mL*h)/Actual AUC₀₋₈ₕ (mg/mL*h) using the logarithmic trapezoidal rule. In alternative embodiments, adjusted dose (mg) will equal the closest available dose to the Actual Dose (mg)×Target AUC₀₋₁₂ₕ (mg/mL*h)/Actual AUC₀₋₁₂ₕ (mg/mL*h) using the logarithmic trapezoidal rule.

In alternative embodiments a software tool will calculate the adjusted dose will equal the closest available dose to the Actual Dose (mg)×Target AUC₀₋₈ₕ (mg/mL*h)/Actual AUC₀₋₈ₕ (mg/mL*h) using the logarithmic trapezoidal rule. In alternative embodiments, that adjusted dose (mg)=closest available dose to the Actual Dose (mg)×Target AUC₀₋₁₂ₕ (mg/mL*h)/average of the Actual AUC₀₋₁₂ₕ (mg/mL*h) using the logarithmic trapezoidal rule for the current and two previous months.

In alternative embodiments, the calculation of Blood or Plasma 21-desDFZ uses Bayesian Model Informed Pharmacokinetic Model:

In alternative embodiments, a pharmacokinetic (PK) model is used to approximate pharmacokinetic (PK) profiles of patients. For instance, current methods to determine a patient-specific pharmacokinetic profile for deflazacort include performing multiple blood tests. After (test dose, minimum therapeutic dose) of amifampridine is administered, six or more blood draws are performed over 4-hour post-administration period. As can be appreciated, such a procedure is especially taxing on a patient, healthcare provider, and lab because of the numerous separate blood draws.

To address this problem, the exemplary model generator as provided herein is configured to generate relatively accurate pharmacokinetic models based upon a sample of patients with varying body weights, height, renal function, liver function, age, genders, pharmacogenetic polymorphism and concomitant medications. These models are then used to determine or approximate a pharmacokinetic profile of a patient without having to subject a patient to all the blood draws and subsequent analysis.

In alternative embodiments, PK models are determined using patient samples selected from one or more sets of patient data. The patient samples may be, for example, selected among patients who have already been subscribed a therapeutic dosing regimen using the above-described blood draw procedure. The patient samples may also include patients specifically selected to go through the blood draw procedure for the purpose of creating the models. The patient samples may include patients from one hospital or medical system and/or patients associated from multiple hospitals, medical systems, geographic regions, etc.

In alternative embodiments, patient samples include data for patients of varying body weights, height, renal function, liver function, age, genders, pharmacogenetic polymorphism and concomitant drug therapies. In some embodiments, the data for the patients may be separated into body weight and renal function brackets such that a separate model is generated for each bracket. The patient data may additionally or alternatively be partitioned based on pharmacogenetic polymorphism.

In alternative embodiments, post-drug administration (for example, post-deflazacort administration) blood samples are collected from each patient after certain durations of time. In alternative embodiments, the blood samples are collected at different times and/or the number of blood samples collected are fewer or greater. For instance, fewer blood samples may be collected after the initial test dose.

In alternative embodiments, the exemplary model generator creates a PK patient model by performing a Bayesian analysis that uses previous knowledge of blood or plasma 21-desDFZ concentration in the sampled patients over time after administration of the (test dose, minimum therapeutic dose). In some instances, the model generator is configured to analyze each patient's sampled dosing history in conjunction with pre-administration blood or plasma 21-desDFZ concentration levels, so that washout data is not needed to construct the PK models. In other embodiments, the model generator uses patient washout data in conjunction with the post administration blood or plasma amifampridine concentration levels to create one or more pharmacokinetic models. Patient washout data corresponds to a baseline where the patient does not include 21-desDFZ in their system.

In alternative embodiments, the exemplary model generator creates the one or more PK models using, for example, the patient sample data. The model generator may combine the individual patient samples into one or more population profiles (for example, weight sets, age sets, renal function, liver function, pharmacogenetic polymorphisms, etc.), which is then used as a basis for the respective pharmacokinetic model. In alternative embodiments, the model generator groups the patient samples for different weight, renal function, ages, and/or pharmacogenetic polymorphisms into different sets. In alternative embodiments, the model generator then performs covariate and statistical modeling on the grouped patient samples of each set to create a population pharmacokinetic model for that set, for example, as described by Thakker et al. (2017) “Population Pharmacokinetics/Pharmacodynamics of 3,4-Diaminopyridine Free Base in Patients With Lambert-Eaton Myasthenia”, CPT Pharmacometrics Syst. Pharmacol. 2017, 6, pg 625-634, the entirety of which is incorporated herein by reference. In alternative embodiments the model generator uses a model and sampled data from other Bayesian analysis techniques (for example, a naïve Bayes classifier).

In this exemplary embodiment, the covariate model used by the model generator determines relationships between pharmacokinetic parameters (for example, how quickly 21-desDFZ is metabolized, etc.) and patient characteristics (for example, body weight, clinical laboratory data, age, etc.). In alternative embodiments, the model generator uses a statistical model to determine variance in pharmacokinetic parameters among the sampled patients in addition to residual variance because of biological variability between patients, measurement errors, and errors within the fit of the sampled data to the pharmacokinetic model.

In alternative embodiments, the exemplary model generator is configured to perform the covariate and statistical modeling using the nonlinear mixed effects modeling software NONMEM version 7.3, is used (ICON Development Solutions, Ellicott City, MD). The first-order conditional estimation method with interaction is implemented for all model runs. Run management is performed using Pirana version 2.9.2. Visual predictive checks (VPCs) and bootstrapping is performed using Perl-speaks-NONMEM version 3.7.6. Data manipulation and visualization is performed using the software R version 3.2.0 (R Foundation for Statistical Computing, Vienna, Austria) and RStudio version 0.99.442 (RStudio, Boston, MA); with the lattice, xpose4, and ggplot2 packages is used for the latter.

In alternative embodiments, the ability of the final population pharmacokinetic model to describe observed concentration data is evaluated by visual predictive check. A total of 1000 simulations is performed with the final population pharmacokinetic model, and the 90% prediction interval (PI) was calculated and used to overlay the observed data for visual predictive check.

In alternative embodiments, a two-compartment model is used for pharmacokinetic modeling. In the PK analysis, 21-desDFZ concentrations is modeled simultaneously. One and two-compartment models are tested for both the parent and metabolite. The structural model is selected based on an assessment of the objective function value (OFV), precision of parameter estimates, diagnostic plots, and model stability. Once the PK model met acceptance criteria, the individual PK estimates is fixed.

In alternative embodiments, the covariates are analyzed for inclusion in the PK components of the model. Visual inspection is performed using box and scatter plots (for categorical and continuous covariates, respectively) of the individual deviations from typical population values in the PK against covariates. In the population PK model, the following covariates are explored: total body weight (TBW), serum creatinine (SCR), age, and gender. A forward inclusion (P<0.05 and DOFV >3.8) and backward elimination (P<0.01 and DOFV >6.6) approach are used to evaluate statistical significance for inclusion of covariates in the model. Descriptive statistics of the individual empirical Bayesian estimates (EBEs) is calculated.

In alternative embodiments, the relationship between TBW and PK parameters specifically is evaluated using an allometric relationship for clearance parameters (systemic clearance (CL/F), intercompartmental clearance (Q/F), and metabolite clearance (CLₘ/F_{3ACDAP})) and a linear scale for volume parameters (V/F for one-compartment model; central compartment volume (V_{c}/F) and peripheral compartment volume for 2-compartment model. Fixed exponents of 0.75 and 1 for clearance and volume parameters, respectively, were applied:

$${Wt}_{i} 0 : 75$$

$$CL = F 5 {CL}_{std}$$

where CL_{std} and V_{std} denote the population estimates of CL/F and V/F, respectively, WTₘ denotes the median TBW of the evaluated patients, and WTᵢ represents the TBW for the ith patient. Other continuous and categorical covariates were tested using a power model and centered using the median covariate value for the sample.

In alternative embodiments, responsive to creating one or more pharmacokinetic models, the exemplary model generator provides the pharmacokinetic model(s) to the PK server. The transmission may be over a private network, such as a local area network, or over a public network, such as an Internet. The model generator may also store the models to the database, which is also accessible by the PK server via one or more interfaces. In other instances, the model generator may be integrated with the PK server.

In alternative embodiments, the exemplary model generator refines the models for each patient. For instance, the PK server may receive patient specific information including, weight, serum creatinine, and dosing level for previous treatments. The model generator uses the previous treatment information (for example, dosing amounts, intervals, etc.) to refine or adjust the model such that dosing recommendations and a pharmacokinetic profile are more aligned to the specific patient but still account for potential patient variance. The model generator transmits the patient-specific model to the PK server.

In alternative embodiments, the PK server is configured to create patient-specific models using the pharmacokinetic model provided by the model generator to account for the patient-specific pharmacokinetic variance. In this manner, one or more base models are refined or adjusted by the PK server responsive to receiving previous treatment information for a specific patient. The PK server may be configured to store the patient-specific model to the database for subsequent uses by the same healthcare provider or other healthcare providers.

In alternative embodiments, once a PK profile for a patient is generated, the PK server is configured to transmit the PK profile to the drug monitoring tool. In some embodiments, the PK server can encrypt the data file prior to transmission. The encryption can be specific to a particular patient such that the drug monitoring tool can only open and process a received PK profile if the tool has a patient specific authentication key.

In alternative embodiments, methods as provided herein use a drug monitoring tool as described by U.S. Pat. No. 10,896,749, which comprises a data receiver and an interactive user interface, where the data receiver is configured to receive a pharmacokinetic (PK) profile of a patient, and an interactive user interface is configured to display a time-varying 21-desDFZ level of the patient. The time-varying therapeutic 21-desDFZ level is based on an administered dose of a drug, and the PK profile of the patient.

In alternative embodiments, the drug monitoring tool used in methods as provided herein comprises: a data receiver configured to receive, from a secured server, a patient pharmacokinetic (PK) profile of a patient, wherein the secured server comprises: (1) a Bayesian model of pharmacokinetic (PK) profiles of sampled patients, the Bayesian model including (i) a blood or plasma 21-desDFZ clearance and (ii) a volume of distribution relationship for a blood or plasma drug based upon at least one of patient body weight and rum creatinine, and (2) a PK server configured to determine the patient PK profile based upon the Bayesian model, and at least one of a bodyweight and serum creatinine of the patient; and an interactive user interface configured to: display to the patient a graphical representation predictive of a time-varying drug level in the patient, the time-varying drug level determined by the drug monitoring tool based on the timing and amount of an administered dose of the drug to the patient and the patient PK profile.

### Dosage Forms and Strengths

In alternative embodiments, bMED deflazacort is available for oral administration in the following dosage forms and strengths and supplied on a blister card that time-stamps the use of the single dose tablet or capsule, or the blood collection device.

In alternative embodiments, the tablets, sachets, pills or capsules for administration provide either 6 mg, 7 mg, 8 mg, 9 mg, 10 mg, 12 mg, 14 mg, 16 mg, 18 mg, 20 mg, 23 mg, 26 mg, 30 mg, 34 mg, 38 mg, 43 mg, 48 mg, 54 mg, 60 mg, 66 mg, 72 mg or 80 mg of deflazacort.

### Dosage Forms and Strengths

In alternative embodiments, bMED ataluren is available for oral administration in the following dosage forms and strengths and supplied on a blister card that timestamps the use of the single dose sachet, pill or capsule, or the blood collection device.

In alternative embodiments a sachet or capsule for administration comprising ataluren comprises unit dosages of about: 50 mg, 60 mg, 70 mg, 85 mg, 105 mg, 125 mg, 150 mg, 180 mg, 210 mg, 250 mg, 260 mg, 310 mg, 375 mg, 450 mg, 525 mg, 650 mg, 775 mg, 925 mg, 1000 mg, 1100 mg, 1350 mg, 1600 mg, 1900 mg or 2300 mg of ataluren granules, optionally for oral suspension.

In alternative embodiments, “Individualized dosing” and “PK-guided dosing”, “precision dosing” are interchangeable terms, mean administering each patient by a drug's PK properties in the patient.

In alternative embodiments, terms of “plasma concentration”, “plasma drug concentration”, “serum concentration”, “serum drug concentration”, “plasma level”, “level” or the terms “blood concentration” “blood drug concentration” or “blood level” in the context of drug concentration, are interchangeable, with the appropriate translation between blood and plasma concentration, and mean drug concentration in humans or animals, and measured by and interpreted by skilled in the art see Loftsson T. Essential Pharmacokinetics—1st Edition. Elsevier. 2015.

In alternative embodiments, “target therapeutic blood or plasma drug concentration”, or a similar term, is within the therapeutic blood or plasma range, optionally about the mid-point of a therapeutic level range of blood or plasma concentration. For example, if the therapeutic concentration, for example, the effective or efficacious blood or plasma concentration range, for treating a disease as provided herein is between 1000 ng/ml to 1400 ng/ml, the target therapeutic blood or plasma drug concentration can be about 1000 ng/ml, about 1300 ng/ml, or optionally the mid-point about 1200 ng/ml.

In alternative embodiments, therapeutic in the context of blood or plasma concentration means effective or efficacious in treating a medical condition(s).

### Products of Manufacture and Kits

Provided are products of manufacture comprising and kits comprising therapeutic drug compositions as provided herein, and/or for practicing methods as provided herein; and optionally, products of manufacture and kits can further comprise instructions for practicing methods as provided herein.

In alternative embodiments kits comprise a companion diagnostic, which optionally comprises a dried blood spot collection device, a volumetric dried blood spot, volumetric absorptive microsampling (VAMS) or an at-home high-volume liquid blood collection device, optionally with a time stamp device operably connected to or associated with the dried blood spot collection device or at-home high-volume liquid blood collection device,
and optionally the volumetric dried blood spot, volumetric absorptive microsampling (VAMS) or the at-home high-volume liquid blood collection device comprises a hand-held device comprising a plurality of microneedles or volumetric needles, wherein the plurality of microneedles or volumetric needle are capable of capturing a predefined volummetric blood sample, for example, a blood sample of between about 5 μl and 500 μl, or between about 10 μl and 100 μl, or about 5 μl, 10 μl, 15 μl, 20 μl, 25 μl, 30 μl or 35 μl, and optionally the volumetric dried blood spot, volumetric absorptive microsampling (VAMS) or the at-home high-volume liquid blood collection device comprises a MITRA™ device (by NEOTERYX™), or a bladeless microneedle array as in a TAPMICRO™ or HALO™ device (yourbio, Medford, MA),
and optionally the dried blood spot collection device comprises a filter paper card for the collection of one or more blood samples (or blood drops, optionally the blood drop is generated by a user or patient using a lancet or a fingerstick), and optionally the filter paper comprises one, two or more designated spaces for a blood drop to be placed, and optionally each filter paper and/or each designated space has a unique bar code or QR code, and optionally the dried blood spot collection device or an at-home high-volume liquid blood collection device comprises one or a plurality of lancets or fingersticks (or fingerprick), and optionally the filter paper is stored and optionally mailed in a gas-impermeable container (optionally a zipper bag), optionally containing one or more desiccant sachets to protect the blood specimens on the filter paper from moisture contamination.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Exemplary Method for Treating Proliferating Infantile Hemangioma

This example describes an exemplary method for treating proliferating infantile hemangioma.

Doses should be individualized based on trough blood or plasma propranolol concentration and use of a Bayesian Model Informed pharmacokinetic model.

### Exemplary Treatment of Proliferating Infantile Hemangioma (IH), or PIH

Initiate treatment at ages 5 weeks to 5 months.
Starting dose is 0.15 mL/kg propranolol hydrochloride oral solution (0.6 mg propranolol/kg) twice daily.
After 3 days adjust dose twice daily to a target of between about 20 ng/mL to 30 ng/mL C₉ₕ or trough blood or plasma propranolol concentration after the AM dose or trough blood or plasma prior to the PM dose;
After 1 week increase dose twice daily to a target of 40 ng/mL to 50 ml C₉ₕ or trough blood or plasma propranolol concentration;
After 2 weeks increase dose twice daily to a target of 60 ng/mL to 80 ng/mLC₉ₕ or trough blood or plasma propranolol concentration;
Readjust dose monthly to a target of 60 ng/mL to 80 ng/mL C₉ₕ or trough blood or plasma propranolol concentration;
Monitor heart rate and blood pressure for about 2 hours after the first dose or increasing dose.

### Maintenance Treatment of Infantile Hemangioma (IH)

Titrate dose to attain a C₉ₕ or trough blood or plasma propranolol concentration of between about 20 ng/mL to 30 ng/mL;
Readjust dose monthly to a target of 20 ng/mL to 30 ng/mL C₉ₕ or trough blood or plasma propranolol concentration.

Consider tapering bMED propranolol after 6 months of maintenance treatment.
Taper bMED propranolol one C₉ₕ or trough blood or plasma propranolol concentration every 2 weeks (to between about 15 ng/mL to 20 ng/mL to between about 10 ng/mL to 15 ng/mL to between about 5 to 10 ng/mL);
If infantile hemangioma (IH) signs (for example, angiopoietin 2, tumor growth) recur during or after the taper of bMED propranolol, consider retreatment.

Evening dose administration should be withheld during fever or infection and resumed upon resolution of the fever or infection.

### Dose and Blood Sample Collection Timing for Trough Blood Concentration Determination

Calculation of trough blood or plasma drug concentration is dependent on the amount and timing of prior drug administration, the timing of acute illness, the timing of blood sample collection, the blood collection device, the laboratory assay method and an individualized and adaptive pharmacokinetic model of drug clearance.

Drug administration should occur immediately after pre-filled syringe is removed from the syringe blister card (that has a seven day supply of pre-filled syringes).

The blood sample collection should occur prior to drug administration of the last syringe on the blister card.

The blood sample should occur immediately after the blood collection device is removed from the blood collection blister card.

The blood sample should be placed in the blood sample pouch.

The pouch, the empty drug blister card and the empty device card should be placed in shipping box and sent to the designated assay laboratory for the determination of blood sirolimus concentrations.

### Calculation of Blood or Plasma Propranolol Calculation Using Bayesian Model Informed Pharmacokinetic Model

In alternative embodiments, a pharmacokinetic (PK) model is used to approximate pharmacokinetic (PK) profiles of patients. For instance, current methods to determine a patient-specific pharmacokinetic profile for infantile hemangioma include performing multiple blood tests. After (test dose, minimum therapeutic dose) of propranolol is administered, five or more blood draws are performed over 9-hour post-administration period. As can be appreciated, such a procedure is especially taxing on a patient, healthcare provider, and lab because of the numerous separate blood draws.

To address this problem, the exemplary model generator as provided herein is configured to generate relatively accurate pharmacokinetic models based upon a sample of patients with varying body weights, postnatal age, postmenstrual age, gestational age, height, renal function, liver function and genders. These models are then used to determine or approximate a pharmacokinetic profile of a patient without having to subject a patient to all of the blood draws and subsequent analysis.

In alternative embodiments, PK models are determined using patient samples selected from one or more sets of patient data. The patient samples may be, for example, selected among patients who have already been subscribed a therapeutic dosing regimen using the above-described blood draw procedure. The patient samples may also include patients specifically selected to go through the blood draw procedure for the purpose of creating the models. The patient samples may include patients from one hospital or medical system and/or patients associated from multiple hospitals, medical systems, geographic regions, etc.

In alternative embodiments, patient samples include data for patients of varying postnatal ages, body weights (or body mass index (“BMI”), medical conditions, clinical laboratory data, and genders. In alternative embodiments, patients' ages vary between postnatal age of 53 to 150 days. In some embodiments, the data for the patients may be separated into age brackets such that a separate model is generated for each bracket. The patient data may additionally or alternatively be partitioned based on weight and/or genders.

In alternative embodiments, post-drug administration (for example, post-propranolol administration) blood samples are collected from each patient after certain durations of time. In alternative embodiments, the blood samples are collected at different times and/or the number of blood samples collected are fewer or greater. For instance, fewer blood samples may be collected from newborns.

In alternative embodiments, the exemplary model generator creates a PK patient model by performing a Bayesian analysis that uses previous knowledge of blood or plasma propranolol concentration in the sampled patients over time after administration of the (test dose, minimum therapeutic dose). In some instances, he model generator is configured to analyze each patient's sampled dosing history in conjunction with pre-administration blood or plasma propranolol concentration levels, so that washout data is not needed to construct the PK models. In other embodiments, the model generator uses patient washout data in conjunction with the post administration blood or plasma propranolol concentration levels to create one or more pharmacokinetic models. Patient washout data corresponds to a baseline where the patient does not include propranolol in their system.

In alternative embodiments, the exemplary model generator creates the one or more PK models using, for example, the patient sample data. The model generator may combine the individual patient samples into one or more population profiles (for example, age sets, weight sets, sex, etc.), which is then used as a basis for the respective pharmacokinetic model. In alternative embodiments, the model generator groups the patient samples for different ages, weights, and/or sex into different sets. In alternative embodiments, the model generator then performs covariate and statistical modeling on the grouped patient samples of each set to create a population pharmacokinetic model for that set, for example, as described by Takechi et al. (2018) “Population Pharmacokinetics and Pharmacodynamics of Oral Propranolol in Pediatric Patients With Infantile Hemangioma”, Clinical Pharmacology, vol 58(10), pg 1361-1370, the entirety of which is incorporated herein by reference. In alternative embodiments the model generator uses a model and sampled data from other Bayesian analysis techniques (for example, a naïve Bayes classifier).

In this exemplary embodiment, the covariate model used by the model generator determines relationships between pharmacokinetic parameters (for example, how quickly propranolol is metabolized, etc.) and patient characteristics (for example, postnatal age, body weight, clinical laboratory data, gender, etc.). In alternative embodiments, the model generator uses a statistical model to determine variance in pharmacokinetic parameters among the sampled patients in addition to residual variance as a result of biological variability between patients, measurement errors, and errors within the fit of the sampled data to the pharmacokinetic model.

In alternative embodiments, the exemplary model generator is configured to perform the covariate and statistical modeling using the nonparametric bootstrap approach with Perl-speaks-NONMEM (PsN) version 4.4.8™ (see for example, Lindbom et al (2004) Computer Methods Programs in Biomed., vol. 75(2), pg 85-94).

The ability of the final population pharmacokinetic model to describe observed concentration data was evaluated by visual predictive check. A total of 1000 simulations were performed with the final population pharmacokinetic model, and the 90% prediction interval (PI) was calculated and used to overlay the observed data for visual predictive check.

In alternative embodiments, a one-compartment model is used for pharmacokinetic modeling. The first-order absorption rate constant (kₐ) is fixed at the value calculated according to the equation tₘₐₓ=Ln(kₐ/kₑₗ)/(kₐ−kₑₗ), using previously reported pharmacokinetic parameters because of a lack of sampling points. Interindividual variability on each parameter assumed a log-normal distribution and was estimated with an exponential error model. An additive error model, a proportional error model, and a combination of additive and proportional error models were investigated to describe the residual variability. A basic model was selected using the Akaike information criterion. Body weight, postnatal age, postmenstrual age, height, serum creatinine, aspartate aminotransferase, alanine aminotransferase, and sex were examined as candidates for pharmacokinetic covariates. As a structural covariate, a model using fixed theoretical exponents (0.75 for CL/F and 1 for apparent central volume of distribution [V/F]) with body weight was included in the basic model to account for the dose calculation (in mg/kg) and growth and development of the infants. The selection of covariates to be included in the model was conducted using stepwise forward selection (P<0.05), followed by backward elimination (P<0.01), based on changes in objective function value (OFV). Continuous covariates were modeled with a power function centered on a median value. A categorical covariate was incorporated as a discrete indicator variable. The adequacy of the constructed population pharmacokinetic model was assessed using goodness-of-fit plots. Goodness-of-fit was investigated using plots of observations versus population prediction (PRED) and individual prediction (IPRED), conditional weighted residuals versus time after the first administration and time after the last administration, conditional weighted residuals versus PRED and absolute individual weighted residuals versus IPRED. Shrinkage in random effects was computed to guide the appropriateness of using ETA (η, empirical Bayes estimate of the interindividual random effect) and IPRED values in the goodness-of-fit assessment.

In alternative embodiments, responsive to creating one or more pharmacokinetic models, the exemplary model generator provides the pharmacokinetic model(s) to the PK server. The transmission may be over a private network, such as a local area network, or over a public network, such as an Internet. The model generator may also store the models to the database, which is also accessible by the PK server via one or more interfaces. In other instances, the model generator may be integrated with the PK server.

In alternative embodiments, the exemplary model generator refines the models for each patient. For instance, the PK server may receive patient specific information including, weight, age, gender, and dosing level for previous treatments. The model generator uses the previous treatment information (for example, dosing amounts, intervals, etc.) to refine or adjust the model such that dosing recommendations and a pharmacokinetic profile are more aligned to the specific patient but still account for potential patient variance. The model generator transmits the patient-specific model to the PK server.

In alternative embodiments, the PK server is configured to create patient-specific models using the pharmacokinetic model provided by the model generator to account for the patient-specific pharmacokinetic variance. In this manner, one or more base models are refined or adjusted by the PK server responsive to receiving previous treatment information for a specific patient. The PK server may be configured to store the patient-specific model to the database for subsequent uses by the same healthcare provider or other healthcare providers.

In alternative embodiments, once a PK profile for a patient is generated, the PK server is configured to transmit the PK profile to the drug monitoring tool. In some embodiments, the PK server can encrypt the data file prior to transmission. The encryption can be specific to a particular patient such that the drug monitoring tool can only open and process a received PK profile if the tool has a patient specific authentication key.

In alternative embodiments, methods as provided herein use a drug monitoring tool as described by U.S. Pat. No. 10,896,749, which comprises a data receiver and an interactive user interface, where the data receiver is configured to receive a pharmacokinetic (PK) profile of a patient, and an interactive user interface is configured to display a time-varying therapeutic drug or plasma protein level of the patient. The time-varying therapeutic drug or plasma protein level is based on an administered dose of a drug, and the PK profile of the patient.

In alternative embodiments, the drug monitoring tool used in methods as provided herein comprises: a data receiver configured to receive, from a secured server, a patient pharmacokinetic (PK) profile of a patient, wherein the secured server comprises: (1) a Bayesian model of pharmacokinetic (PK) profiles of sampled patients, the Bayesian model including (i) a blood or plasma drug clearance and (ii) a volume of distribution relationship for a plasma protein or drug based upon at least one of patient age or body weight, and (2) a PK server configured to determine the patient PK profile based upon the Bayesian model, and at least one of a bodyweight, height, and an age of the patient; and an interactive user interface configured to: display to the patient a graphical representation predictive of a time-varying drug level in the patient, the time-varying drug level determined by the drug monitoring tool based on the timing and amount of an administered dose of the drug to the patient and the patient PK profile.

### Dosage Forms and Strengths

In alternative embodiments, a drug, drug combination or drug formulation as provided herein, for example, comprising bMED Propranolol, is available for oral administration in the following dosage forms and strengths and supplied on a blister card that time-stamps the use of the single dose pre-filled syringe or capsule, or the blood collection device.

In alternative embodiments, a drug, drug combination or drug formulation as provided herein, for example, comprising bMED Propranolol, are formulated as or in granules, for example, as granules for oral suspension. Granules for oral suspension in single dose pre-filled 5 mL syringes, when reconstituted, provide either 0.05 mg/mL, 0.075 mg/mL, 0.1 mg/5 mL, 0.15 mg/5 mL, 0.2 mg/5 mL, 0.25 mg/5 mL, 0.3 mg/5 mL, 0.4 mg/5 mL, 0.5 mg/5 mL, 0.6 mg/5 mL, 0.8 mg/5 mL, 1.Omg/5 mL, 1.25 mg/5 mL, 1.5 mg/5 mL, 2 mg/5 mL, 2.5 mg/5 mL, 3 mg/5 mL, 3.5 mg/5 mL, 4 mg/5 mL, 4.5 mg/5 mL, 5 mg/5 mL, 5.5 mg/5 mL, 6 mg/5 mL, 6.5 mg/5 mL, 7 mg/5 mL, 7.5 mg/5 mL, 8 mg/5 mL, 8.5 mg/5 mL, 9 mg/5 mL, 9.5 mg/5 mL, 10 mg/5 mL, 11 mg/5 mL, 12 mg/5 mL, 13 mg/5 mL, 14 mg/5 mL, 15 mg/5 mL, 16 mg/5 mL, 17 mg/5 mL, 18 mg/5 mL, 20 mg/5 mL, 22 mg/5 mL, 24 mg/5 mL, 26 mg/5 mL, 28 mg/5 mL or 30 mg/5 mL or 2.0 mg/mL of propranolol. The granules have an off white to cream color and are strawberry flavored.

For P2 study consider use of oral solution at 4.28 mg per 1 mL of propranolol hydrochloride oral solution equivalent to 3.75 mg of propranolol per 1 mL in a pre-filled syringe. The prefilled syringe provides either 1.0 mg, 1.25 mg, 1.5 mg, 2 mg/, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg or 19 mg of propranolol. The inactive ingredients in propranolol hydrochloride oral solution are strawberry/vanilla flavorings, hydroxyethylcellulose, saccharin sodium, citric acid monohydrate, and water.

Alternatively, for P2 study consider use of oral suspension at 0.2 mg per 1 mL in single dose pre-filled syringes.

Pre-filled syringes contain sirolimus in oral solution of 0.05 mg in 0.25 mL solution, 0.08 mg in 0.4 mL, 0.1 mg in 0.5 mL, 0.15 mg in 0.75 mL, 0.2 mg in 1 mL, 0.25 mg in 1.25 mL, 0.3 mg in 1.5 mL, 0.4 mg in 2 mL, 0.5 mg in 2.5 mL, 0.6 mg in 3 mL, 0.8 mg in 4 mL, 1.0 mg in 5 mL. The inactive ingredients in bMED sirlomus oral solution are same as sirlomus oral solution at 20% sirolimus concentration. The inactive ingredients in sirolimus oral solution are PHOSAL 50 PG™ (phosphatidylcholine, propylene glycol, mono- and di-glycerides, ethanol, soy fatty acids, and ascorbyl palmitate) and polysorbate 80. RAPAMUNE™ Oral Solution contains 1.5%-2.5% ethanol.

### Example 2: Exemplary Model Informed Precision Dosing (MIPD)

This example describes an exemplary model informed precision dosing (MIPD) used to practice methods as provided herein.

FIG. 2 illustrates an architecture of an exemplary MIPD system. The MIPD business logic is contained in a PKPD-engine, which is implemented as a service that can be called by the main application. The MIPD service and application can communicate by exchanging a specific MIPD data object.

Patients being treated for infantile hemangioma receive a 1 mg/kg oral dose of propranolol, followed by sparse PK sampling with three samples collected at three time points over 4 hours, and optionally at approximately 1, 2 and 4 hours after propranolol dose administration. Propranolol concentrations are incorporated into a population PK model to generate a patient specific PK-model and an optimal, patient-specific starting dose at the predicted minimum starting dose. Following initiation of propranolol at this PK-guided dose, dose escalation occur on weekly interval to a target optimal effective dose. After, each dose escalation, at least one sample is collected and incorporated in the patient-specific model to generate an updated patient specific PK model, a patient-specific next dose to achieve the predicted next target blood or plasma propranolol concentration. This procedure is continued until the patient achieves the predicted optimal target blood or plasma propranolol concentration. In the event of dose limiting toxifies, the dose is reduced to the preceding weeks tolerable dose.

There-after, on a monthly basis, at least one sample is collected and incorporated in the patient-specific model to generate an updated patient specific PK model, and a patient-specific dose to achieve the predicted optimal target blood or plasma propranolol concentration.

### Example 3: Exemplary Method for Treating Lambert Eaton Myasthenic Syndrome

This example describes an exemplary method for treating Lambert Eaton Myasthenic Syndrome.

Doses should be individualized based on Area Under the Curve (AUC) 0 to 4 hours of blood or plasma amifampridine concentration and use of calculation of the amifampridine dose using the trapezoidal rule or use of a Bayesian Model Informed pharmacokinetic model (noncompartmental PK analysis can use estimates of total drug exposure; and total drug exposure can be estimated by area under the curve (AUC) methods, for example, using the trapezoidal rule (numerical integration)).

### Exemplary Treatment of Lambert Eaton Myasthenic Syndrome (LEMS)

Treatment of adult and pediatric patient 5 years of age and older with Lambert Eaton Myasthenic Syndrome (LEMS)
Starting test dose for the dose escalation phase is 0.1 mg/kg three times daily.
After 1 or more days adjust dose three or four times daily to a target of between about 20 ng/mL*h to 30 ng/mL*h AUC₀₋₄ₕ blood or plasma amifampridine after the PM dose;
After 4 or more days can adjust dose three or four times daily to a target of between about 30 ng/mL*h to 40 ng/mL*h AUC₀₋₄ₕ blood or plasma amifampridine after the PM dose;
After 1 or more weeks days can adjust dose three or four times daily to a target of between about 40 ng/mL*h to 50 ng/mL*h AUC₀₋₄ₕ blood or plasma amifampridine after the PM dose;
After 10 or more days can adjust dose three or four times daily to a target of between about 50 ng/mL*h to 60 ng/mL*h AUC₀₋₄ₕ blood or plasma amifampridine after the PM dose;
After 2 or more weeks can adjust dose three or four times daily to a target of between about 60 ng/mL*h to 75 ng/mL*h AUC₀₋₄ₕ blood or plasma amifampridine after the PM dose;
After 2½ or more weeks can adjust dose three or four times daily to a target of between about 75 ng/mL*h to 90 ng/mL*h AUC₀₋₄ₕ blood or plasma amifampridine after the PM dose;
After 3 or more weeks can adjust dose three or four times daily to a target of between about 90 ng/mL*h to 105 ng/mL*h AUC₀₋₄ₕ blood or plasma amifampridine after the PM dose;
After 3½ or more weeks days can adjust dose three or four times daily to a target of between about 105 ng/mL*h to 120 ng/mL*h AUC₀₋₄ₕ blood or plasma amifampridine after the PM dose;
After 4 or more weeks can adjust dose three or four times daily to a target of between about 120 ng/mL*h to 135 ng/mL*h AUC₀₋₄ₕ blood or plasma amifampridine after the PM dose;
After 4½ or more weeks can adjust dose three or four times daily to a target of between about 135 ng/mL*h to 150 ng/mL*h AUC₀₋₄ₕ blood or plasma amifampridine after the PM dose;
After completing dose escalation phase, readjust the dose monthly to the highest of AUC₀₋₄ₕ blood or plasma amifampridine concentration achieved during the during the dose escalation period;
Amifampridine can cause seizures. Consider discontinuation or dose-reduction of amifampridine in patients who have a seizure while on treatment

### Dose and Blood Sample Collection Timing for Trough Blood Concentration Determination or for AUC Blood Concentration Determination

Calculation of AUC₀₋₄ₕ blood or plasma drug concentration is dependent on the amount and timing of prior drug administration, the timing of blood sample collection, the blood collection device, the laboratory assay method and an individualized and adaptive pharmacokinetic model of drug clearance.

After the test dose, three blood sample collections should occur at about one, two about four hours afterward the first test dose administration.

After, each dose escalation, one to three blood samples are collected and incorporated in the patient-specific model to generate an updated patient specific PK model, a patient-specific next dose to achieve the predicted next target blood or plasma amifampridine concentration.

A single sample is collected at about three hours after the PM administration, two samples are collected immediately prior to the PM dose administration and about three hours afterwards, or three samples are collected immediately prior to PM dose administration and about 2 hours and 4 hours afterwards. This procedure is continued until the patient achieves the predicted optimal target blood or plasma amifampridine concentration.

In the event of dose limiting toxifies, the dose is reduced to the preceding tolerable dose.

There-after, on a monthly basis, at least one sample is collected and incorporated in the patient-specific model to generate an updated patient specific PK model, and a patient-specific dose to achieve the predicted optimal target blood or plasma amifampridine concentration.

Each blood sample should occur immediately after the blood collection device is removed from the blood collection blister card.

The blood samples should be placed in the blood sample pouch.

The pouch should be placed in a shipping box and sent to the designated assay laboratory for the determination of blood amifampridine concentrations.

Drug administration should occur immediately after the amifampridine capsule is removed from the capsule blister card (that has a seven-day supply of capsules).

Any missed dose should not be taken, instead, the patient should use the next specified dose on the blister card.

After seven days, the empty card (or partial empty blister card, in the event of a missed dose) should be placed into a shipping package and sent to the designated assay laboratory for determination of dose timing and adherence.

### Calculation of Amifampridine Dose Using the Linear Trapezoidal Rule:

Adjusted dose (mg)=Actual Dose (mg)×Target AUC0-4h (mg/mL*h)/Actual AUC0-4h (mg/mL*h) using the linear trapezoidal rule.

### Calculation of Blood or Plasma Amifampridine Concentration Calculation Using Bayesian Model Informed Pharmacokinetic Model

In alternative embodiments, a pharmacokinetic (PK) model is used to approximate pharmacokinetic (PK) profiles of patients. For instance, current methods to determine a patient-specific pharmacokinetic profile for amifampridine include performing multiple blood tests. After (test dose, minimum therapeutic dose) of amifampridine is administered, six or more blood draws are performed over 4-hour post-administration period. As can be appreciated, such a procedure is especially taxing on a patient, healthcare provider, and lab because of the numerous separate blood draws.

To address this problem, the exemplary model generator as provided herein is configured to generate relatively accurate pharmacokinetic models based upon a sample of patients with varying body weights, height, renal function, liver function, age, genders, pharmacogenetic polymorphism and concomitant medications. These models are then used to determine or approximate a pharmacokinetic profile of a patient without having to subject a patient to all the blood draws and subsequent analysis.

In alternative embodiments, PK models are determined using patient samples selected from one or more sets of patient data. The patient samples may be, for example, selected among patients who have already been subscribed a therapeutic dosing regimen using the above-described blood draw procedure. The patient samples may also include patients specifically selected to go through the blood draw procedure for the purpose of creating the models. The patient samples may include patients from one hospital or medical system and/or patients associated from multiple hospitals, medical systems, geographic regions, etc.

In alternative embodiments, patient samples include data for patients of varying body weights, height, renal function, liver function, age, genders, pharmacogenetic polymorphism and concomitant drug therapies. In some embodiments, the data for the patients may be separated into body weight and renal function brackets such that a separate model is generated for each bracket. The patient data may additionally or alternatively be partitioned based on pharmacogenetic polymorphism.

In alternative embodiments, post-drug administration (for example, post-amifampridine administration) blood samples are collected from each patient after certain durations of time. In alternative embodiments, the blood samples are collected at different times and/or the number of blood samples collected are fewer or greater. For instance, fewer blood samples may be collected after the initial test dose.

In alternative embodiments, the exemplary model generator creates a PK patient model by performing a Bayesian analysis that uses previous knowledge of blood or plasma amifampridine concentration in the sampled patients over time after administration of the (test dose, minimum therapeutic dose). In some instances, the model generator is configured to analyze each patient's sampled dosing history in conjunction with pre-administration blood or plasma amifampridine concentration levels, so that washout data is not needed to construct the PK models. In other embodiments, the model generator uses patient washout data in conjunction with the post administration blood or plasma amifampridine concentration levels to create one or more pharmacokinetic models. Patient washout data corresponds to a baseline where the patient does not include propranolol in their system.

In alternative embodiments, the exemplary model generator creates the one or more PK models using, for example, the patient sample data. The model generator may combine the individual patient samples into one or more population profiles (for example, weight sets, age sets, renal function, liver function, pharmacogenetic polymorphisms, etc.), which is then used as a basis for the respective pharmacokinetic model. In alternative embodiments, the model generator groups the patient samples for different weight, renal function, ages, and/or pharmacogenetic polymorphisms into different sets. In alternative embodiments, the model generator then performs covariate and statistical modeling on the grouped patient samples of each set to create a population pharmacokinetic model for that set, for example, as described by Thakker et al. (2017) “Population Pharmacokinetics/Pharmacodynamics of 3,4-Diaminopyridine Free Base in Patients With Lambert-Eaton Myasthenia”, CPT Pharmacometrics Syst. Pharmacol. 2017, 6, pg 625-634, the entirety of which is incorporated herein by reference. In alternative embodiments the model generator uses a model and sampled data from other Bayesian analysis techniques (for example, a naïve Bayes classifier).

In this exemplary embodiment, the covariate model used by the model generator determines relationships between pharmacokinetic parameters (for example, how quickly amifampridine is metabolized, etc.) and patient characteristics (for example, body weight, clinical laboratory data, age, etc.). In alternative embodiments, the model generator uses a statistical model to determine variance in pharmacokinetic parameters among the sampled patients in addition to residual variance because of biological variability between patients, measurement errors, and errors within the fit of the sampled data to the pharmacokinetic model.

In alternative embodiments, the exemplary model generator is configured to perform the covariate and statistical modeling using the nonlinear mixed effects modeling software NONMEM version 7.3, is used (ICON Development Solutions, Ellicott City, MD). The first-order conditional estimation method with interaction is implemented for all model runs. Run management is performed using Pirana version 2.9.2. Visual predictive checks (VPCs) and bootstrapping is performed using Perl-speaks-NONMEM version 3.7.6. Data manipulation and visualization is performed using the software R version 3.2.0 (R Foundation for Statistical Computing, Vienna, Austria) and RStudio version 0.99.442 (Rstudio, Boston, MA); with the lattice, xpose4, and ggplot2 packages is used for the latter.

The ability of the final population pharmacokinetic model to describe observed concentration data is evaluated by visual predictive check. A total of 1000 simulations is performed with the final population pharmacokinetic model, and the 90% prediction interval (PI) was calculated and used to overlay the observed data for visual predictive check.

In alternative embodiments, a two-compartment model is used for pharmacokinetic modeling. In the PK analysis, 3,4-DAP and 3-Ac DAP concentrations is modeled simultaneously. One and two-compartment models are tested for both the parent and metabolite. The structural model is selected based on an assessment of the objective function value (OFV), precision of parameter estimates, diagnostic plots, and model stability. Given that 3,4-DAP is largely converted to 3-Ac DAP, the fraction of parent converted to metabolite (Fₘ) is fixed to 1 to obtain an identifiable model. Thus, all clearance and volume parameters for 3-Ac DAP are relative to the Fₘ and the bioavailability of 3,4-DAP (F), which is summarized as F_{3ACDAP} (the product of Fₘ and F). Once the PK model met acceptance criteria, the individual PK estimates is fixed.

Covariates are analyzed for inclusion in the PK components of the model. Visual inspection is performed using box and scatter plots (for categorical and continuous covariates, respectively) of the individual deviations from typical population values in the PK against covariates. In the population PK model, the following covariates are explored: total body weight (TBW), serum creatinine (SCR), age, and gender. A forward inclusion (P<0.05 and DOFV >3.8) and backward elimination (P<0.01 and DOFV >6.6) approach are used to evaluate statistical significance for inclusion of covariates in the model. Descriptive statistics of the individual empirical Bayesian estimates (EBEs) is calculated.

The relationship between TBW and PK parameters specifically is evaluated using an allometric relationship for clearance parameters (systemic clearance (CL/F), intercompartmental clearance (Q/F), and metabolite clearance (CLₘ/F_{3ACDAP})) and a linear scale for volume parameters (V/F for one-compartment model; central compartment volume (V_{c}/F) and peripheral compartment volume for 2-compartment model. Fixed exponents of 0.75 and 1 for clearance and volume parameters, respectively, were applied:

$${Wt}_{i} 0 : 75$$

$$CL = F 5 {CL}_{std}$$

where CL_{std} and V_{std} denote the population estimates of CL/F and V/F, respectively, WTₘ denotes the median TBW of the evaluated patients, and Wtᵢ represents the TBW for the ith patient. Other continuous and categorical covariates were tested using a power model and centered using the median covariate value for the sample.

In alternative embodiments, responsive to creating one or more pharmacokinetic models, the exemplary model generator provides the pharmacokinetic model(s) to the PK server. The transmission may be over a private network, such as a local area network, or over a public network, such as an Internet. The model generator may also store the models to the database, which is also accessible by the PK server via one or more interfaces. In other instances, the model generator may be integrated with the PK server.

In alternative embodiments, the exemplary model generator refines the models for each patient. For instance, the PK server may receive patient specific information including, weight, serum creatinine, and dosing level for previous treatments. The model generator uses the previous treatment information (for example, dosing amounts, intervals, etc.) to refine or adjust the model such that dosing recommendations and a pharmacokinetic profile are more aligned to the specific patient but still account for potential patient variance. The model generator transmits the patient-specific model to the PK server.

In alternative embodiments, the PK server is configured to create patient-specific models using the pharmacokinetic model provided by the model generator to account for the patient-specific pharmacokinetic variance. In this manner, one or more base models are refined or adjusted by the PK server responsive to receiving previous treatment information for a specific patient. The PK server may be configured to store the patient-specific model to the database for subsequent uses by the same healthcare provider or other healthcare providers.

In alternative embodiments, once a PK profile for a patient is generated, the PK server is configured to transmit the PK profile to the drug monitoring tool. In some embodiments, the PK server can encrypt the data file prior to transmission. The encryption can be specific to a particular patient such that the drug monitoring tool can only open and process a received PK profile if the tool has a patient specific authentication key.

In alternative embodiments, methods as provided herein use a drug monitoring tool as described by U.S. Pat. No. 10,896,749, which comprises a data receiver and an interactive user interface, where the data receiver is configured to receive a pharmacokinetic (PK) profile of a patient, and an interactive user interface is configured to display a time-varying therapeutic drug or plasma protein level of the patient. The time-varying therapeutic drug or plasma protein level is based on an administered dose of a drug, and the PK profile of the patient.

In alternative embodiments, the drug monitoring tool used in methods as provided herein comprises: a data receiver configured to receive, from a secured server, a patient pharmacokinetic (PK) profile of a patient, wherein the secured server comprises: (1) a Bayesian model of pharmacokinetic (PK) profiles of sampled patients, the Bayesian model including (i) a blood or plasma drug clearance and (ii) a volume of distribution relationship for a plasma protein or drug based upon at least one of patient body weight and rum creatinine, and (2) a PK server configured to determine the patient PK profile based upon the Bayesian model, and at least one of a bodyweight and serum creatinine of the patient; and an interactive user interface configured to: display to the patient a graphical representation predictive of a time-varying drug level in the patient, the time-varying drug level determined by the drug monitoring tool based on the timing and amount of an administered dose of the drug to the patient and the patient PK profile.

### Dosage Forms and Strengths

In alternative embodiments, bMED amifampridine is available for oral administration in the following dosage forms and strengths and supplied on a blister card that time-stamps the use of the single dose tablet or capsule, or the blood collection device.

In alternative embodiments amifampridine is formulated as tablets, pills, geltabs or capsules for administration provide either 2 mg, 2.5 mg, 3 mg, 3.5 mg, 3 mg, 4 mg, 5 mg, 6 mg, 8 mg, 10 mg, 12 mg, 14 mg, 17 mg, 20 mg, 23 mg, 26 mg, 30 mg, 45 mg, 40 mg, 45 mg, or 50 mg of amifampridine.

### Example 4: Exemplary Method for Treating Lambert Eaton Myasthenic Syndrome with an Accelerated Dose Escalation Phase

This example describes an exemplary method for treating Lambert Eaton Myasthenic Syndrome with an accelerated titration schedule.

Doses should be individualized based on Area Under the Curve (AUC) of blood or plasma amifampridine concentration and use of a Bayesian Model Informed pharmacokinetic model or calculation of the dose using the trapezoidal rule.

Exemplary Treatment of Lambert Eaton Myasthenic Syndrome (LEMS) with an Accelerated Dose Escalation Phase,
Treatment of adult and pediatric patient 5 years of age and older with Lambert Eaton Myasthenic Syndrome (LEMS)
Starting test dose for the dose escalation phase is 0.1 mg/kg three times daily
After 1 or more days adjust dose three or four times daily to a target of between about 30 ng/mL*h to 45 ng/mL*h AUC₀₋₄ₕ blood or plasma amifampridine after the PM dose;
After 4 or more days can adjust dose three or four times daily to a target of between about 45 ng/mL*h to 60 ng/mL*h AUC₀₋₄ₕ blood or plasma amifampridine after the PM dose;
After 1 or more weeks days can adjust dose three or four times daily to a target of between about 60 ng/mL*h to 80 ng/mL*h AUC₀₋₄ₕ blood or plasma amifampridine after the PM dose;
After 10 or more days can adjust dose three or four times daily to a target of between about 80 ng/mL*h to 100 μg/mL*h AUC₀₋₄ₕ blood or plasma amifampridine after the PM dose;
After 2 or more weeks can adjust dose three or four times daily to a target of between about 100 ng/mL*h to 125 ng/mL*h AUC₀₋₄ₕ blood or plasma amifampridine after the PM dose;
After 2 or more weeks can adjust dose three or four times daily to a target of between about 125 μg/mL*h to 150 ng/mL*h AUC₀₋₄ₕ blood or plasma amifampridine after the PM dose;
Readjust dose monthly after the dose escalation phase to the highest of AUC₀₋₄ₕ blood or plasma amifampridine concentration obtained during the dose escalation phase;
Amifampridine can cause seizures. Consider discontinuation or dose-reduction of amifampridine in patients who have a seizure while on treatment.

### Example 5: Exemplary Method for Treating Duchennes Muscular Dystrophy

This example describes exemplary methods for treating Duchennes Muscular Dystrophy, including methods for determining drug dosages for treating Duchennes Muschular Dystrophy.

Doses should be individualized based on Area Under the Curve (AUC) 0-8 h to of blood or plasma 21-desDFZ (the active metabolite of deflazacort) concentration and calculation of the deflazacort dose using the trapezoidal rule or the use of a Bayesian Model Informed pharmacokinetic model.

### Exemplary Treatment of Duchennes Muscular Dystrophy (DMD)

Threatment of children and adults ages 2+. To attain a blood or plasma AUC₀₋₈ₕ target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or 250 ng/mL*h;
Treatment of adult and pediatric patient 2 years of age and older with Duchennes Muscular Dystrophy (DMD)
Starting test dose for the of 0.9 mg/kg daily.
After 1 or more days adjust dose to a target of about 250 ng/mL*h AUC₀₋₈ₕ blood or plasma concentration for 21-desDFZ;
Re-adjust dose until obtain a target of about 250 ng/mL*h AUC₀₋₈ₕ blood or plasma concentration for 21-desDFZ for two consecutive measurements,
Readjust the dose monthly to maintain the target AUC₀₋₈ₕ blood or plasma concentration for 21-desDFZ
   Exemplary Treatment of Duchenne Muscular Dystrophy (DMD) with Dose Titration Phase to a Tolerable Dose,

Treatment of children and adults ages 1+ years. To attain a tolerable blood or plasma AUC₀₋₈ₕ target for 21-desDFZ (the active metabolite of deflazacort) of between about 25 to 1000 ng/mL*h, between about 100 to 600 ng/mL*h, between about 150 to 400 ng/mL*h, between about 200 to 300 ng/mL*h or about 250 ng/mL*h or about 175 ng/mL*h or about 125 ng/mL*h or about 75 ng/mL*h;
Starting test dose of deflazacort for the titration phase is 0.9 mg/kg daily
After 1 or more days adjust dose daily to a target of between about 225 ng/mL*h to 275 ng/mL*h AUC₀₋₈ₕ blood or plasma 21-desDFZ concentration after the AM dose;
After 1 month or more months can adjust dose to a target of between about 150 ng/mL*h to 200 ng/mL*h AUC₀₋₈ₕ blood or plasma 21-desDFZ concentration after the AM dose;
After 2 months or more months can adjust dose daily to a target of between about 100 ng/mL*h to 135 ng/mL*h AUC₀₋₈ₕ blood or plasma 21-desDFZ concentration after the AM dose;
After 3 or more months can adjust dose daily to a target of between about 65 ng/mL*h to 85 μg/mL*h AUC₀₋₈ₕ blood or plasma 21-desDFZ concentation after the AM dose;
Readjust the dose monthly to maintain the tolerable target AUC₀₋₈ₕ blood or plasma concentration for 21-desDFZ;

### Example 6: Exemplary Method for Treating Duchenne Muscular Dystrophy

This example describes an exemplary method for treating Duchenne Muscular Dystrophy with the combination of deflazacort and ataluren.

Doses of deflazacort should be individualized based on Area Under the Curve (AUC) 0-8 h to of blood or plasma 21-desDFZ (the active metabolite of deflazacort) concentration and calculation of the deflazacort dose using the trapezoidal rule or the use of a Bayesian Model Informed pharmacokinetic model. Deflazacort should be titrated to a tolerable blood or plasma concentration of 21-desDFZ. After obtaining a tolerable dose of deflazacort, doses of ataluren should be individualized based on C₆ₕ blood or plasma ataluren concentration after the midday dose or trough blood or plasma ataluren concentration prior to the evening dose or use of a Bayesian Model Informed pharmacokinetic model.

### Exemplary Treatment of Duchenne Muscular Dystrophy (DMD).

After obtaining a tolerable dose of deflazacort, initiate treatment of children and adults at ages 1+ years.
Starting test dose of ataluren is 10 mg/kg body weight in the morning, 10 mg/kg body weight midday and 20 mg/kg body weight in the evening (for total daily dose of 40 mg/kg body weight),
After 1 week adjust the dose, proportionally, three times daily to a target of between about 5 μg/mL to 10 μg/mL C₆ₕ blood or plasma ataluren concentration after the midday dose or trough blood or plasma ataluren concentration prior to the evening dose.
Readjust the dose monthly to maintain the target of between about 5 μg/mL to 10 g/mL C₆ₕ blood or plasma ataluren concentration after the midday dose or trough blood or plasma ataluren concentration prior to the evening dose.

### Dose and Blood Sample Collection Timing for AUC Blood Concentration Determination

Calculation of AUC₀₋₈ₕ blood or plasma 21-desDFZ concentration is dependent on the amount and timing of prior drug administration, the timing of blood sample collection, the blood collection device, the laboratory assay method and an individualized and adaptive pharmacokinetic model of drug clearance.

After the starting dose, three blood sample collections should occur at about one, three about eight hours afterward the starting dose administration. In alternative embodiments, four blood sample collections should occur at about one, two, four and twelve hours afterward the starting dose administration.

Each week or month thereafter, three blood samples are collected and used to calculate the adjusted using the trapezoidal rule the incorporated in the patient-specific model to generate an updated patient specific PK model, a patient-specific next dose to achieve the predicted next target blood or plasma amifampridine concentration.

In the event of dose limiting toxifies, the dose is reduced by 20% until a tolerable dose is achieved.

There-after, on a monthly basis, at least one sample is collected and incorporated in the patient-specific model to generate an updated patient specific PK model, and a patient-specific dose to achieve the predicted optimal target plasma2l-desDFZ concentration.

Each blood sample should occur immediately after the blood collection device is removed from the blood collection blister card.

The blood samples should be placed in the blood sample pouch.

The pouch should be placed in a shipping box and sent to the designated assay laboratory for the determination of blood amifampridine concentrations.

Drug administration should occur immediately after the deflazacort capsule is removed from the capsule blister card (that has a seven-day supply of capsules).

Any missed dose should not be taken, instead, the patient should use the next specified dose on the blister card.

After seven days, the empty card (or partial empty blister card, in the event of a missed dose) should be placed into a shipping package and sent to the designated assay laboratory for determination of dose timing and adherence.

### Calculation of Deflazacort Dose Using the Linear Trapezoidal Rule

In alternative embodiments, the adjusted dose (mg) is calculated using the linear trapezoidal rules as follows:

Adjusted dose (mg) will equal the closest available dose to the Actual Dose (mg)×Target AUC₀₋₄ₕ (mg/mL*h)/Actual AUC₀₋₄ₕ (mg/mL*h) using the linear trapezoidal rule. In alternative embodiments, adjusted dose (mg) will equal the closest available dose to the Actual Dose (mg)×Target AUC₀₋₈ₕ (mg/mL*h)/Actual AUC₀₋₈ₕ (mg/mL*h) using the linear trapezoidal rule.

In alternative embodiments a software tool will calculate the adjusted dose will equal the closest available dose to the Actual Dose (mg)×Target AUC₀₋₄ₕ (mg/mL*h)/Actual AUC₀₋₄ₕ (mg/mL*h) using the linear trapezoidal rule. In alternative embodiments, that adjusted dose (mg)=closest available dose to the Actual Dose (mg)×Target AUC₀₋₈ₕ (mg/mL*h)/average of the Actual AUC₀₋₈ₕ (mg/mL*h) using the linear trapezoidal rule for the current and two previous months.

### Calculation of Deflazacort Dose Using the Logarithmic Trapezoidal Rule

In alternative embodiments, the adjusted dose (mg) is calculated using the logarithmic trapezoidal rules as follows:

Adjusted dose (mg) will equal the closest available dose to the Actual Dose (mg)×Target AUC₀₋₈ₕ (mg/mL*h)/Actual AUC₀₋₈ₕ (mg/mL*h) using the logarithmic trapezoidal rule. In alternative embodiments, adjusted dose (mg) will equal the closest available dose to the Actual Dose (mg)×Target AUC₀₋₁₂ₕ (mg/mL*h)/Actual AUC₀₋₁₂ₕ (mg/mL*h) using the logarithmic trapezoidal rule.

In alternative embodiments a software tool will calculate the adjusted dose will equal the closest available dose to the Actual Dose (mg)×Target AUC₀₋₈ₕ (mg/mL*h)/Actual AUC₀₋₈ₕ (mg/mL*h) using the logarithmic trapezoidal rule. In alternative embodiments, that adjusted dose (mg)=closest available dose to the Actual Dose (mg)×Target AUC₀₋₁₂ₕ (mg/mL*h)/average of the Actual AUC₀₋₁₂ₕ (mg/mL*h) using the logarithmic trapezoidal rule for the current and two previous months.

Calculation of Blood or Plasma 21-desDFZ Calculation Using Bayesian Model Informed Pharmacokinetic Model

In alternative embodiments leverage a pharmacokinetic (PK) model to closely mirror PK profiles of individual patients in a community setting. The illustrative PK model generator delineated herein is capable of devising considerably precise pharmacokinetic models founded on a sample set of patients with diverse body weights, heights, renal and liver functions, ages, genders, pharmacogenetic variations, and concurrent medication regimens.

Various embodiments utilize patient samples derived from one or more patient data sets collected in a community setting, and optionally in medical office, hospital and specialized testing centers. The selected samples are collected from patients prescribed bMED™ deflazacort and utilizing the bMED™ kit, inclusive of time-stamped individual dosage amounts, time-stamped blood sample collection apparatus, and sampling methodologies, and optionally, from patient samples collected from patients specifically selected to partake in the blood extraction process to aid in model creation and utilizing the bMED™ kit, inclusive of time-stamped individual dosage amounts, time-stamped blood sample collection apparatus, and sampling methodologies, and optionally, utilizing venous blood sampling.

In differing embodiments, patient samples encompass data for patients with assorted body weights, heights, renal and liver functions, ages, genders, pharmacogenetic variations, and concomitant medication regimens. In some cases, patient data can be divided into brackets based on body weight and renal function, allowing for the creation of individual models for each bracket. The data could be partitioned based on pharmacogenetic variations as an alternative or additional measure.

Certain embodiments entail the collection of blood samples post-deflazacort administration from each patient after specified time periods. The collected samples may vary in terms of the time of collection and/or the total number of collected samples. For example, additional samples might be collected post the initial test dosage.

In certain embodiments, the model generator establishes a PK patient model through a Bayesian analysis, incorporating prior knowledge of blood or plasma 21-desDFZ concentrations over time in the sampled patients following the administration of a test dose or minimum therapeutic dose. In some cases, the model generator is designed to examine each patient's recorded dosing history in conjunction with pre-administration blood or plasma 21-desDFZ concentration levels, thereby eliminating the need for washout data to formulate the PK models. Conversely, in other embodiments, the model generator employs patient washout data alongside post administration blood or plasma 21-desDFZ concentration levels to establish one or more pharmacokinetic models.

The illustrative model generator can create one or more PK models using patient sample data. It may amalgamate individual patient samples into one or more population profiles, which then serve as the basis for the respective pharmacokinetic model. It may also segregate the patient samples based on different concomitant medications, weights, renal functions, ages, and/or pharmacogenetic variations into discrete sets. The model generator can then conduct covariate and statistical modeling on the grouped patient samples of each set to create a population pharmacokinetic model for that set.

In the provided exemplary embodiment, the covariate model applied by the model generator establishes relationships between pharmacokinetic parameters and patient characteristics. The model generator might use a statistical model to determine variance in pharmacokinetic parameters among the sampled patients in addition to residual variance due to biological variability between patients, measurement inaccuracies, and errors within the fit of the sampled data to the pharmacokinetic model.

In various embodiments, the illustrative model generator is designed to execute covariate and statistical modeling using the nonlinear mixed effects modeling software NONMEM version 7.3. The first-order conditional estimation method with interaction is implemented for all model runs. Run management is facilitated using Pirana version 2.9.2. Visual predictive checks and bootstrapping is performed using Perl-speaks-NONMEM version 3.7.6. Data manipulation and visualization is executed using the software R version 3.2.0 and RStudio version 0.99.442, with the lattice, xpose4, and ggplot2 packages used for the latter.

In alternative embodiments, a two-compartment model is employed for pharmacokinetic modeling. In the PK analysis, 21-desDFZ concentrations are modeled simultaneously. One, or two-compartment models are tested for both the parent and metabolite. The structural model is selected based on an evaluation of the objective function value, precision of parameter estimates, diagnostic plots, and model stability.

In various embodiments, after devising one or more pharmacokinetic models, the illustrative model generator delivers the pharmacokinetic model(s) to the PK server. The transmission can occur over a private or public network. Alternatively, the model generator may store the models in a database accessible by the PK server via one or more interfaces.

In several embodiments, the model generator refines the models for each patient using previously recorded treatment information such as weight, serum creatinine, and dosing level and interval. In this way, the PK server can create patient-specific models accounting for the individual pharmacokinetic variance. Once a PK profile for a patient is generated, it can be transmitted to the drug monitoring tool.

Alternative embodiments implement a drug monitoring tool comprising a data receiver and an interactive user interface, configured to display a time-varying 21-desDFZ level of the patient, founded on an administered drug dose and the patient's PK profile.

In alternative embodiments, a pharmacokinetic (PK) model is used to approximate pharmacokinetic (PK) profiles of patients. For instance, current methods to determine a patient-specific pharmacokinetic profile for deflazacort include performing multiple blood tests. After (test dose, minimum therapeutic dose) of amifampridine is administered, six or more blood draws are performed over 4-hour post-administration period. As can be appreciated, such a procedure is especially taxing on a patient, healthcare provider, and lab because of the numerous separate blood draws.

To address this problem, the exemplary model generator as provided herein is configured to generate relatively accurate pharmacokinetic models based upon a sample of patients with varying body weights, height, renal function, liver function, age, genders, pharmacogenetic polymorphism and concomitant medications. These models are then used to determine or approximate a pharmacokinetic profile of a patient without having to subject a patient to all the blood draws and subsequent analysis.

In alternative embodiments, PK models are determined using patient samples selected from one or more sets of patient data. The patient samples may be, for example, selected among patients who have already been subscribed a therapeutic dosing regimen using the above-described blood draw procedure. The patient samples may also include patients specifically selected to go through the blood draw procedure for the purpose of creating the models. The patient samples may include patients from one hospital or medical system and/or patients associated from multiple hospitals, medical systems, geographic regions, etc.

In alternative embodiments, patient samples include data for patients of varying body weights, height, renal function, liver function, age, genders, pharmacogenetic polymorphism and concomitant drug therapies. In some embodiments, the data for the patients may be separated into body weight and renal function brackets such that a separate model is generated for each bracket. The patient data may additionally or alternatively be partitioned based on pharmacogenetic polymorphism.

In alternative embodiments, post-drug administration (for example, post-deflazacort administration) blood samples are collected from each patient after certain durations of time. In alternative embodiments, the blood samples are collected at different times and/or the number of blood samples collected are fewer or greater. For instance, fewer blood samples may be collected after the initial test dose.

In alternative embodiments, the exemplary model generator creates a PK patient model by performing a Bayesian analysis that uses previous knowledge of blood or plasma 21-desDFZ concentration in the sampled patients over time after administration of the (test dose, minimum therapeutic dose). In some instances, the model generator is configured to analyze each patient's sampled dosing history in conjunction with pre-administration blood or plasma 21-desDFZ concentration levels, so that washout data is not needed to construct the PK models. In other embodiments, the model generator uses patient washout data in conjunction with the post administration blood or plasma amifampridine concentration levels to create one or more pharmacokinetic models. Patient washout data corresponds to a baseline where the patient does not include 21-desDFZ in their system.

In alternative embodiments, the exemplary model generator creates the one or more PK models using, for example, the patient sample data. The model generator may combine the individual patient samples into one or more population profiles (for example, weight sets, age sets, renal function, liver function, pharmacogenetic polymorphisms, etc.), which is then used as a basis for the respective pharmacokinetic model. In alternative embodiments, the model generator groups the patient samples for different weight, renal function, ages, and/or pharmacogenetic polymorphisms into different sets. In alternative embodiments, the model generator then performs covariate and statistical modeling on the grouped patient samples of each set to create a population pharmacokinetic model for that set, for example, as described by Thakker et al. (2017) “Population Pharmacokinetics/Pharmacodynamics of 3,4-Diaminopyridine Free Base in Patients With Lambert-Eaton Myasthenia”, CPT Pharmacometrics Syst. Pharmacol. 2017, 6, pg 625-634, the entirety of which is incorporated herein by reference. In alternative embodiments the model generator uses a model and sampled data from other Bayesian analysis techniques (for example, a naïve Bayes classifier).

In this exemplary embodiment, the covariate model used by the model generator determines relationships between pharmacokinetic parameters (for example, how quickly 21-desDFZ is metabolized, etc.) and patient characteristics (for example, body weight, clinical laboratory data, age, etc.). In alternative embodiments, the model generator uses a statistical model to determine variance in pharmacokinetic parameters among the sampled patients in addition to residual variance because of biological variability between patients, measurement errors, and errors within the fit of the sampled data to the pharmacokinetic model.

In alternative embodiments, the exemplary model generator is configured to perform the covariate and statistical modeling using the nonlinear mixed effects modeling software NONMEM version 7.3, is used (ICON Development Solutions, Ellicott City, MD). The first-order conditional estimation method with interaction is implemented for all model runs. Run management is performed using Pirana version 2.9.2. Visual predictive checks (VPCs) and bootstrapping is performed using Perl-speaks-NONMEM version 3.7.6. Data manipulation and visualization is performed using the software R version 3.2.0 (R Foundation for Statistical Computing, Vienna, Austria) and RStudio version 0.99.442 (RStudio, Boston, MA); with the lattice, xpose4, and ggplot2 packages is used for the latter.

The ability of the final population pharmacokinetic model to describe observed concentration data is evaluated by visual predictive check. A total of 1000 simulations is performed with the final population pharmacokinetic model, and the 90% prediction interval (PI) was calculated and used to overlay the observed data for visual predictive check.

In alternative embodiments, a two-compartment model is used for pharmacokinetic modeling. In the PK analysis, 21-desDFZ concentrations is modeled simultaneously. One and two-compartment models are tested for both the parent and metabolite. The structural model is selected based on an assessment of the objective function value (OFV), precision of parameter estimates, diagnostic plots, and model stability. Once the PK model met acceptance criteria, the individual PK estimates is fixed.

Covariates are analyzed for inclusion in the PK components of the model. Visual inspection is performed using box and scatter plots (for categorical and continuous covariates, respectively) of the individual deviations from typical population values in the PK against covariates. In the population PK model, the following covariates are explored: total body weight (TBW), serum creatinine (SCR), age, and gender. A forward inclusion (P<0.05 and DOFV >3.8) and backward elimination (P<0.01 and DOFV >6.6) approach are used to evaluate statistical significance for inclusion of covariates in the model. Descriptive statistics of the individual empirical Bayesian estimates (EBEs) is calculated.

The relationship between TBW and PK parameters specifically is evaluated using an allometric relationship for clearance parameters (systemic clearance (CL/F), intercompartmental clearance (Q/F), and metabolite clearance (CLₘ/F_{3ACDAP})) and a linear scale for volume parameters (V/F for one-compartment model; central compartment volume (V_{c}/F) and peripheral compartment volume for 2-compartment model. Fixed exponents of 0.75 and 1 for clearance and volume parameters, respectively, were applied:

$${Wt}_{i} 0 : 75$$

$$CL = F 5 {CL}_{std}$$

where CL_{std} and V_{std} denote the population estimates of CL/F and V/F, respectively, WTₘ denotes the median TBW of the evaluated patients, and WTᵢ represents the TBW for the ith patient. Other continuous and categorical covariates were tested using a power model and centered using the median covariate value for the sample.

In alternative embodiments, responsive to creating one or more pharmacokinetic models, the exemplary model generator provides the pharmacokinetic model(s) to the PK server. The transmission may be over a private network, such as a local area network, or over a public network, such as an Internet. The model generator may also store the models to the database, which is also accessible by the PK server via one or more interfaces. In other instances, the model generator may be integrated with the PK server.

In alternative embodiments, the exemplary model generator refines the models for each patient. For instance, the PK server may receive patient specific information including, weight, serum creatinine, and dosing level for previous treatments. The model generator uses the previous treatment information (for example, dosing amounts, intervals, etc.) to refine or adjust the model such that dosing recommendations and a pharmacokinetic profile are more aligned to the specific patient but still account for potential patient variance. The model generator transmits the patient-specific model to the PK server.

In alternative embodiments, the PK server is configured to create patient-specific models using the pharmacokinetic model provided by the model generator to account for the patient-specific pharmacokinetic variance. In this manner, one or more base models are refined or adjusted by the PK server responsive to receiving previous treatment information for a specific patient. The PK server may be configured to store the patient-specific model to the database for subsequent uses by the same healthcare provider or other healthcare providers.

In alternative embodiments, once a PK profile for a patient is generated, the PK server is configured to transmit the PK profile to the drug monitoring tool. In some embodiments, the PK server can encrypt the data file prior to transmission. The encryption can be specific to a particular patient such that the drug monitoring tool can only open and process a received PK profile if the tool has a patient specific authentication key.

In alternative embodiments, methods as provided herein use a drug monitoring tool as described by U.S. Pat. No. 10,896,749, which comprises a data receiver and an interactive user interface, where the data receiver is configured to receive a pharmacokinetic (PK) profile of a patient, and an interactive user interface is configured to display a time-varying 21-desDFZ level of the patient. The time-varying therapeutic 21-desDFZ level is based on an administered dose of a drug, and the PK profile of the patient.

In alternative embodiments, the drug monitoring tool used in methods as provided herein comprises: a data receiver configured to receive, from a secured server, a patient pharmacokinetic (PK) profile of a patient, wherein the secured server comprises: (1) a Bayesian model of pharmacokinetic (PK) profiles of sampled patients, the Bayesian model including (i) a blood or plasma 21-desDFZ clearance and (ii) a volume of distribution relationship for a blood or plasma drug based upon at least one of patient body weight and rum creatinine, and (2) a PK server configured to determine the patient PK profile based upon the Bayesian model, and at least one of a bodyweight and serum creatinine of the patient; and an interactive user interface configured to: display to the patient a graphical representation predictive of a time-varying drug level in the patient, the time-varying drug level determined by the drug monitoring tool based on the timing and amount of an administered dose of the drug to the patient and the patient PK profile.

### Dosage Forms and Strengths

In alternative embodiments, bMED deflazacort is available for oral administration in the following dosage forms and strengths and supplied on a blister card that time-stamps the use of the single dose tablet or capsule, or the blood collection device.

In alternative embodiments, deflazacort is formulated as tablets, pills, geltabs or capsules for administration provide either 6 mg, 7 mg, 8 mg, 9 mg, 10 mg, 12 mg, 14 mg, 16 mg, 18 mg, 20 mg, 23 mg, 26 mg, 30 mg, 34 mg, 38 mg, 43 mg, 48 mg, 54 mg, 60 mg, 66 mg, 72 mg or 80 mg of deflazacort.

### Dosage Forms and Strengths

In alternative embodiments, bMED ataluren is available for oral administration in the following dosage forms and strengths and supplied on a blister card that timestamps the use of the single dose sachet or capsule, or the blood collection device.

In alternative embodiments, ataluren is formulated as a sachet or as pills, tablets, geltabs or capsules for administration provide of ataluren at for example 50 mg, 60 mg, 70 mg, 85 mg, 105 mg, 125 mg, 150 mg, 180 mg, 210 mg, 250 mg, 260 mg, 310 mg, 375 mg, 450 mg, 525 mg, 650 mg, 775 mg, 925 mg, 1000 mg, 1100 mg, 1350 mg, 1600 mg, 1900 mg or 2300 mg of ataluren granules for oral suspension.

### Example 7: Method for Treating Tuberous Sclerosis Complex (TSC)-Associated Partial Onset Seizures

This example describes methods for treating TSC-associated partial onset seizures, including methods for determining drug dosages for treating TSC-associated partial onset seizures.

Doses should be individualized based on achieving a steady state trough blood drug concentration of everolimus and calculation of the everolimus dose using pharmacokinetic formula or the use of a Bayesian Model Informed pharmacokinetic model.

Treatment of children and adults ages two years of age and older with TSC-associated partial-onset seizers. To attain a blood trough target for everolimus of between about 5 to 15 ng/mL or 10 ng/mL:
Starting dose of 5 mg/m² orally once daily,
After 1 or 2 weeks adjust dose to a target of about 10 ng/mL trough blood concentration of everolimus,
Re-adjust dose every 1 or 2 weeks until obtain a target of about 10 ng/mL trough blood concentration for everolimus for two consecutive periods,
Re-adjust the dose monthly to maintain the target trough blood concentration for everolimus.

### Exemplary Treatment of TSC-Associated Partial-Onset Seizures

Treatment of children and adults ages two years of age and older with TSC-associated partial-onset seizers. To attain a blood AUC₀₋₂₄ target for everolimus of between about 50 to 1,000 ng/mL*h or 500 ng/mL*h;
Starting dose for the of 5 mg/m² orally once daily.
After 1 or 2 weeks adjust dose to a target of about 500 ng/mL*h AUC₀₋₂₄ blood concentration of everolimus,
Re-adjust dose every 1 or 2 until obtain a target of about 500 ng/mL*h AUC₀₋₂₄ blood concentration for everolimus for two consecutive periods,
Readjust the dose monthly to maintain the target AUC₀₋₂₄ blood concentration for everolimus.
   Treatment of TSC-Associated Partial-Onset Seizures with Dose Titration Phase to a Tolerable Dose
   Treatment of children and adults ages two years of age and older with TSC-associated partial-onset seizers. To titrate everolimus to a trough blood drug concentration target of 12 ng/mL or a tolerable trough concentration:
Starting dose of 5.0 mg/m² orally once daily,
After 1 or 2 weeks adjust the dose to a target of 5 ng/mL to 8 ng/trough blood concentration,
After 1 or 2 weeks adjust the dose to a target of about 8 ng/mL to 12 ng/mL trough blood concentration,
After 1 or 2 weeks adjust the dose to a target of about 10 ng/mL to 15 ng/mL trough blood concentration,
Re-adjust dose every 1 or 2 weeks until a target of about 12 ng/mL trough blood concentration for everolimus for two consecutive periods is obtained,
In the event of intolerable side effects, reduce dose to the tolerable trough blood concentration,
Re-adjust the dose monthly to maintain the target 12 ng/mL trough blood concentration or the tolerable trough blood concentration for everolimus.
   Treatment of TSC-Associated Partial-Onset Seizures with Selection of a Safe Starting Dose
   Treatment of children and adults ages two years of age and older with TSC-associated partial-onset seizers. To select a safe starting dose of everolimus and titrate to a trough blood drug concentration target of 12 ng/mL or a tolerable trough concentration:
Administer a Test Dose of 5.0 mg/m², draw blood samples, and calculate the Test Dose steady-state,
Starting dose equal to the target of about 5 ng/mL to 7 ng/mL*Test Dose amount/Test Dose steady-state,
After 1 or 2 weeks adjust the dose to about 8 ng/mL to 12 ng/mL trough*average of the actual dose/actual blood drug concentration and test dose/Test Dose steady-state,
After 1 or 2 weeks adjust the dose to about 10 ng/mL to 15 ng/mL trough*moving average of the actual doses/actual blood drug concentrations and test dose/Test Dose steady-state,
Re-adjust dose every 1 or 2 weeks until a moving average blood drug concentration of about 12 ng/mL trough blood concentration for everolimus at least two consecutive periods is obtained,
In the event of intolerable side effects, reduce dose to the tolerable trough blood concentration,
Re-adjust the dose monthly or quarterly to maintain the moving average of blood drug concentration of about 12 ng/mL trough blood concentration for everolimus.
   Treatment of TSC-Associated Partial-Onset Seizures with Safe and Accelerated Titration to an Effective and Tolerable Blood Drug Concentration
   Treatment of children and adults ages two years of age and older with TSC-associated partial-onset seizers. To select a safe starting dose of everolimus and titrate to a trough blood drug concentration target of 12 ng/mL or a tolerable trough concentration:
Administer a Test Dose of 5.0 mg/m², draw blood samples, and calculate Test Dose steady-state,
Starting dose equal to the target of 5 ng/mL to 7 ng/mL*Test Dose amount/Test Dose steady-state, and, optionally, a loading dose,
After 1 or 2 weeks adjust the dose to about 8 ng/mL to 12 ng/mL trough*average of the actual dose/actual blood drug concentration and test dose/Test Dose steady-state, and, optionally, a loading dose,
After 1 or 2 weeks adjust the dose to about 10 ng/mL to 15 ng/mL trough*moving average of the actual doses/actual blood drug concentrations and test dose/Test Dose steady-state, and optionally, a loading dose,
Re-adjust dose every 1 or 2 weeks to obtain a moving average blood drug concentration of about 12 ng/mL trough blood concentration for at least two consecutive periods,
In the event of intolerable side effects, reduce dose to the tolerable trough blood concentration,
Re-adjust the dose monthly or quarterly to maintain the moving average of blood drug concentration of about 12 ng/mL trough blood concentration for everolimus.

### Dosage Forms and Strengths

In alternative embodiments, bMED everolimus is available for oral administration in the following dosage forms and strengths and supplied on a blister card that time-stamps the use of the single dose tablet, capsule, or pre-filled syringe of everolimus solution or the blood collection device.

In alternative embodiments, everolimus is formulated as tablets, pills, geltabs or capsules or pre-filled syringe for administration provide 0.5 mg, 0.6 mg, 0.7 mg, 0.85 mg, 1.05 mg, 1.25 mg, 1.5 mg, 1.8 mg, 2.1 mg, 2.5 mg, 3.0 mg, 3.6 mg, 4.3 mg, 5.0 mg, 6.0 mg, 7 mg, 8.5 mg, 10 mg, 12 mg, 15 mg, 18 mg, 21 mg, 25 mg, 30 mg, 35 mg or 40 mg of everolimus.

### Example 8: Method for Treating Tuberous Sclerosis Complex (TSC)-Renal Angiomyolipomas

This example describes methods for treating TSC-associated renal angiomyolipomas, including methods for determining drug dosages for treating TSC-associated renal angiomyolipomas.

Doses should be individualized based on achieving a steady state trough blood drug concentration of everolimus and calculation of the everolimus dose using pharmacokinetic formula or the use of a Bayesian Model Informed pharmacokinetic model.

Treatment of Adults with renal angiomyolipoma and tuberous sclerosis complex (TSC), not requiring immediate surgery, to attain a trough blood drug concentration of target of about 8 ng/mL or a tolerable dose.
Starting dose of 10 mg orally once daily,
After 1 or 2 weeks adjust dose to a target of about 8 ng/mL trough blood concentration of everolimus,
Re-adjust dose every 1 or 2 weeks until obtain a target of about 8 ng/mL trough blood concentration 2 to 4 week moving average for everolimus for two consecutive periods,
Re-adjust the dose monthly to maintain the quarterly moving trough blood concentration of 8 ng/mL for everolimus.

### Example 9: Method for Treating Breast Cancer

This example describes methods for treating hormone receptor-positive, HER2-negative breast cancer, including methods for determining drug dosages for treating hormone receptor-positive, HER2-negative breast cancer.

Doses should be individualized based on achieving a steady state trough blood drug concentration of everolimus and calculation of the everolimus dose using pharmacokinetic formula or the use of a Bayesian Model Informed pharmacokinetic model.

Treatment of Postmenopausal women with advanced hormone receptor-positive, HER2-negative breast cancer in combination with exemestane after failure of treatment with letrozole or anastrozole. To attain a blood trough target for everolimus of between about 12 to 18 ng/mL or 15 ng/mL or a tolerable trough blood drug concentration.
Administer a Test Dose of 5.0 mg/m², draw blood samples, and calculate Test Dose steady-state,
Starting dose equal to the target of 5 ng/mL*Test Dose amount/Test Dose steady-state, and, optionally, a loading dose,
After 2 or 3 weeks adjust the dose to about 8 ng/mL to 12 ng/mL trough*average of the actual dose/actual blood drug concentration and test dose/Test Dose steady-state, and, optionally, a loading dose,
After 2 or 3 weeks adjust the dose to about 12 ng/mL to 18 ng/mL trough*6 to 9 week moving average of the actual doses/actual blood drug concentrations and test dose/Test Dose steady-state, and optionally,
Re-adjust dose every 2 or 4 weeks to obtain a 8 to 12 week moving average blood drug concentration of about 15 ng/mL trough blood concentration for at least two consecutive periods,
In the event of intolerable side effects, reduce dose to the tolerable trough blood concentration,
Re-adjust the dose monthly or quarterly to maintain the quarterly moving average of trough blood drug concentration of about 15 ng/mL for everolimus.

### Example 10: Methods for Treating Juvenile Polyposis

This example describes methods for treating Juvenile Polyposis, including methods for determining drug dosages for treating Juvenile Polyposis.

Doses should be individualized based on achieving a steady state trough blood drug concentration of everolimus and calculation of the everolimus dose using pharmacokinetic formula or the use of a Bayesian model informed pharmacokinetic model.

Treatment of infant and children ages 6 months and older with Juvenile Polyposis. To attain a blood trough target for everolimus of between about 3 to 7 ng/mL or 5 ng/mL or a tolerable trough blood concentration:
Administer a Test Dose of 5.0 mg/m², draw blood samples, and calculate the Test Dose steady-state,
Starting BID dose equal to the target of about 3 ng/mL to 7 ng/mL*Test Dose amount/Test Dose steady-state,
After 1 or 2 weeks adjust the BID dose to about 5 ng/mL trough*average of the actual dose/actual blood drug concentration and test dose/Test Dose steady-state,
Re-adjust dose every 1 or 2 weeks until a moving average blood drug concentration of about 5 ng/mL trough blood concentration for everolimus at least two consecutive periods is obtained,
In the event of intolerable side effects, reduce dose to the tolerable trough blood concentration,
Re-adjust the dose monthly or quarterly to maintain the quarterly moving average of the trough blood drug concentration of about 5 ng/mL for everolimus.

### Example 11: Method for Treating Pulmonary Arterial Hypertension (PAH)

This example describes methods for treating Pulmonary Arterial Hypertension (PAH), including methods for determining drug dosages for treating Pulmonary Arterial Hypertension (PAH).

Doses should be individualized based on achieving a steady state trough blood drug concentration of everolimus and calculation of the everolimus dose using pharmacokinetic formula or the use of a Bayesian Model Informed pharmacokinetic model.

Treatment of adults with Pulmonary Arterial Hypertension. To attain a blood trough target for everolimus of between about 5 to 8 ng/mL or 6 ng/mL or a tolerable trough blood drug concentration:
Starting dose of 2.5 mg/m² orally twice daily (BID),
After 1 or 2 weeks adjust BID dose to a target of about 6 ng/mL trough blood concentration of everolimus,
Re-adjust BID dose every 1 or 2 weeks until obtain a target of about 6 ng/mL trough blood concentration for everolimus for two consecutive periods,
In the event of intolerable side effects, reduce dose to the tolerable trough blood concentration,
Re-adjust the dose monthly or quarterly to maintain the moving average of blood drug concentration of about 6 ng/mL trough blood concentration for everolimus.

A number of embodiments of the invention have been described. Nevertheless, it can be understood that various modifications may be made without departing from the spirit and scope of the invention. Accordingly, other embodiments are within the scope of the following claims.

1: A therapeutic drug and a companion diagnostic combination, wherein the companion diagnostic comprises a dried blood spot (DBS), a volumetric dried blood spot or a volumetric absorptive microsampling (VAMS), collection device, or an at-home high-volume liquid blood collection device optionally with a time stamp,
wherein the at least one drug, drug combination or formulation is in a group selected from (or is selected from the group consisting of):
(a) a beta adrenergic blocker (or beta blocker), or a salt, hydrate, solvate, tautomer, stereoisomer or deuterated isoform thereof, wherein optionally the beta adrenergic blocker comprises propranolol, atenolol, metoprolol, nadolol, timolol or a combination thereof;
(b) a glucocorticoid, or a salt, hydrate, solvate, tautomer, stereoisomer or deuterated isoform thereof, wherein optionally the glucocorticoid comprises deflazacort, vamorolone (or 17a,21-Dihydroxy-16a-methylpregna-I,4,9(II)-triene-3, 20-dione), prednisone, prednisolone or a combination thereof.
(c) an mTOR (mammalian target of rapamycin) inhibitor, or a salt, hydrate, solvate, tautomer, stereoisomer or deuterated isoform thereof, wherein optionally the mTOR inhibitor comprises everolimus, sirolimus (or rapamycin) or a combination thereof,
(d) a phosphatidylinositol-4,5-bisphosphate 3-kinase, catalytic subunit alpha inhibitor (or PIK3Ca inhibitor, or pl 10a protein), or a salt, hydrate, solvate, tautomer, stereoisomer or deuterated isoform thereof, wherein optionally the PIK3Ca inhibitor comprise alpelisib, inavolisib (or GDC-0077 (Genentech)), serabelisib (or [6-(2-amino-1,3-benzoxazol-5-yl)imidazo[1,2-a]pyridin-3-yl]-morpholin-4-ylmethanone), CYH-33, MN1611 or a combination thereof;
(e) a voltage-gated potassium channel blocker, or a salt, hydrate, solvate, tautomer, stereoisomer or deuterated isoform thereof, wherein optionally the voltagegated potassium channel blocker comprises amifampridine, dalfampridine (or 4-aminopyridine, or pyridin-4-amine), and/or
(f) a selective KCNQ (Kv7) potassium channel opener or salt or a salt, hydrate, solvate, tautomer, stereoisomer or deuterated isoform thereof, wherein optionally the selective potassium channel opener comprises Flurpirtine, Retigabine.

2: The therapeutic drug and companion diagnostic of claim 1, wherein the therapeutic drug further comprises at least a second drug, or comprises at least two drugs comprising:
(a) a beta adrenergic blocker and an mTOR inhibitor;
(b) a glucocorticoid and an mTOR inhibitor;
(c) a glucocorticoid and a voltage-gated potassium channel blocker;
(d) an mTOR inhibitor and a PIK3Ca inhibitor; or
(e) any combination of two or more drugs of claim 1(a) to (e).

3: The drug and companion diagnostic, or therapeutic drug combination and companion diagnostic, of claim 1, further comprising:
(a) casimersen;
(b) viltolarsen,
(c) ataluren,
(d) PF-06939926™ (Pfizer),
(e) SRP-9001™ (Sarepta Therapeutics, Roche),
(f) SPR-5051™ (Sarepta Therapeutics),
(g) DS-5141b™ (Daiichi-Sankyo); or
(h) any combination of (a) to (g).

4: The drug and companion diagnostic, or therapeutic drug combination and companion diagnostic, of claim 1, wherein two or more of the drugs are formulated as separate compositions, or two or more of the drugs are formulated into one composition or drug formulation (two or more drugs are formulated together).

5: The drug and companion diagnostic, or therapeutic drug combination and companion diagnostic, of claim 1, wherein one or two or more of the drugs are packaged individually, or are packaged together, or packaged in any combination, in a single package, a plurality of packages or packettes, or a blister packet, lidded blister or blister card or packets, or a shrink wrap.

6: The drug and companion diagnostic, or therapeutic drug combination and companion diagnostic, of claim 1, wherein one or two or more or all of the drugs are formulated or manufactured as a parenteral formulation, an aqueous solution, a liposome, an injectable solution, a tablet, a pill, a lozenge, a capsule, a caplet, a spray, a sachet, an inhalant, a powder, a freeze-dried powder, an inhalant, a patch, a gel, a geltab, a nanosuspension, a nanoparticle, a nanoliposome, a microgel, a pellet, a suppository or any combination thereof,
and optionally the drug delivery device or product of manufacture is or comprises an implant.

7: The drug and companion diagnostic, or therapeutic drug combination and companion diagnostic, of claim 1, wherein the one or two or more or all of the drugs are formulated or manufactured together in one parenteral formulation, one aqueous solution, one liposome, one injectable solution, one freeze-dried powder, one feed, one food, one food supplement, one pellet, one lozenge, one liquid, one elixir, one aerosol, one inhalant, one adhesive, one spray, one powder, one freeze-dried powder, one patch, one tablet, one pill, one capsule, one gel, one geltab, one lozenge, one caplet, one sachet, one nanosuspension, one nanoparticle, one nanoliposome, one microgel or one suppository.

8: The drug and companion diagnostic, or therapeutic drug combination and companion diagnostic, of claim 1, wherein the one or two or more or all of the drugs are packaged in dosages that match a chrono-dosing regimen to match an optimal dose for the time of day.

9: The drug and companion diagnostic, or therapeutic drug combination and companion diagnostic, of claim 1, wherein the one or two or more or all of the drugs are formulated in a unit dosage amount ranging from between 0.1 mg to about 1 gram, and optionally the one or two or more or all of the drugs are formulated as immediate release formulations or controlled release formulations.

10: A method for treating, ameliorating, preventing, slowing the advance of, or lessoning the symptoms of:
a vascular anomaly,
and optionally the vascular anomaly comprises infantile hemangioma, Arteriovenous Malformation (AVM), Cerebral Cavernous Malformation (CCM), Hereditary Hemorrhagic Telangiectasia (HHT), Von Hippel-Lindau Disease (VHL), angiosarcoma, Kaposiform Hemangioendothelioma (KHE), Kaposiform Lymphangioendothelioma (KLA). Microcystic Lymphatic Malformations or a PIKCA-Related Overgrowth Spectrum (PROS), and optionally the PROS is: Fibro-adipose vascular anomaly Hemihyperplasia—multiple lipomatosis syndrome, CLOVES (congenital lipomatous (fatty) overgrowth, vascular malformations, epidermal nevi and scoliosis/skeletal/spinal anomalies) syndrome Macrodactyly, Facial infiltrating lipomatosis, Macrocephaly-capillary malformation, Dysplastic megalencephaly or Klippel-Trenaunay syndrome;
a Muscular Dystrophy (MD),
and optionally the MD is Duchenne Muscular Dystrophy (DMD), Beckers Muscular Dystrophy, or Lambert Eaton Myasthenic Syndrome (LEMS);
a nervous system disorder,
and optionally the nervous system disorder is Tuberous Sclerosis Complex (TSC), or subependymal giant cell astrocytoma (SEGA) associated with TSC, or a developmental and Epileptic Encephalopathies, a Dravet Syndrome, a Epilepsy in Infancy with Migrating Focal Seizures, a Landau Kleffner Syndrome, a Lennox-Gastaut Syndrome (LGS), a Myoclonic Atonic Epilepsy (Doose Syndrome), a Rett Syndrome, a West Syndrome, or, Epilepsies Associated with Specific Genetic or Metabolic Disorders: a GLUT1 Deficiency Syndrome, a Neuronal Ceroid Lipofuscinosis (NCL), a Protocadherin 19 Female-Limited Epilepsy (PCDH19 Epilepsy), Pyridoxamine 5′-Phosphate Oxidase Deficiency, Trisomy 21 and Epileptic Spasms, TSC associated seizures or other rare or refractory epilepsies, optionally Epilepsy with Continuous Spike-Wave during Sleep (CSWS): Febrile Infection-Related Epilepsy Syndrome (FIRES) and medically intractable epilepsies, a complex partial seizures, infantile spasms, transitional focal epilepsy in infancy, a status epilepticus, partial seizures, catamenial epilepsies, early myoclonic encephalopathy, persistent spike epilepsy in slow wave sleep (except Landau-Kleffner syndrome), hypothalamic epilepsy, dose syndrome (myoclonic-standing disability epilepsy), myoclonic states of nonprogressive encephalopathy, tawnian syndrome or early epileptic encephalopathy in infancy, glycine encephalopathy, 15q repeat syndrome (Dup 15 q), epilepsy associated with CHD2, cyclin-dependent kinase-like 5 (CDKL 5), SCN1A, SCN2A, SCN8A, ARX, KCNA1, KCNA2, KCNT1, KCNQ2, HCN1, PCDH19, GRIN1, GRIN2A and GRIN2B mutations, a Smith-Magenis Syndrome, Ip36 deletion syndrome, 16pl I.2 deletion syndrome, fragile X syndrome, trisomy 21, Angelman's Syndrome, Schaff-Yang Syndrome, Albright Hereditary Osteodystroph, Silver-Russell Syndrome, Alstrbm Syndrome, Mental Retardation or WAGR or WAGRO Syndrome; Gillespie syndrome or cerebellar ataxia, Coffin-Lowry, hypersomnia, excessive daytime sleepiness (EDS), excessive daytime sleepiness associated with central or obstructive sleep apnea, idiopathic hypersomnia, sleep related breathing disorders, obstructive sleep apnea, central sleep apnea, Sleep-Related Hypoventilation Disorders, Sleep-Related Hypoxemia disorder, Rapid eye movement (REM) sleep behavior disorder, restless leg syndrome, nightmare disorder, dream anxiety disorder, Alpers Disease, Alpers Syndrome, Kearns-Sayre syndrome (KSS). Leber hereditary optic neuropathy (LHON), Leigh disease, Leigh syndrome, Mitochondrial encephalopathy lactic acidosis and stroke-like episodes (MELAS), Myoclonic epilepsy and ragged-red fiber disease syndrome (MERRF), Pontocerebellar Hypoplasia Type 6 (PCH6), Autism or Autism Spectrum Disorder (ASD), Asperger spectrum, or a Pervasive Developmental Disorder (PDD), hypersomnia, excessive daytime sleepiness (EDS), excessive daytime sleepiness associated with central or obstructive sleep apnea, idiopathic hypersomnia, narcolepsy, narcolepsy type 1, sleep related breathing disorders, obstructive sleep apnea, central sleep apnea, Sleep-Related Hypoventilation Disorders, Sleep-Related Hypoxemia Disorder, Rapid Eye Movement (REM) sleep behavior disorder, restless leg syndrome (RLS), nightmare disorder, dream anxiety disorder or a chronic fatigue syndrome, Parkinson's Disease and Alzheimer's Disease;
a neuropsychiatric disorder,
and optionally the neuropsychiatric disorder is T SC-Associated Neuropsychiatric Disorder (TAND), General Anxiety, Social Anxiety Disorder (Social Phobia), attention deficit hyperactivity disorder (ADHD) disruptive mood dysregulation disorder (DMDD), intermittent explosive disorder (IED), major depressive disorder (MDD), treatment resistant depression (TRD), obsessive-compulsive disorder (OCD), negative symptoms of schizophrenia, Parkinson's Disease, MDD Associated with Parkinson's Disease, general anxiety, Social Anxiety Disorder (Social Phobia), oppositional defiant disorder (ODD);
a retinopathy,
and optionally the retinopathy is a retinopathy of prematurity (ROP), Norrie disease retinopathy, Familial Exudative Vitreoretinopathy (FEVR), Coats' disease retinopathy or diabetic retinopathy;
a cardiomyopathy,
and optionally the cardiomyopathy is infantile hypertrophic cardiomyopathy, infantile dilated cardiomyopathy, infantile restricted cardiomyopathy, Barth's syndrome, mitochondrial cardiomyopathy; a renal disease, and optionally, the renal disease is nephrotic syndrome;
a peripheral nervous system disorder;
a peripheral nervous system disorder,
and optionally the peripheral nervous system disorder is Hereditary Neuropathy with liability to Pressure Palsies (HNPP), Neurofibromatosis Type 1 (NF1), Neurofibromatosis Type 2 (NF2), Schwannoma;
a neuroendocrine system disorder,
and optionally the neuroendocrine system disorder is Pheochromocytomas (PHEO), paragangliomas (PGL), pheochromocytomas and paragangliomas (PPGL), Hereditary paraganglioma-pheochromocytoma (PGL/PCC),
an allograph organ rejection prophylaxis, and optionally receiving allograft renal transplants, allograft liver transplants, allograft heart transplant;
a benign tumor, and wherein optionally the benign tumor is tuberous sclerosis complex (TSC)-associated subependymal giant cell astrocytoma (SEGA), cardiac rhabdomyoma, renal angiomyolipoma, PTEN hamartoma tumor syndrome (PHTS), Bannayan-Riley Ruvalcaba Syndrome (BRRS), Birt-Hogg-Dube Syndrome, Cowden's Syndrome, Proteus Syndrome or PTEN-related Proteus Syndrome;
a malignant neoplasm or cancer, wherein optionally the malignant neoplasm is a breast cancer, a hormone receptor positive breast tumor, a pancreatic neuroendocrine (pNET) or a renal cell carcinoma (RCC);
an autoimmune disorder, wherein optionally the autoimmune disorder is Crohn's Disease, Diabetes Type 1, Multiple Sclerosis, Rheumatoid Arthritis, Lupus, Scleroderma and/or Psoriasis,
a dermatological disorder, for example, psoriasis, urticaria or angioedema;
a pulmonary disease, wherein optionally the pulmonary disease is a lymphangioleiomyomatosis (LAM); and/or is pulmonary arterial hypertension (PAH); and/or
a polyposis, wherein optionally the polyposis is familial adenomatous polyposis (FAP) or hereditary flat adenoma syndrome or a Juvenile polyposis syndrome (JPS),
comprising administering to an individual in need thereof a therapeutic amount of:
a drug and companion diagnostic combination of claim 1.

11: The method of claim 10, wherein the one drug, or the two or more or all of the drugs in the drug and companion diagnostic, or therapeutic drug combination and companion diagnostic, are formulated in a unit dosage amount ranging from between 0.1 mg to about 1 gram, and optionally the one or two or more or all of the drugs are formulated as an immediate release formulation or a controlled release formulation.

12: The method of claim 10, wherein:
(a) the individual in need thereof is being treated for DMD and the method further comprises administration of a gene therapy comprising use of casimersen (or AMONDYS 45™);
the individual in need thereof is being treated for DMD and the method further comprises administration of a gene therapy comprising use of eteplirsen (or EXONDYS 51™),
the individual in need thereof is being treated for DMD and the method further comprises administration of a gene therapy comprising use of viltolarsen (or VILTEPSO™, or VYONSIA 53™),
the individual in need thereof is being treated for DMD and the method further comprises administration of a gene therapy comprising use of ataluren (or TRANSLARNA™),
the individual in need thereof is being treated for DMD and the method further comprises administration of a gene therapy comprising use of PF-06939926™ (Pfizer),
the individual in need thereof is being treated for DMD and the method further comprises administration of a gene therapy comprising use of SRP-9001™ (Sarepta Therapeutics, Roche),
the individual in need thereof is being treated for DMD and the method further comprises administration of a gene therapy comprising use of SPR-5051™ (Sarepta Therapeutics), or
the individual in need thereof is being treated for DMD and the method further comprises administration of a gene therapy comprising use of DS-5141b™ (Daiichi-Sankyo).

13: The method of claim 10, wherein the dosaging of:
(a) amifampridine is to attain a predicted blood or plasma Area Under the Curve (AUC)o-4h; target for amifampridine of between about 20 to 300 ng/mL per hour (ng/mL*h), between about 50 to 250 ng/mL*h, between about 100 to 200 ng/mL*h, or 150 ng/mL*h and to obtain a predicted blood or plasma AUC target precent coefficient of variation between about 5% to 35%, between about 5% to 25, between about 5% to 15% or between about 5% to 10%;
(b) deflazacort is to attain a blood or plasma AUCo-4h or AUCo-sh or AUCo-uhtarget for 21-desDFZ (the active metabolite of deflazacort) of between about 50 and 1000 ng/mL*h, about 100 to 600 ng/mL*h, between about 150 to 400 ng/mL*h, between about 200 to 300 ng/mL*h or 250 ng/mL*h8 and to obtain a predicted blood or plasma AUC target precent coefficient of variation between about 5% to 35%, between about 5% to 25, between about 5% to 15% or between about 5% to 10%;
(c) propranolol is to attain a predicted blood or plasma AUCo-9n target for propranolol of between about 100 to 1,000 ng/mL*h, between about 200 to 800 ng/mL*h, between about 350 to 600 ng/mL*h or 500 ng/mL*h; or C9h of between about 10 to 100 ng/mL, between about 20 to 80 ng/mL*h, between about 35 to 60 ng/mL*h or 50 ng/mL*h and to obtain a predicted blood or plasma AUC target precent coefficient of variation between 5% to 35%, between 5% to 25, between 5% to 15% or between 5% to 10%;
(d) serabelisib is to attain a predicted blood or plasma AUCo-24h target for serabelisib of between about 1000 to 25,000 ng/mL*h, between about 3,000 to 20,000 ng/mL*h, between about 5,000 to 15,000 ng/mL*h or 10,000 ng/mL*h and to obtain a predicted blood or plasma AUC target precent coefficient of variation between about 5% to 35%, between about 5% to 25, between about 5% to 15% or between about 5% to 10%;
(e) sirolimus is to attain a predicted blood C24h target for sirolimus of between about 1 to 20 ng/mL, between about 5 to 15 ng/ml, between about 10 to 15 ng/mL or 12 ng/mL and to obtain a predicted blood or plasma AUC target precent coefficient of variation between about 5% to 35%, between about 5% to 25, between about 5% to 15% or between about 5% to 10%; or,
(f) vamorolone is to attain a predicted blood or plasma AUCo-24h target for vamorolone of between about 400 to 2,000 ng/mL*h, between about 600 to 1,500 ng/mL*h, between about 800 to 1,200 ng/mL*h or 1,000 ng/mL*h and to obtain a predicted blood or plasma AUC target precent coefficient of variation between about 5% to 35%, between about 5% to 25, between about 5% to 15% or between about 5% to 10%;
(g) ataluren is to attain a predicted blood or plasma C6h target for ataluren of between about 1 to 20 μg/mL, between about 2 to 15 μg/mL, between about 5 to 10 μg/mL or 7 μg/mL and to obtain a predicted blood or plasma AUC target precent coefficient of variation between about 5% to 35%, between about 5% to 25, between about 5% to 15% or between about 5% to 10%, and/or
(h) everolimus to attain a predicted blood C24h target for everolimus of between about 0.5 ng/mL to 40 ng/mL, between about 1 to 20 ng/mL, between about 5 to 15 ng/ml, between about 10 to 15 ng/mL or 12 ng/mL and to obtain a predicted blood or plasma AUC target precent coefficient of variation between about 5% to 35%, between about 5% to 25, between about 5% to 15% or between about 5% to 10%.

14: A product of manufacture comprising or manufactured or fabricated as: a plurality of packages or packettes, or a blister packet, lidded blister or blister card or packets, or a shrink wrap, comprising or having contained or packaged therein a drug or a therapeutic combination of drugs, and a companion diagnostic, of claim 1.

15-16. (canceled)

17: The therapeutic drug and companion diagnostic combination of claim 1, wherein a therapeutic drug and a companion diagnostic combination packaged in a kit, a blister pack, a packet, a lidded blister, a blister card, a clamshell, a tray or a shrink wrap.

18: The therapeutic drug and companion diagnostic combination of claim 1, further comprising a time stamp for each drug container or receptacle, comprising or having contained therein at least one drug, drug combination or formulation.
